(19) United States
(12) Patent Application Publication
(10) Pub. No.: US 2025/0236648 A1
(43) Pub. Date: Jul. 24, 2025

(54) CONSTITUTIVE PRODUCTION OF MICROCINS TO TARGET ENTERIC BACTERIA

(71) Applicant: UNIVERSITY OF MASSACHUSETTS, Boston, MA (US)

(72) Inventors: Vanni BUCCI, Concord, MA (US); Benedikt M. MORTZFELD, Newton, MA (US)

(21) Appl. No.: 18/719,340

(22) PCT Filed: Dec. 16, 2022
(86) PCT No.: PCT/US22/53183

§ 371 Date: Jun. 13, 2024

Related U.S. Application Data
(60) Provisional application No. 63/290,649, filed on Dec. 16, 2021.

(51) Int. Cl. C07K 14/245; A61K 38/00; C12N 15/70

(52) U.S. Cl. CPC C07K 14/245; C12N 15/70; A61K 38/00; C12N 2800/101

(57) ABSTRACT

This disclosure relates to genetically engineered microorganisms for treating or reducing the risk of bacterial infections or dysbiosis, and further discloses methods of making and using such microorganisms.

### CLAIM OF PRIORITY

This application claims the benefit of U.S. Provisional Patent Application Ser. No. 63/290,649, filed Dec. 16, 2021. The entire contents of the foregoing applications are hereby incorporated by reference.

### FEDERALLY SPONSORED RESEARCH OR DEVELOPMENT

This invention was made with Government support under Grant No. W81XWH-20-2-0013 awarded by the Department of Defense. The Government has certain rights to the invention.

### TECHNICAL FIELD

This disclosure relates to genetically engineered microorganisms and methods of use.

### BACKGROUND

Medical complications related to drug-resistant bacteria (bacteria that cannot be effectively treated with currently available antibiotics), including those from members of the family Enterobacteriaceae, are a major issue in modern healthcare due to the increased morbidity, mortality, length of hospitalization and related healthcare costs. The CDC estimates that every year more than two million people acquire multi-drug-resistant bacterial infections, which result in over 23,000 directly-related deaths and more lethal outcomes from associated complications. A 2014 report from the World Health Organization (WHO) found that all WHO surveyed regions are characterized by high-rates of multi-drug resistant microorganisms, which are responsible for common health care facility and community acquired urinary tract infections (UTIs), pneumonias and blood stream infections. Of particular gravity is the fact that WHO's surveillance data are showing that more than 50% of Klebsiella species-related infections in all WHO regions are resistant to third generation cephalosporin, with a significant portion (>20%) also showing concurrent resistance to its only alternative, carbapenem.

Modern health care is challenged by the increasing emergence of multidrug resistant (MDR) pathogens, including carbapenem-resistant (CR) Enterobacteriaceae, which are responsible for millions of infections, tens of thousands of deaths, and billions of dollars in health care costs every year (Kadri S S. Key Takeaways From the U.S. CDC's 2019 Antibiotic Resistance Threats Report for Frontline Providers. Crit Care Med. 2020; 48(7):939-45; Cassini A et al. Attributable deaths and disability-adjusted life-years caused by infections with antibiotic-resistant bacteria in the EU and the European Economic Area in 2015: a population-level modelling analysis. The Lancet Infectious Diseases. 2019; 19(1):56-66; and Bartsch S M et al. Potential economic burden of carbapenem-resistant Enterobacteriaceae (CRE) in the United States. Clin Microbiol Infect. 2017; 23(1):48.e9-.e16). In 2017 the World Health Organization (WHO) released a list of bacteria, for which new antibiotics are urgently needed, and considered CR Enterobacteriaceae including Klebsiella pneumoniae, Escherichia coli, Salmonella enterica, or Enterobacter cloaceae of the highest priority (Tacconelli E et al. Discovery, research, and development of new antibiotics: the WHO priority list of antibiotic-resistant bacteria and tuberculosis. Lancet Infect Dis. 2018; 18(3):318-27). While K. pneumoniae is not known to cause gastrointestinal (GI) disease, its stable colonization of the intestine is the main reservoir for infections and transmission (Gorrie C L et al. Gastrointestinal Carriage Is a Major Reservoir of Klebsiella pneumoniae Infection in Intensive Care Patients. Clin Infect Dis. 2017; 65(2):208-15). MDR Klebsiella species are responsible for outbreaks in nursing homes and long-term care facilities (World Health O. Antimicrobial resistance: global report on surveillance. Geneva: World Health Organization; 2014) and for spreading antibiotic resistance among other members of the GI microbiota (Jacoby G A et al., Plasmid-Mediated Quinolone Resistance. Microbiology Spectrum. 2014; 2(5):2.5.33; and Wong M H Y, Chan E W C, Chen S. Evolution and Dissemination of OqxAB-Like Efflux Pumps, an Emerging Quinolone Resistance Determinant among Members of Enterobacteriaceae. Antimicrobial Agents and Chemotherapy. 2015; 59(6):3290-7). Moreover, GI colonization by certain K. pneumoniae contributes to the development of inflammatory bowel disease (Atarashi K et al. Ectopic colonization of oral bacteria in the intestine drives T(H)1 cell induction and inflammation. Science. 2017; 358(6361):359-65) and fatty acid liver disease (Yuan J et al. Fatty Liver Disease Caused by High-Alcohol-Producing Klebsiella pneumoniae. Cell Metab. 2019; 30(4):675-88.e7), making its selective removal a desireable milestone in modern medicine (Septimus E J, Schweizer M L. Decolonization in Prevention of Health Care-Associated Infections. Clin Microbiol Rev. 2016; 29(2):201-22). However, the paucity of treatment options for the elimination of CR K. pneumoniae begs for the development of new strategies to eradicate it from the GI tract.

A recent report has estimated that the economic burden for society due to Carbapenem-Resistant Enterobacteriaceae (CRE) infections, which include CR Klebsiella pneumoniae (CRKp), CR Klebsiella oxytoca (CRKo), CR Enterobacter cloaceae (CREc), and Enterobacter aerogenes (CREa), ranges from $37,000 to $83,000 per infection. When considering an infection incidence range of 2.93-15 per 100,000 people in the USA (i.e. 9,418-48,213 infections), CRE infections are estimated to cost society anything in the order of $1-$2 billion every year and to cause the loss of up to 45,261 quality-adjusted life years. Thus, there is pressing need to develop novel therapeutics that selectively kill pathogenic bacteria, reduce infection rates (and duration of infection), and curb the emergence of new drug-resistance mechanisms.

### SUMMARY

The present disclosure provides compositions of engineered microorganisms to produce microcins (e.g., microcin H47 or microcin I47) and methods of treating bacterial infections, e.g., gram-negative bacterial infections, and dysbiosis. The disclosure further provides genetically engineered microorganisms that include a microcin operon and a constitutive promoter for the microcin operon. In particular, the microcin operon includes one or more of mciI, mciA, mchA, mchC, mchD, mchE, and mchF genes. The constitutive promoter allows for continual transcription and expression of the one or more microcin genes. Either or both of the microcin operon and the constitutive promoter are heterologous to the microorganism. The microcin operon the constitutive promoter are in a self-retaining vector.

Accordingly, provided herein are genetically engineered microorganisms, wherein the microorganism comprises a microcin operon, and a promoter for the microcin operon, preferably wherein the promoter is a constitutive promoter, wherein the microcin operon comprises one or more microcin genes, and wherein either or both of the microcin operon and the constitutive promoter are heterologous to the microorganism; wherein the microcin operon and the promoter are in a vector, e.g., a self-retaining vector. In some embodiments, the constitutive promoter allows for the continual transcription and expression of the microcin operon. In some embodiments, the constitutive promoter drives the expression of the microcin operon constitutively.

In some embodiments, the genetically engineered microorganism is a bacterium.

In some embodiments, the genetically engineered microorganism is Escherichia coli.

In some embodiments, the E. coli is E. coli Nissle 1917 (EcN).

In some embodiments, the self-retaining vector comprises one or more genetic modifications of a native vector of the microorganism.

In some embodiments, the microorganism comprises a deletion of the native vector and the introduced self-retaining, genetically-modified vector.

In some embodiments, the self-retaining vector is a multicopy vector.

In some embodiments, the genetically engineered microorganism retains the self-retaining vector without antibiotic selection.

In some embodiments, the self-retaining vector is a pMUT1 or pMUT2 vector.

In some embodiments, the microcin operon comprises one or more Microcin H47 (MccH47) genes.

In some embodiments, the microcin operon comprises one or more of mchB, mchC, mchD, mchE, mchF, mchX mchI, and mchA.

In some embodiments, the microorganism comprises mchA.

In some embodiments, the microcins operon comprises one or more Microcin I47 (MccI47) genes.

In some embodiments, the microcin operon comprises microcin genes mciI, mciA, mchC, mchD, mchE, mchF and mchA.

In some embodiments, the microorganism comprises mchA.

In some embodiments, the microorganism comprises a deletion of the native Microcin H47 or Microcin M genes.

In some embodiments, the constitutive promoter is a J23119 promoter.

Also provided herein are self-retaining multicopy plasmid vectors comprising: a microcin operon comprising a set of microcin genes, and a constitutive promoter, wherein the constitutive promoter allows for continual transcription and expression of at least one microcin gene.

In some embodiments, the set of microcin genes comprises one or more Microcin H47 (MccH47) genes.

In some embodiments, the set of microcin genes comprises one or more of mchA, mchB, mchC, mchD, mchE, mchF, mchX, mchI and mchA.

In some embodiments, the microcins operon comprises one or more Microcin I47 (MccI47) genes.

In some embodiments, the microcin operon comprises microcin genes mciI, mciA, mchC, mchD, mchE, mchF and mchA.

In some embodiments, the constitutive promoter is a J23119 promoter.

In some embodiments, the vector is a plasmid.

In some embodiments, the vector is a pMUT1 or pMUT2 plasmid.

In some embodiments, the self-retaining multicopy plasmid vector comprises: the microcin operon comprising mciI, mciA, mchC, mchD, mchE, mchF; a first constitutive promoter, wherein the first constitutive promoter allows for continual transcription and expression of the operon; mchA, and a second constitutive promoter, wherein the second constitutive promoter allows for continual transcription and expression of the mchA.

In some embodiments, the self-retaining multicopy plasmid vector comprises: the microcin operon comprising mchB, mchC, mchD, mchE, mchF, mchX and mchI; a first constitutive promoter, wherein the first constitutive promoter allows for continual transcription and expression of the operon; mchA, and a second constitutive promoter, wherein the second constitutive promoter allows for continual transcription and expression of the mchA.

In some embodiments, the constitutive promoter is a J23119 promoter.

In some embodiments, the vector is a plasmid.

In some embodiments, the composition comprises any one of the genetically engineered microorganisms described herein.

In some embodiments, the composition is packaged in a capsule for intestinal delivery.

In some embodiments, the bacterial infection is a gram-negative bacterial infection.

In some embodiments, the bacterial infection is carbapenem-resistant Enterobacteriaceae infection, E. coli infection, Salmonella infection, and/or Shigella infection.

In some embodiments, the carbapenem-resistant Enterobacteriaceae infection is a Klebsiella pneumoniae infection.

Also provided herein are methods of treating intestinal dysbiosis, the method comprising: identifying a subject as having intestinal dysbiosis; and administering to the subject a therapeutically effective amount of a composition comprising any one of the genetically engineered microorganism described herein or any one of the compositions described herein.

In some embodiments, the subject is a human and the composition is administered by endoscopy, enteroscopy, colonoscopy, a nasoduodenal catheter, enema, or by oral administration.

In some embodiments, the composition is orally administered, optionally in a capsule.

Also provided herein are methods of treating a bacterial infection, the method comprising: identifying a subject as having a bacterial infection; and administering to the subject a therapeutically effective amount of a composition comprising any one of the genetically engineered microorganism described herein or any one of the compositions described herein.

In some embodiments, the subject is a human and the composition is administered by endoscopy, enteroscopy, colonoscopy, a nasoduodenal catheter, enema, or by oral administration.

In some embodiments, the composition is orally administered, optionally in a capsule.

In some embodiments, the bacterial infection is a gram-negative bacterial infection.

In some embodiments, the bacterial infection is carbapenem-resistant Enterobacteriaceae infection, E. coli infection, Salmonella infection, and/or Shigella infection.

In some embodiments, the carbapenem-resistant Enterobacteriaceae infection is a Klebsiella pneumoniae infection.

Also provided herein are methods of reducing a risk of a bacterial infection, the method comprising: identifying a subject as having a risk of a bacterial infection; and administering to the subject a composition comprising any one of the genetically engineered microorganism described herein or any one of the compositions described herein.

In some embodiments, the subject is being administered one or more antibiotics.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated herein by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a plasmid map of pBBAD-I47, a pUC19-based plasmid producing MccI47-MGE from mciA, the immunity peptide mciI as well as the genes needed for post-translational modification mchCDEFA.

FIG. 1B shows the results of static inhibition assays comparing MccH47 and MccI47 activity from single colony production against multidrug-resistant Escherichia coli (top) and Klebsiella pneumoniae (bottom). The control strain (ctrl) harbors the same backbone plasmid (pUC19) without microcin production.

FIG. 1C shows the results of static inhibition assays comparing purified MccH47 and MccI47 activity against multidrug-resistant Escherichia coli (top) and Klebsiella pneumoniae (bottom). After purification, 3.5 μg of each microcin were dried on the media before the overlay with the target bacteria. Scale bars: 1 cm.

FIG. 2A shows the curing of the native EcN plasmid pMut2 via utilizing inducible MccI47 expression as a counterselection tool. Counterselection plasmid pCure2-I47 with pMut2 as backbone was introduced into EcN by electroporation and chloramphenicol (Cm) supplementation. High concentrations of Cm led to complete displacement of pMut2 in the cells. Without further antibiotic selection, mciA expression was induced, leading to selection against pCure2-I47, since the immunity peptide mciI was omitted in the plasmid design.

FIG. 2B is a plasmid map of pMut2-I47, an MccI47-producing plasmid with pMut2 as backbone vector.

FIG. 2C shows the results of a serial passage experiment to assess pMut2-I47 plasmid retention in EcN^{ΔH ΔM}-I47. Cells were initially grown with the supplementation of ampicillin and then subjected to daily subculturing for ten days without selection pressure, resulting in a total of approximately 200 bacterial generations (n=5).

FIG. 2D shows the results of the assessment of plasmid copy number of pMut2 and the pMut2-based pMut2-I47 using quantitative PCR. Note that EcN^{ΔH ΔM}-I47 cells harboring pMut2-I47 exhibit a significantly higher copy number mean compared to EcN harboring native pMut2. Additionally, both plasmids are stably retained for the ten days of the serial passage experiment. ***: p<0.001 (n=5).

FIG. 2E shows the results of static inhibition assay comparing inhibitory activity of (1) EcN, (2) EcN^{ΔH ΔM}, and (3) EcN^{ΔH ΔM}-I47, harboring pMut2-I47, against carbapenem-resistant Klebsiella pneumoniae. Inhibitory activity of EcN^{ΔH ΔM}-I47 is independent ampicillin (Amp) for plasmid selection. Scale bar: 1 cm.

FIG. 3A is a schematic representation of the experimental design. Mice were treated with ampicillin in the drinking water for seven days and infected with 10⁸ cells of carbapenem-resistant K. pneumoniae (BAA 1705). After one week of engraftment, the mice were treated daily with either PBS, or with 10⁸ cells of the different EcN strains (EcN, EcN^{ΔH ΔM} and EcN^{ΔH ΔM}-I47) for seven days by oral administration.

FIG. 3B shows the Log₂ fold change of K. pneumoniae colonization throughout the treatment with PBS or the respective EcN strain (EcN, EcN^{ΔH ΔM} and EcN^{ΔH ΔM}-I47). Linear mixed effect modeling was used to assess the change in K. pneumoniae (BAA 1705) colonization. *: p<0.05, ***: p<0.001. n=16.

FIG. 3C is a principal coordinate analysis (PCoA) of the bacterial communities before and after treatment. The experimental time introduces significant differences between the samples pre and post treatment. Ellipses are grouping samples according to time point pre and post treatment (n=8).

FIG. 4 is a plasmid map of pBBAD-H47, a pUC19-based plasmid producing MccH47-MGE from mchB, the immunity peptide mchI under an L-arabinose inducible PBAD promoter as well as the genes needed for post-translational modification mchCDEFA. AmpR=ampicillin resistance, X=mchX, I=mchI, B=mchB.

FIG. 5A is a plasmid map of pCure2-I47. Grey shaded area indicates the pMut2 backbone. CmR=chloramphenicol resistance.

FIG. 5B shows the results of gel electrophoresis of PCR products for EcN strains created during the process pMut2 curing and transformation with pMut2-I47. (1) Primers against pMut1 (1208 bp), (2) primers against the insert-free pMut2 backbone (1296 bp), (3) primers against pCure2-I47 (1493 bp), (4) primers against pMut2-I47 (244 bp). Asterisks indicate specific fragments amplified by PCR. 1 kb Plus DNA Ladder (New England Biolabs, Ipswich, MA).

FIG. 6 shows the genetic organization of the E. coli Nissle 1917 class IIb microcin gene cluster. Wildtype EcN (top) harbors genes for production of the microcins MccH47 (mchB) and microcin MccM (mcmA) including their corresponding immunity genes mchII and mcmI. To allow selective colony counting, a kanamycin resistance cassette was introduced into the EcN genome and the resulting strain served as the EcN control in this study (center, EcN). A MccH47/MccM knockout strain (bottom, EcN^{ΔH ΔM}) was generated to exclude the interference of native EcN microcin production, when assessing the potency of MccI47 against K. pneumoniae in vitro and in vivo. This strain served as the knockout control and the basis for the strain harboring the MccI47-producing plasmid pMut2-I47 (EcN^{ΔH ΔM}-I47). Shaded areas indicate sequence similarity between the depicted gene clusters. X=mchX, I=mchI, B=mchB, truncated mciI, *=flippase recognition target (FRT) site, KanR=kanamycin resistance.

FIG. 7 shows the growth curves of different EcN strains. Note that none of the genetic modifications had an impact on the growth rate. Wildtype EcN represents the native strain, while all other strains were created for this study (see Supplementary FIG. 6). EcN and EcN^{ΔH ΔM} serve as the study controls for EcN^{ΔH ΔM}-I47, which is expressing MccI47 from pMut2-I47 (n=8).

FIG. 8 shows Klebsiella pneumoniae (BAA 1705) colony forming units (CFUs) throughout the in vivo experiment. Linear mixed effect modeling. *: p<0.05, ***: p<0.001 (n=16).

FIG. 9A shows that differential species are significantly affected by the respective treatment compared to the baseline EcN^{ΔH ΔM}-I47 (linear mixed effect modeling; Speciesᵢ(counts)˜Time*Treatment+I\MouseID; p≤0.05).

FIG. 9B are boxplots of affected species before and after treatment. SN=significant negative change, SP=significant positive change, CPM=counts per million.

### DETAILED DESCRIPTION

Delivery of rationally-designed combinations of gastrointestinal commensal bacteria has the benefit of ensuring CRE decolonization via a number of concurring mechanisms including competition for nutrient and space, production of antimicrobial molecules and immune-system stimulation. However, the cost of large-scale production of these consortia linearly scales with the number of employed species (1-2 months per strain based on work from our industrial partners), thus making the generation of consortia of dozens of strains a big and time-consuming endeavor. Recent work has shown that addition of single strains of microcinogenic intestinal residents (i.e. bacteria capable of secreting small antimicrobial peptides) can lead to the killing of pathogenic Gram-negative Enterobacteriaceae, and therefore could be used as novel live biotherapeutics. However, because native microcin production is performed by strains with unknown mammalian gut colonization capability, and is dependent on the conditions experienced in the intestine (e.g., iron limitation), this phenomenon is difficult to control and thus exploit for therapies.

Under the global societal challenge of moving towards more personalized medicine, biomedical research is currently exploring the use of engineered bacteria as vehicles for the targeted administration of therapeutic compounds at the location of disease and with the goal of reducing systemic side effects (Charbonneau M R et al. Developing a new class of engineered live bacterial therapeutics to treat human diseases. Nat Commun. 2020; 11(1):1738; and Adolfsen K J et al. Improvement of a synthetic live bacterial therapeutic for phenylketonuria with biosensor-enabled enzyme engineering. Nature Communications. 2021; 12(1):6215). This is particularly relevant when the therapeutic objective is the delivery of protein-based agents (i.e., AMPs) to the GI tract as host digestive enzymes likely inactivate them before they reach their site of action (Starr C G et al., Antimicrobial peptides are degraded by the cytosolic proteases of human erythrocytes. Biochim Biophys Acta Biomembr. 2017; 1859(12):2319-26). Furthermore, embedding the proteins into a lipid matrix within enteric capsules to prevent host-mediated degradation (Maroni A et al., Enteric coatings for colonic drug delivery: state of the art. Expert Opinion on Drug Delivery. 2017; 14(9):1027-9), needs to account for the agent's dilution effect that naturally occurs downstream of the point of capsule's opening. These issues are circumvented with the use of live, engineered bacteria as they can be designed to ensure continuous production of the therapeutic compound while passing through the GI tract (Ozdemir T et al., Synthetic Biology and Engineered Live Biotherapeutics: Toward Increasing System Complexity. Cell Systems. 2018; 7(1):5-16).

Current antibiotics are often applied systemically to treat bacterial pathogens, yet increasingly fail to clear infections caused by MDR organisms (Petros A J et al. Systemic antibiotics fail to clear multidrug-resistant Klebsiella from a pediatric ICU. Chest. 2001; 119(3):862-6). Additionally, these drugs also cause profound gastrointestinal dysbiosis of the protective microbiota, leaving treated individuals with often dangerous secondary outcomes (Willing B P et al., Shifting the balance: antibiotic effects on host-microbiota mutualism. Nature Reviews Microbiology. 2011; 9(4):233-43). Selective decolonization of pathogenic bacteria as a way to minimize their translocation and horizontal transmission to other patients is currently being explored in the pharmaceutical industry by assembling consortia of isolated bacteria that display decolonizing properties against certain species, including K. pneumoniae (Feehan A et al. Bacterial, Gut Microbiome-Modifying Therapies to Defend against Multidrug Resistant Organisms. Microorganisms. 2020; 8(2); and Dsouza M et al. Colonization of the live biotherapeutic product VE303 and modulation of the microbiota and metabolites in healthy volunteers. Cell Host Microbe. 2022; 30(4):583-98.e8). However, to our knowledge, there is no live biotherapeutic for pathogen decolonization that has reached the stage of human trials.

Described herein are rationally engineered biotherapeutic E. coli Nissle 1917 that are capable of microcin overproduction. Compared to previous studies that either rely on genomic integration (which would lead to a single copy of the operon and thus suboptimal production) or rely on the use of plasmids that require a strong selective pressure for their maintenance (Praveschotinunt P et al., Engineered E. coli Nissle 1917 for the delivery of matrix-tethered therapeutic domains to the gut. Nat Commun. 2019; 10(1):5580), the present strains use a constitutive expression system from a stably-retained multicopy plasmid that allows for high MccI47 output, while rendering provisions for plasmid selection unnecessary. The data herein show clinical relevance of the engineered MccI47-producing strains in an in vivo model of CR K. pneumoniae colonization. Specifically, MccI47 production provided a significant decline in intestinal CR K. pneumoniae abundance compared to a mock treatment or a control strain without microcin production after seven days of daily oral probiotic administration, with no major effect on the resident microbiota. Selective reduction in gastrointestinal CR K. pneumoniae colonization, including hypervirulent strains, in symptomatic and asymptomatic carriers may decrease host-to-host transmission and thus ameliorate host pathogenesis in affected populations.

Described herein is the use of engineered probiotics and engineered live biotherapeutic products for the selective removal of MDR pathogens from the GI tract.

Members of drug-resistant Enterobacteriaceae spp. include opportunistic pathogens (e.g., Salmonella spp.) that are among the leading causes of morbidity and mortality worldwide. Overgrowth of these bacteria is considered a hallmark of intestinal dysbiosis. Some gut commensals produce microcins, small antimicrobial peptides, that inhibit growth of select pathogens. As described herein, select gut commensals can be genetically altered and used to effectively treat pathogenic bacteria infections and/or to limit the growth of pathogenic bacteria.

Described herein are E. coli probiotics that constitutively produce microcins, including H47 and I47. Performing in vitro experiments using both heterologous I47 production from a probiotic or I47, purified for the first time by us, we observed that microcin I47 is especially effective in killing CR K. pneumoniae, suggesting that we have identified a novel molecule for the killing of this deadly pathogen.

The disclosure also provides plasmid-based systems capable of producing microcins such as microcin H47 or microcin I47. The disclosure also provides the use of mature (post-translationally modified) microcin delivered from a probiotic or in its purified form as an antibiotic to kill bacteria, e.g., Klebsiella species with a specific focus on drug-resistant Klebsiella species. As shown herein, I47 can also kill other Enterobacteriaceae including Escherichia coli, Salmonella Typhimurium, Salmonella Typhi, and Shigella flexneri. Additionally, this disclosure provides genetically engineered probiotics capable of conditionally producing microcins such as microcin H47 or microcin I47.

### Microcins

Microcins are low-molecular-weight antimicrobial peptides secreted by members of the Enterobacteriaceae family. They include, e.g., Class I microcins, Class IIa microcins, Class IIb microcins, and Class IIc microcins. Class I microcins have molecular masses <5 kDa, are post-translationally modified, and bind to a spectrum of targets. Class IIb microcins are relatively large (˜5-10 kDa) polypeptides and feature a C-terminal siderophore post-translational modification. Class IIb microcins include, e.g., Microcin H47 (MccH47, also referred to as H47 herein), MccE492, MccM, MccG492 and MccI47 (also referred to as I47 herein). See, e.g., PCT Publication Nos. WO 2019/055781, WO 2020/227155, and WO 2021/173545.

### Mcch47

Microcin H47 (MccH47 or H47) is a bactericidal antibiotic. Due to its size, it shares with other microcins the ability to pass through cellophane membranes. MccH47 has been shown to be active to inhibit various bacteria, e.g., gram-negative bacteria, E. coli, Salmonella, Enterobacter, Shigella, Klebsiella, and Proteus spp. The genes required for production of MccH47 are clustered in a 10-kb DNA segment located in the E. coli chromosome and include the genes: mchA, mchB, mchC, mchD, mchE, mchF, mchI, mchX, mchS1, and mchS4. Four genes, mchA, mchB, mchC, and mchD, are devoted to MccH47 synthesis; an immunity gene, mchI, encoding a small, 69-residue integral membrane peptide; and two further genes, mchE and mchF, are required for the secretion of the antibiotic into the extracellular medium.

A small gene, mchX, was found upstream of the immunity determinant; preliminary results point to its involvement in the activation of its own expression and probably in that of downstream immunity and production genes. The mchX, mchI, and mchB genes are located in the central region of the MccH47 genetic system, and are often referred as mchXIB. They are known to be transcribed in the same direction, towards mchB. Notably, the mchX gene may be involved in the activation of its own expression and the activation of downstream immunity and production genes.

MccH47 production is a process involving three main steps: synthesis of the precursor peptide MchB, subsequent maturation of the molecule, and its final secretion. These MccH47 genes are described, e.g., in Vassiliadis et al. (2010) Isolation and characterization of two members of the siderophore-microcin family, microcins M and H47, Antimicrobial agents and chemotherapy 54.1:288-297, which is incorporated herein by reference in its entirety. MccH47 production is a process involving three main steps: synthesis of the precursor peptide MchB, subsequent maturation and post-translational modification of the molecule, and secretion of the molecule. The complexity of the MccH47 antibiotic system parallels that of other microcin systems, such as those of microcins B17 and C7. MccH47 maturation, in which mchA, mchC, and mchD gene products are known to be necessary, is believed to endow the antibiotic molecule with the ability to enter cells.

An example amino acid sequence of MccH47 is shown in SEQ ID NO: 16 below:

>CAD56182.1 MchB protein [Escherichia coli]
(SEQ ID NO: 16)
MREITESQLRYISGAGGAPATSANAAGAAAIVGALAGIPGGPLGVVVGAV
VSAGLTTAIGSTVGSGSASSSAGGGS

### Mcci47

Microcin I47 is a bactericidal antibiotic. Due to its size, it shares with other microcins the ability to pass through cellophane membranes. Microcin I47 has been reported to be produced by the MccH47 genetic system and detected in iron deprivation conditions (Azpiroz et al., 2011, PLOS ONE 6(10):e26179; Poey et al., 2006, Antimicrob Agents Chemother 50(4):1411-8).

Production and purification of microcin I47 can be conducted by any suitable method known in the art. In some embodiments, microcin I47 can be purified using an amylose resin column eluted with maltose. For example, cultures of E. coli producing microcin I47, e.g., E. coli NEB10β pHMT-I47, are grown under antibiotic selection (e.g., ampicillin and/or chloramphenicol), and in iron-limiting conditions, e.g., via the addition of 0.2 mM 2′2-dipyridyl, and induced, e.g., with isopropyl β-d-1-thiogalactopyranoside (IPTG). Cultures are grown for an additional time, e.g., 4 to 10 hours, e.g., 5 to 7 hours, post-induction, then pelleted and frozen overnight, e.g., at −20° C.

Cultures can then be thawed in cold water, sonicated, and the crude lysate is passed through a resin column, e.g., an amylose resin (New England Biolabs, Ipswich, MA) column, to capture maltose-binding protein (MBP) fusion proteins, then finally eluted, e.g., with maltose. Elution is performed by adding the elution buffer (e.g., 200 mM NaCl, 20 mM Tris-HCl, 10 mM maltose; pH 7.5).

The eluent can be concentrated, for example, using MilliporeSigma (Burlington, MA) MWCO 10,000 filters. The concentrated MBP-MccI47 is then digested by an endopeptidase, such as the Tobacco etch virus nuclear-inclusion-a endopeptidase (TEV) (New England Biolabs, Ipswich, MA), yielding a buffered solution of MccI47, TEV, and MBP. This solution can then be further purified, e.g., by subsequent rounds of resuspension with Ni-NTA agarose resin (Qiagen, Hilden, DE). Ni-NTA slurry can be pelleted by centrifugation and the supernatant can be removed by pipetting.

An example amino acid sequence of I47 is shown in SEQ ID NO: 17 below:

(SEQ ID NO: 17)
MREISDNMLDSVKGGMNLNGLPASTNVIDLRGKDMGTYIDANGACWAPDT
PSIIMYPGGSGPSYSMSSSTSSANSGS

### Genetic System for Producing MccI47 and MccH47

The genes required for production of MccI47 and MccH47 are clustered in a 10-kb DNA segment located in the E. coli chromosome and include the genes: mchA, mchB, mchC, mchD, mchE, mchF, mchI, mchX, mciA (formerly known as mchS2), mciI (formerly known as mchS3), mchS1, and mchS4. Three genes, mchA, mchC, and mchD, are devoted to mature microcin synthesis; whereas mciA and mciI encode for the precursor and the corresponding immunity peptide of MccI47 and mchB and mchI for the precursor and the corresponding immunity peptide of MccH47. Two further genes, mchE and mchF, are required for the secretion of the antibiotic into the extracellular medium.

Production of class IIb microcins is a process involving three main steps: synthesis of the precursor peptide, subsequent maturation of the molecule, and its final secretion. These microcin genes are described, e.g., in Vassiliadis et al. (2010) Isolation and characterization of two members of the siderophore-microcin family, microcins M and H47, Antimicrobial agents and chemotherapy 54.1:288-297, which is incorporated herein by reference in its entirety. The complexity of the MccI47 antibiotic system parallels that of other microcin systems, such as those of microcins B17 and C7. MccI47 maturation, in which mchA, mchC, and mchD gene products are known to be necessary, is believed to endow the antibiotic molecule with the ability to enter cells.

mchA gene sequence
(SEQ ID NO: 1)
ATGCGAAAACGTATTCTTTTTATTGGCCCACCGCTGTACGGTTTGTTATA
CCCATTGATTTCTCTGGCTCAGGCCTTTCGTGTAATCGGACATGATGTAG
TAATTAGTAGTGCTGGCAAATTCGCGAATAAAGCAGCAGAAGCTGGACTG
GTTGTTTTTGATGCAGTTCCAGGTTTAGATTCAGAGGCTGGATATCGCCA
TCAGGAAGAGTTGAGGAAAAAAAGTAATATTATTGGTCATTTCTCTTTTT
TTAGCGATGAAATGGCAGATAACCTCATCGATTTTGCAGGAAAATGGAGG
CCAGATTTAATAGTCTATCCCCCGCTTGGTCCGGCAGGCCCATTGGTTGC
TGCTAAATATAGAATTCCTTCAGTGATGCTGGCTGTTGGATTCGCGCATA
CATCTGCCCATATTCAGATGTTAAACCGTTCTTTAAGCAATGCTTACAGG
CGGCATGGAGTCAGCGGTCCACTATGTGATTTAGCATGGATTGATGTTGC
TCCCCCAAGTATGAGCATTCTTAAAAATGCTGAAGAACCGGTTATCTCAA
TGAGATATATTCCTTATAACGGAGGTGCTGTAAAGGAAACATGGTGGGAC
AGGGATTCTGATCGAAAACGTTTACTCATCAGCCTTGGCACTGTAAAACC
AATGGTTGATGGTCTGGAGCTGATTTCATGGGTTATGGATTCTGCAAATG
AAGTTGATGCTGATATCATTTTGCAACTTGCAATAAATGCTCGTACTGGA
TTACGAAAACTACCATCAAATGTACGTCTGGTTGACTGGATACCTATGGG
TGTATTCCTTAATGGAGCTGATGGATTTATTCATCATGGTGGCGCAGGTA
ATACCCTGACAGCGTTGTATAGTGGGATACCACAGATTGTGTTTGGCGAA
GGTGCAGATCGCTCTGTTAATGCAGAAATTGTTGCGATGCGTGGGTGTGG
GATTATTCCGGACAAGCATGGACTGACCAGTGATTTGGTAAATCGCCTGC
TTTATGATGATTCACTACGCTTCTGTTCAGATCAGGTAGCCGCTGAAATG
GCTGAACAACCCAGTCCTGCAGAGATCGCAGAGGTTTTGATGAGAAAATT
AAAAAACAACGGGAAATAA.
mchC gene sequence:
(SEQ ID NO: 2)
ATGAGTCATCAGTGTTCACTTTCTGAACTGAATGAAAACCTGGTGCCTTT
CACTGCCAGGCAGATCAAGTCCTCATTAATCTGGTGTGCAGAGGATGTCA
GAAATCCAGGCGAGCTGCAAAATGCCTGCAGTTATATTATCGATCCTGAC
AGTACGGCTTCTGCCAAAGTGTTCCATGCAGAGCGCTATGGTGGCAGTGG
TATTCAGCGTAATGGAGGTGGTGCACGTTGTGGGTTTGATGGTAACTACC
AGGTTAAAGGAATAGGAAGTAATCCGTTGGTTGGTGAAGGTACTGACGAA
CGTCATTCTAATGGTGCACTCGGCGCTGTTCATGCAATATATGAGGCTTT
GTGGGGAGAAGTACTGGCTCAAATATTACCTTATAGTGCTGTGCGGGTTC
GGGCGGTTTTACTTACAGATCTCTATACTGAAAAGGCATTTGAGCGCTCC
GGTATGAAATCACGAAGAGCCCTGTTGGTACGTGAGCCTGTTGTTCGCCC
GGCGCATTTTGAACGGGCACCATACTTCCAAGTAAAACCGGAGTATTCCA
GTCAGTTAATTCACGATGCCTGTCGGGTTAGATCTGTGATCCACAAGCTG
CCAGGATATCTACCTGTACCACCGGAAGAAATTGATGCTGAAGCACGAAC
TGATCCCCGGATTTATTGCATTGAGGGATTATGTGAACTGGCACGTCGTG
AGGCCTGGCAAATGGCATTTTGTCGAACACGTTTCCTGAGATTGACAACT
TCTCCTTCTAATATTGCAATGGATGGCAGATTAATGGATTTTAACGGACT
CAGTTGCTCGTTTCCGGGAGATTCCCCAGCTGATTTTGGGTATAAACTAA
GATTAGCTGAACTGGCAAAAGAACCGATGGTACTTATGCAAGGGCTGTCT
GATCTCTGCTTGTATATCGGAAAATATATGTTTGACCCTGACTTCACTCT
TGCAGCCCGTTTGAAGGTTGAGGAGATATTTCAGAAAACTTTTCATGAAG
CATGTTATTACTGTTATCTAGAACTGTTGGGTATTCCTGGAGAATTTATA
ACACAAAAAGAGATACCTGATATATTGAAACAACTGGTTAACAGTTTTGT
TGCATTACTCAATAAATACTGCGAGAAATCACATGCCCAAGATATTGTCA
ATCAGGATGGTTCACCATTGCAAAAGTTGGTTGTGACGCTAATCCATCAT
AGGCATAATCAAAAGCAGGCACTGAATAGTAGCATCAAGAATGATGTTTA
TTTCACCGTTGCACAACAGTGTTTTTCCCAGACTATCCACTGGCTGACGC
AAGGCAGTACCAGACGTCAGATAAATGCTTCATTACTCCTGAAAGAAATT
GAACATCATACCATGAAAAGGCTGCAACCCAGGGAAGAGCTGAGGAAAGA
GAATATGTGCGAAAAAATTGCCATCCTGCTGGATAATCATGGCGATGATC
CCCTTTTTTTACAAGAAGCAATTTCTGATATGAAAAATTTTATGCTTAAG
TTTTCCAGAGATGCATTTGGATATCTTGAACCGATAAGAAACACAGTGTA
A.
mchD gene sequence:
(SEQ ID NO: 3)
ATGTCTTATATAAGGGAAACCATCAGAGGAAAAGATGAATGGACTGTTTA
TGAACAGATAGGTTTTGCGGTCAGTTGTATGCTCTACAATCGTAATTACA
GTCTGTATCCGGTGTTAACCATTCAATACTGGACTGAATATGCGATACAG
CATAATCAGATTAAATTCCTGTTTGATTCACGAGGTTTTCCACTGGCGTA
TATAACCTGGGCATATCTTGAGGCTGATACGGAAGCGCGCCTGCTCAGGG
ATCCAGAATTCAGGTTGCATCCGTCTGAATGGAATGAAGATGGAAGGATC
TGGATCCTGGATTTCTGTTGTAAACCAGGCTTTGGTCGAAAAGTTATTGA
CTATCTCATACAGCTTCAGCCATGGGGGGAAGGAGAAGTACGATGGTTAA
GCAGGCGAAAGAAAATTGTGACATACATCCCTGAGCGGCTGCATAAAACG
TAG.

The mchB genes encodes the pre-microcin H47 peptide. Once the peptide product of the mchB gene has gone through modification and secretion steps, the pre-microcin H47 peptide becomes microcin H47.

mchB gene sequence:
(SEQ ID NO: 4)
ATGCGAGAAATAACAGAATCACAGTTAAGATATATTTCCGGGGGGGGAGG
TGCGCCAGCGACTTCAGCTAATGCCGCAGGTGCTGCAGCTATTGTTGGAG
CTCTCGCCGGAATACCTGGTGGTCCACTTGGGGTTGTAGTTGGAGCCGTA
TCTGCCGGTTTGACAACAGCAATTGGCTCGACCGTGGGAAGTGGTAGTGC
CAGTTCTTCTGCTGGTGGCGGTAGCTAA.

The mchE and mchF genes encode secretion proteins, which are necessary for secretion out of the cell. In some embodiments, the secretion proteins encoded by mchE and mchF are required for export of the microcin, but are not required for the production of the microcin.

mchE gene sequence:
(SEQ ID NO: 5)
TTGTTTCGTCAGGATGCTTTAGAAAACAGAAAAATGAAGTGGCAGGGACG
GGCAATATTACTTCCCGGAATACCACTATGGTTAATCATGCTGGGAAGCA
TTGTGTTTATTACGGCATTTCTGATGTTCATTATTGTTGGTACCTATAGC
CGCCGTGTTAATGTCAGTGGTGAGGTCACAACCTGGCCAAGAGCTGTCAA
TATATATTCAGGTGTACAGGGATTTGTTGTCAGGCAATTTGTTCATGAAG
GGCAGTTGATAAAAAAAGGGGATCCTGTTTATCTGATTGACATCAGTAAA
AGTACACGTAGTGGTATTGTCACTGATAATCATCGGCGGGATATAGAAAA
TCAGCTGGTTCGTGTGGACAACATTATTTCCCGTCTGGAAGAAAGTAAAA
AAATAACGTTAGATACCCTGGAAAAACAACGTCTGCAATACACAGATGCG
TTTCGTCGCTCATCAGATATTATACAGCGTGCAGAGGAAGGGATAAAAAT
AATGAAAAACAATATGGAGAATTACAGAAACTATCAGGCAAAAGGGCTGA
TTAATAAAGATCAGTTAACTAACCAGGTGGCATTATATTATCAGCAACAA
AACAATCTTCTCAGCCTGAGCGGACAGAACGAACAGAATGCCCTGCAGAT
AACCACTCTGGAGAGTCAGATTCAGACTCAGGCTGCAGATTTTGATAACC
GTATCTACCAGATGGAACTGCAACGGTACGAGTTACAGAAAGAACTGGTT
AACACTGATGTGGAGGGCGAAATTATTATCCGGGCGTTGACTGACGGGAA
AGTTGACTCCCTGAGTGTCACTGTCGGGCAAATGGTCAATACCGGAGACA
GCCTTCTGCAGGTTATTCCTGAGAACATTGAAAACTATTATCTTATTCTC
TGGGTCCCAAATGATGCTGTTCCTTATATTTCGGCTGGTGACAAAGTGAA
TATTCGTTATGAAGCCTTTCCGGCAGAAAAATTTGGGCAGTTCTCTGCTA
CGGTTAAAACTATATCCAGGACTCCTGCGTCAACACAGGAAATGTTGACC
TATAAGGGTGCACCACAGAATACGCCGGGCGCCTCTGTTCCCTGGTATAA
AGTCATTGCGATGCCTGAAAAGCAGATTATCAGATATGACGAAAAATACC
TCCCTCTGGAAAATGGAATGAAAGCCGAAAGTACACTATTTCTGGAAAAA
AGGCGTATTTACCAGTGGATGCTTTCTCCTTTCTATGACATGAAACACAG
TGCAACAGGACCGCTCAATGACTAA.
mchF gene sequence:
(SEQ ID NO: 6)
ATGACTAACGGGAGTTTCAGACAAATTATAAATCAGCTTGATATGCGCTG
GCGACGTCGTGTTCCGGTTATTCATCAGACGGAGACCGCTGAATGTGGAC
TGGCCTGCCTGGCAATGATATGCGGTCATTTTGGTAAGAATATTGACCTG
ATATCTCTTCGCCGGAAGTTTAATCTCTCGGCCCGTGGAGCAAACCTTGC
AGGAATCAATGGAATAGCGGAGCAGCTGGGGATGGTCACCCGGGCTCTTT
CACTGGAGCTGGATGAACTTGGTGCCCTCAAAATGCCGTGTATTCTCCAC
TGGGATTTCAGTCACTTTGTCGTGCTGGTCAGCGTAAAGCGTAACCGTTA
TGTACTGCATGATCCGGCCAGAGGCAGAAGATATCTCGGTCGGGAGGAAA
TGAGCCGGTATTTTACGGGCATTGCACTTGAGGTCTGGCCTGGAAGTGAA
TTCCTGGCGGAAACCCAGCAGATCCGCATAAGTCTCCGTTCACTGATTAA
CAGTATTTACGGTATTAAAAGAACACTGGCGAAAATTTTCTGTCTGTCAG
TTGTAATTGAAGCAATCAATCTGGTAATGCCGGTGGGGACTCAGCTGGTT
ATGGATCATGCGATTCCGGCGGGGGACAGAGGGCTGCTGACGCTTATTTC
TGCTGGCCTGATGTTCTTTATATTGCTCAGGGCCGCGGTGAGTATGCTGC
GTGCATGGTCCTCACTGGTTATGAGCACGCTCATCAATATACAGTGGCAG
TCGGGTCTGTTTAACCATCTTCTCAGACTGCCGCTGGCGTTTTTTGAACG
CCGTAAATTAGGTGATATCCAGTCGCGTTTTGGCTCCCTTGACACTTTGA
GGGCCACCTTTACCACCTGTGTGGTTGGGGCAATCATGGACAGTATTATG
GTTGTGGGGGTTTTTGTGATGATGCTGTTATATGGAGGATATCTTACCTG
GATAGTGCTCGGTTTTACCATGGTTTACGTTCTTATTCGTCTGGTGACAT
ACGGCTATTACCGGCAAATATCGGAAGAAACTCTTGTCAGGGGGGCCCGG
GCCAGCTCCTATTTTATGGAAAGCCTGTATGGTATTGCCACGGTAAAAAT
CCAAGGTATGGCTGGGATCCGGGGAACACACTGGCTTAACCTGAAAATAG
ATGCGATCAATTCAGGTATTAAGTTAACCAAGATGGATTTGCTCTTCGGG
GGGATAAATACTTTTGTTGCCGCCTGTGATCAGGTGGCGATTTTATGGCT
GGGTGCAAGCCTTGTGATCGATAATCAGATGACAATAGGGATGTTTGTGG
CATTTGGTTCTTTTCGTGGGCAGTTTTCGGATCGGGTTGCTTCGCTGACC
AGTTTTCTTCTTCAACTGAGAATAATGAGTCTGCATAATGAGCGCATTGC
AGATATTGCACTACATGAAAAGGAAGAAAAGAAACCGGAAATTGAAATCG
TTGCTGACATGAGCCCGGTTTCACTGGAAACCACTGATTTAAGCTACCGG
TATGACAGCCAGTCAGCACAGGTATTCAGTGGTCTGAATTTGTCTGTGGC
TCCGGGAGAAAGTGTGGCTATAACTGGTGCCTCCGGTGCCGGAAAAACCA
CATTAATGAAAGTATTATGTGGACTGTTTGAACCAGATAGTGGAAAAGTA
CTGGTTAATGGCACGGATATACGTCAACTTGGAATAAATAATTATCACCG
TATGATAGCCTGTGTTATGCAGGACGACCGGCTATTTTCAGGATCAATTC
GTGAAAATATCTGTGGGTTTGCAGAAGAAACAGACGACGAATGGATGACA
GAATGTGCCAGAGCAAGTCATATTCATGATGTGATAATGAAAATGCCAAT
GGGGTATGAAACGTTAATAGGTGAACTGGGGGAAGGTCTTTCCGGCGGTC
AAAAACAGCGTATATTCATTGCCCGAGCTTTATACCGGAAACCTGGAATA
TTATTTATGGATGAGGCTACAAGTTCTCTTGATACAGAAAGTGAACGTTT
CGTGAATGCTGCCATAAAAAAAATGAATATCACCCGGGTGATTATTGCAC
ACAGAGAAACTACGTTGAGAACTGTTGACAGGATTATTTCTATTTAA.

The mchI gene encodes for the MccH47 immunity protein.

mchI gene sequence:
(SEQ ID NO: 7)
ATGAGTTATAAAAAACTGTACCAATTGACGGCTATATTTAGTTTACCTCT
TACTATCTTATTGGTTTCACTTTCATCCCTTCGGATTGTTGGCGAAGGGA
ATTCTTATGTTGACGTTTTTCTAAGCTTTATAATATTTCTTGGTTTTATT
GAGCTGATTCATGGGATTCGAAAGATTTTGGTCTGGTCAGGCTGGAAAAA
CGGAAGTTAA.
mchX gene sequence:
(SEQ ID NO: 8)
ATGGAATTTGCTACAAACAGGGTTACTGTAAATGACAGTCGGTCAGCACT
GTCATCAACTTTGCTGTTGTCTTTGATCATGAGCGCCACTCTACTGGAAT
ATTCTTTATCGATGACCTGA.
mchSI gene sequence:
(SEQ ID NO: 9)
ATGAAAAACTATCTTTTCCAGACTCCCGAAGATATTTGTGTACAGTTAAA
AAAAATGACACATCCTGTCACAATAAGAACAACAGATATTGCTAATTTCT
GGCACTATCTTGAGTCAGCAACTCTTCCGGTGATCACAAAAAGCACCACT
ACAGAAAATCGGGAGGTTACATTTCTGTGGCGCTCAGAGAAAGCAGTGCA
AGGCGTATATCTTCGCCTGAATCGTGTTACAGATAAAAAAGATGTCAAAA
AAGGACTAATGACTCATATCCCTTCGACAGATATCTGGATGCTGACACTG
GTGTTACCAGCTTCATATCGGGGCTCATACTCATTTATAGAAATTCCCAC
AGATATGACACAAAAAGACATATTTCAACTAGGAAGTCGCTTCTCTCCAT
TACCCGGTAAATCTGATCCATTTAACAAAACAGCAGAAATAAATATACGA
GGATTCGGAGAATCAGTCCTTTCTCTTGATATGGCTCCTGAACAAAAGGA
ATGGGATGATACTTCCCATAAATGTACAGGTATTCTTTCAACATTACATT
CCTTTGTTGCAGGATATCAACGCCGGATTCGTTTATATTTTCCCCAGAAT
CCAACATCAGTACCTCTTGGATTACTTGTGTTACCTGATGCTGAAATATG
GTTTGACCGGATGGATATTACCCGGGCATTAGATATGGCCATTACCACTG
GTCATATTGCGCCAATGGCAATTATGGGGATAGACAATATTAATGAATCT
GATCGTATGAATATACTGGGAGGCAATAAAGAACTTATCTTTGATATAGC
GGAAAATCTGATACCCCAGTTATACAGAGACTACCCGAATATCGTATGGG
CTGGTCGTTCTAATACTATACTGGCCGGTCAGAGCCTCGGTGGAGTGACA
GCACTGATGGCAGCTATATATGCGTCGACAACATTTGGTACAATCATTAG
CCACTCACCTTCAATGTGGTGGAACCCTGACCAGGGCAGCCCGATTTTGT
TTACTGAGAATGATATCTCCTGGGTAAGTGAGCAGATACTTTCAGCGCCT
CCGAAAGATGTAAATATCCAACTTGGAGTCGGTTCTTTAGAAGGTACAAC
CGTCTCACATGTTCAGCGGTTGCATCAGTCGTTAATCGCAGCAGGTTTGG
AAAGTAACCTCACTGTCTATGCCGGTGGTCATGATTATGCCTGGTGGCGC
GGAGCAATTATTGATGCATTAGCAAATTATAATTGCAGGAAGATATCAGA
TAATAACTTTGTGTAA.
mchS4 gene sequence:
(SEQ ID NO: 10)
ATGAATTGTGATAATAATCACAGAAATGAAGAATTCATTGTTACCTTTGA
TAAAGGCAACAAGCAAGACAATTCAAGACGAAAACACGATAATTTTCCTA
TAGAGGTAGAATCCTCCGTAGAGCTGGAGACACACTGTATCACAAATAAT
AAGTCGGCTTCCGGTATAGTAACACATGACTATGATGCCGATTATATTTG
TGGTTGTGGTGAAATTATGTGTCCTGGTTGCGGTCATGACCTATAA.

The mciA (formerly known as mchS2) gene encodes the pre-microcin I47 peptide. Once the peptide product of the mciA gene has gone through modification and secretion steps, the pre-microcin I47 peptide becomes microcin I47.

(SEQ ID NO: 11)
ATGAGAGAAATATCAGATAACATGCTTGATTCCGTGAAAGGAGGGATGAA
TCTTAATGGATTACCTGCTTCTACTAATGTAATAGATCTACGTGGAAAAG
ATATGGGAACATATATTGATGCTAATGGAGCATGCTGGGCTCCGGATACT
CCATCCATCATCATGTATCCGGGGGGAAGTGGACCTTCTTATAGTATGAG
TAGTTCCACATCCAGTGCAAACAGCGGCAGTTAA

The mciI (formerly known as mchS3) gene encodes for the MccI47 immunity protein.

mciI gene sequence:
(SEQ ID NO: 12)
ATGTATCTTACGAAAAAGATTATAATAAGTATGATGTTTATATTACCATC
TGCTGCATTTTCATCAGATCCACCTCCCCTTCAACAATCGTTAGAAAAAA
CAACCTATTTTTCTATAGGTATGAATGGGTTTATAGGCTATCAGAGCGAA
GGGGAAAAATTATACACACACATTCTTACATTAGATAATCCCGAAGAGAT
ATTTAAAAATATAATAAAAAATAGAAAGTCAACTAAGGAGTCTAAAATTT
ATGCTGCTTGTGGGCTATATTATTTAAACGTAGAAAATATAGAGTCATTG
TTTAATGAAAATGATAAACAAGAATATGTGTCTGTCTTAAGAGGGGATAT
TTTAACAAAAATAAAACTGAATGATATTCTGAATTCTGTGATAATAAATG
GTTGCAACACCAAATTAATATCTGAACATAAATGA.

### Vectors

This disclosure provides various vectors comprising microcin genes and constitutive promoters. In one aspect, provided herein are self-retaining multicopy plasmid vectors comprising: a microcin operon comprising a set of microcin genes, and a constitutive promoter, wherein the constitutive promoter allows for continual transcription and expression of at least one microcin gene.

In some embodiments, the constitutive promoter is a J23119 promoter. In some embodiments, the constitutive promoter is aproD promoter.

The vector can include genes for various microcins, e.g., Class I microcins, Class IIa microcins, Class IIb microcins, and/or Class IIc microcins. In some embodiments, the vector can include a set of genes for a Class IIa microcin (e.g., MccH47, MccE492, MccM, MccG492, and MccI47).

In some embodiments, the vector includes a set of genes for MccH47. These genes are required to express a functional MccH47 that can inhibit the growth of other bacteria. In some embodiments, the vector can include a set of genes for MccH47. In some embodiments, the set of genes for MccI47 includes one, two, three, four, five, six, seven, or eight genes that are selected from the group of mchA, mchB, mchC, mchD, mchE, mchF, mchX and mchI.

In some embodiments, the vector for the production of active, mature microcin H47 is constructed by combining a plasmid expressing mchX mchI and mchB, a plasmid expressing mchC, mchD, mchE, and mchF, and a plasmid expressing mchA. In some embodiments, the vector for the production of active, mature microcin H47 is constructed by combining a plasmid expressing mchX mchI and mchB, a plasmid expressing mchC, mchD, mchE, and mchF, and a plasmid expressing mchA, and mchS4. In some embodiments, the vector for the production of active, mature microcin H47 is constructed by combining a plasmid expressing mchX mchI and mchB, a plasmid expressing mchC, mchD, mchE, and mchF, and a plasmid expressing mchA, mchS1 and mchS4. In some embodiments, the plasmids are combined into a single backbone vector, e.g., pMUT2.

In some embodiments, the self-retaining multicopy plasmid vector comprises: the microcin operon comprising mchB, mchC, mchD, mchE, mchF, mchX and mchI; a first constitutive promoter, wherein the first constitutive promoter allows for continual transcription and expression of the operon; mchA, and a second constitutive promoter, wherein the second constitutive promoter allows for continual transcription and expression of the mchA.

In some embodiments, the vector includes a set of genes for MccI47. These genes are required to express a functional MccI47 that can inhibit the growth of other bacteria. In some embodiments, the set of genes includes one, two, three, four, five, six, or seven genes that are selected from the group consisting of mchA, mchC, mchD, mchE, mchF, mchX, mchI, mchS1, mchS4, mciI and mciA. In some embodiments, the set of genes for MccI47 includes one or more of mciI, mciA, mchC, mchD, mchE, mchF and mchA controlled under a J23119 promoter.

In some embodiments, the vector for the production of active, mature microcin I47 is constructed by combining a plasmid expressing mciI and mciA, a plasmid expressing mchC, mchD, mchE, and mchF, and a plasmid expressing mchA. In some embodiments, the vector for the production of active, mature microcin I47 is constructed by combining a plasmid expressing mciI and mciA, a plasmid expressing mchC, mchD, mchE, and mchF, and a plasmid expressing mchA, and mchS4. In some embodiments, the vector for the production of active, mature microcin I47 is constructed by combining a plasmid expressing mciI and mciA, a plasmid expressing mchC, mchD, mchE, and mchF, and a plasmid expressing mchA, mchS1 and mchS4. In some embodiments, the plasmids are combined into a single backbone vector, e.g., pMUT2.

In some embodiments, the self-retaining multicopy plasmid vector comprises: the microcin operon comprising mciI, mciA, mchC, mchD, mchE, mchF; a first constitutive promoter, wherein the first constitutive promoter allows for continual transcription and expression of the operon; mchA, and a second constitutive promoter, wherein the second constitutive promoter allows for continual transcription and expression of the mchA.

In some embodiments, one or more genes in a set of genes are located within one operon. In some embodiments, the set of genes are located within more than one operons. Thus, in some embodiments, the operon includes one, two, three, four, five, six, seven, eight, or nine, ten, eleven or twelve genes.

In some embodiments, the set of genes or the operon is under the control of a constitutive promoter. As used herein, the term “constitutive promoter” refers to a an unregulated promoter that allows for continual transcription and expression of its associated gene.

Any suitable constitutive promoter can be used in the methods and vectors described herein. Examples of constitutive promoters are 114018, 114033, 114034, 1732021, 1742126, J01006, J23100, J23101, J23102, J23103, J23104, J23105, J23106, J23107, J23108, J23109, J23110, J23111, J23112, J23113, J23114, J23115, J23116, J23117, J23118, J23119, J23150, J23151, J44002, J48104, J54200, J56015, J64951, K088007, K119000, K119001, K1330002, K137029, K137030, K137031, K137032, K137085, K137086, K137087, K137088, K137089, K137090, K137091, K1585100, K1585101, K1585102, K1585103, K1585104, K1585105, K1585106, K1585110, K1585113, K1585115, K1585116, K1585117, K1585118, K1585119, K1824896, K2486171, K256002, K256018, K256020, K256033, K292000, K292001, K418000, K418002, K418003, K823004, K823005, K823006, K823007, K823008, K823010, K823011, K823013, K823014, M13101, M13102, M13103, M13104, M13105, M13106, M13108, M13110, M31519, R1074, R1075, and S03331. Further characteristics and sequences of the example promoters are available at the igem.org website.

In some embodiments, the constitutive promoter is J23119. In some embodiments, the constitutive promoter is proD. In some embodiments, the constitutive promoter is a ProD-like insulated J23119 promoter, which is an insulated ProD promoter with the promoter region being replaced by J23119.

In some embodiments, the constitutive promoter (J23119) is located immediately upstream of the microcin genes. In some embodiments, the mchA can controlled by a constitutive promoter (e.g., J23119).

J23119 promoter sequence:
(SEQ ID NO: 13)
TTGACAGCTAGCTCAGTCCTAGGTATAATGCTAG.
A ProD-like insulated J23119 promoter sequence is
shown in SEQ ID NO: 14:
(SEQ ID NO: 14)
ACAGCTAACACCACGTCGTCCCTATCTGCTGCCCTAGGTCTATGAGTGGT
TGCTGGATAACTTGACAGCTAGCTCAGTCCTAGGTATAATGCTAGCAGGG
AGACCACAACGGTTTCCCTCTACAAATAATTTTGTTTAACTTTAGAGGAG
GTAATA

In some embodiments, the set of genes or the operon is under the control of a controllable promoter. In some embodiments, mechanisms can be introduced to the genetically engineered microorganisms to control the transcription of the genes or the operon, and thus control the level of microcins. The transcription of the microcin genes can be controlled by a controllable promoter (i.e., inducible promoter). In some embodiments, the set of genes or the operon is under the control of a controllable promoter. Some exemplary controllable promoters include, but are not limited to, Pttr promoter or pBAD promoter. The pBAD promoter is found in bacteria and was originally part of the arabinose operon that regulates transcription of araB, araA, and araD. Transcription initiation at the pBAD promoter occurs in the presence of high arabinose and low glucose concentrations. Upon arabinose binding to AraC, the N-terminal arm of AraC is released from its DNA binding domain via a “light switch” mechanism. This allows AraC to dimerize and bind the I1 and I2 operators. The AraC-arabinose dimer at this site contributes to activation of the pBAD promoter. Additionally, cyclic AMP receptor protein (CAP) binds to two CAP binding sites upstream of the I1 and I2 operators and helps activate the pBAD promoter. In the presence of both high arabinose and high glucose concentrations however, low cAMP levels prevent CAP from activating the pBAD promoter. In the absence of arabinose, AraC dimerizes while bound to the O2 and I1 operator sites, looping the DNA. The looping prevents binding of CAP and RNA polymerase. Thus, without arabinose, the pBAD promoters are repressed by AraC. A detailed description of pBAD promoter can be found, e.g., in Schleif R. AraC protein, regulation of the L-arabinose operon in Escherichia coli, and the light switch mechanism of AraC action. FEMS Microbiol. Rev., (2010) 1-18, which is incorporated by reference in its entirety.

This disclosure further provides genetically engineered microorganisms comprising the vectors as described herein. Thus, in one aspect, this disclosure provides an engineered strain of EcN harboring a plasmid-based system carrying the operon including one or more microcin genes described herein, capable of producing a microcin (MccH47 or MccI47) in response to environmental tetrathionate, resulting in the ability to inhibit and out-compete an infectious bacteria.

### Genetically Engineered Microorganisms

Many microorganisms can be genetically engineered to treat bacterial infection as described herein. In some embodiments, a bacterium is used. In some embodiments, the bacterium is E. coli (e.g., E. coli Nissle 1917 or E. coli NGF-19). One useful E. coli strain is Nissle 1917 (EcN), a Gram-negative species that is easily cultured, easily genetically manipulated, able to colonize a human host, and easy to use for human probiotic applications. EcN is the active component of Mutaflor® (Ardeypharm GmbH, Herdecke, Germany), a microbial probiotic drug that is marketed and used in several countries. Clinical trials have shown EcN to be effective for maintaining remission of ulcerative colitis (UC), for stimulation of the of the immune system in premature infants, for treatment of infectious GI diseases, for the relief of constipation, and also for treatment of Irritable Bowel Syndrome in some patients.

In some embodiments, useful microorganisms that can be used in the methods disclosed herein include bacteria also used for making yogurt, e.g., Lactobacillus delbrueckii subsp. bulgaricus and Streptococcus thermophiles.

A vector or a set of genes as described herein can be introduced into a microorganism, e.g., a bacterium, such as, E. coli, to generate a genetically engineered microorganism by known molecular biology, microbiology, and recombinant DNA techniques. These techniques are familiar to one of skilled in the art and are explained fully in the literature. See, e.g., Molecular Cloning: A Laboratory Manual (Michael R. Green, Joseph Sambrook, Fourth Edition, 2012); Oligonucleotide Synthesis: Methods and Applications (Methods in Molecular Biology) (Piet Herdewijn, 2004); Nucleic Acid Hybridization (M. L. M. Andersen, 1999); Short Protocols in Molecular Biology (Ausubel et al., 1990), each of which is incorporated herein by reference in its entirety.

In some embodiments, the microcin operon and the constitutive promoter are in a self-retaining vector. As used herein, a bacterium containing a “self-retaining” vector is used to refer to a bacterial host cell carrying non-native genetic material propagated on a self-replicating extra-chromosomal vector (e.g., plasmid), such that the non-native genetic material is retained, expressed, and propagated. The bacterium is capable of survival and/or growth in vitro, e.g., in medium, and/or in vivo, e.g., in the gut. For example, the stable bacterium may be a genetically modified bacterium comprising a genetically-modified pMUT2 self-retaining plasmid, in which the plasmid carrying the microcin operon is stably maintained in the host cell, such that microcin can be expressed in the host cell, and the host cell is capable of survival and/or growth in vitro and/or in vivo.

In some embodiments, the vector or the set of genes is not integrated into the bacterial or other microbial genome.

In some embodiments, the self-retaining vector is a multicopy vector. As used herein, a “multicopy vector” refers to a small and highly prevalent vector (e.g., plasmid) in bacteria, that typically range from 10 to 30 copies per cell. In some embodiments, the genetically-modified microorganism described herein contains about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more copies of the self-retaining plasmid.

In some embodiments, the self-retaining vector is a cryptic vector present in the bacterium. For example, EcN natively contains plasmids pMUT1 and pMUT2, which have no known function but are retained within the bacteria (Kan et al., ACS Synth. Biol. 2021, 10, 1, 94-106). In some embodiments, the self-retaining plasmid is a pMUT2 plasmid. An example nucleic acid sequence of a pMUT2 plasmid carrying the MccI47 genes is shown in SEQ ID NO: 15

>pMut2-147
(SEQ ID NO: 15)
TTTTGATCTCTTTGGTGCGCTTTTCCATGAGAATCCGCTGAGAAAGTTTCGCACCCAAAGTGCGAATTTTCGCAGTGGATGCAAGG
GGTTTCGGGGGGCGGCGAGCCCCCTGAAACAGTCACAGACGGCACCTCGAAGAGGGGACGCTGTGTGTACTGGCTTAGTACAGCAT
CTATCGTACATCGAGGTCGCATCACGCACAAACAAAAAGCCCCGCAAAAGCAGGGCTGTTATCTGATATAGGTTGTTTTGTCTCAC
ACGGCAGCGGAAGAGGAATCCGAAGTGGTACTGGTAGTAGTATTGGATGCTGCTGACGATATTTTCCGCTTTGACCCAAGGCTTAA
ATAATCAATGCCTGTAATCAACGATCTCAATACGCCTTCGGATACCATAGCGATAAACGTATCTTGCTGGTTATGGCTTGCGATGC
AAATCGTAGCATCACCTTTTTTATACTTTAAAACACCTGCTAAATATCCATTTTCATCTAGAACACTCTTAAGATGTTCATTTGTT
ATTGTTTGTAGCGTTTGCTTTGTTTCGCTTCGAGCATACGCCTTAGCTAGCTTCCGAGAAAAAGCATCCGCATCATGACTATCTTT
ATTTACTCGCTCAATAAATTTGCTTAAGTCAACAAATCCCTTAAAACGAGTGGACATATTGTTAACAAAATCAGTGGCAGCATTTT
TTATCCATGCTTTATAGCCAAAAAAACGCTCGAAAACATTTTGGTCGTAGATAAATACCGTATCGCCAGCAAAAACAAGAGATGCC
TTACCATCAATAGAAATCATATCTTGATCTACTCGAGATAGTTCTTTTTTGCTAAACCCATAAAAATTATTTTTCTTTCTTGATAA
GTTTTGAACTGGATATTGCTTCTTGTATATGGTTATACAATTGTCGTCATTATTCTTACTCAAAACGAAAATGATTGAGTCAACTT
TTGATATTAGATCCACACTGTCAAAATCAACAATTGGGATATTTTCATTGCCATCACCACCAAACCCAGCATCAAACACAATGCCA
GTCAGAAACTTCATTTGCTCATTATCATACTGCTCAGCATCATTTAAAAATGAAATGGTGTTGGTGCGGTCACTTAGTGTATTAAC
ATCTGATACTATCACAACCCGATTTTCTAAATCAGATATAGTTTTCTTTATCACATTATCAATAATGGACTGTTTTAGCTCACTGT
CATTTTTAAGGATGGCAATTTTATAGCTAAAAGAGTCCTTAGCACCCGCTTTACCTTTATTTTTAAAGTTAAAGTAAGTGTGCAAT
GTAACATCGTTAATATCACAATCAAAATGCTTATACAGTTCTAAAAGCTCTTGTGCTTTTTCTTCATTATGCTCCAAAGCATCAAG
ATCTAAGGCATCGTCACTCATCATCATTCCTCTATGATTTTTTTCGCGAACGTTAAATAATCATCATGATTTATAACTCTGATAAA
ATCATTTTCTTTTATTAAATCTTTAGATAAAACTATCAACTCACCGTCTTGCGTTTTTTCCCTTCCATTAGCTACCACACTGTAAG
TAATCTTATAGGCAGAAACATTAAATAATGACAATGTTGGGTTGCAGTGAATTCTTTTTGTTTTGATGTGCAAAAAACCGACGATA
ATCAAAACAAACAAAAAATTAACTATATTTGATGGTTTGCTTAAATCAGTAAAGACCAACGGCATTATGTACGTTGATAAAAAAGA
AAGATACTCACCGGATTCTTTTTTACATGAAACGACCTTTAACTTTCTTGACACCGCACCGGAGTCTAAGTTTTTCAAAACCCATC
GATACCAAATGTATGTATAAGAACAAGTTAAAATCAAAGCCCCGCAGATCACTGACCTCAATACAGAAAATGTTAATCTGCTATTT
GAATAGTCGAGTACGCATTGAAATTTTCCATCCGCGCCAGGTCATGAGATTATCAAAAAGGATCTTCACCAAGATCCTTTTAAATT
AAAAATGAAGTTTTAAATCAATCTAAAGTATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAACTACGATACGGGAGGGCTTACCATCTGG
CCCCAGTGCTGCAATGATACCGCGAGACCCACGCTCACCGGCTCCAGATTTATCAGCAATAAACCAGCCAGCCGGAAGGGCCGAGC
GCAGAAGTGGTCCTGCAACTTTATCCGCCTCCATCCAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCGTCGTTTGGTATGGCTTCATTCAGCTCCGGTTCCCA
ACGATCAAGGCGAGTTACATGATCCCCCATGTTGTGCAAAAAAGCGGTTAGCTCCTTCGGTCCTCCGATCGTTGTCAGAAGTAAGT
TGGCCGCAGTGTTATCACTCATGGTTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATCTTACCGCTGTTGAGAT
CCAGTTCGATGTAACCCACTCGTGCACCCAACTGATCTTCAGCATCTTTTACTTTCACCAGCGTTTCTGGGTGAGCAAAAACAGGA
AGGCAAAATGCCGCAAAAAAGGGAATAAGGGCGACACGGAAATGTTGAATACTCATACTCTTCCTTTTTCAATATTATTGAAGCAT
TTATCAGGGTTATTGTCTCATGAGCGGATACATATTTGAATGTATTTAGAAAAATAAACAAATAGGGGTTCCGCGCACATTTCCCC
GAAAAGTGCCACAGCTAACACCACGTCGTCCCTATCTGCTGCCCTAGGTCTATGAGTGGTTGCTGGATAACTTGACAGCTAGCTCA
GTCCTAGGTATAATGCTAGCAGGGAGACCACAACGGTTTCCCTCTACAAATAATTTTGTTTAACTTTAGAGGAGGTAATAATGTAT
CTTACGAAAAAGATTATAATAAGTATGATGTTTATATTACCATCTGCTGCATTTTCATCAGATCCACCTCCCCTTCAACAATCGTT
AGAAAAAACAACCTATTTTTCTATAGGTATGAATGGGTTTATAGGCTATCAGAGCGAAGGGGAAAAATTATACACACACATTCTTA
CATTAGATAATCCCGAAGAGATATTTAAAAATATAATAAAAAATAGAAAGTCAACTAAGGAGTCTAAAATTTATGCTGCTTGTGGG
CTATATTATTTAAACGTAGAAAATATAGAGTCATTGTTTAATGAAAATGATAAACAAGAATATGTGTCTGTCTTAAGAGGGGATAT
TTTAACAAAAATAAAACTGAATGATATTCTGAATTCTGTGATAATAAATGGTTGCAACACCAAATTAATATCTGAACATAAATGAG
CAGGAGGTTTTTAAAATGAGAGAAATATCAGATAACATGCTTGATTCCGTGAAAGGAGGGATGAATCTTAATGGATTACCTGCTTC
TACTAATGTAATAGATCTACGTGGAAAAGATATGGGAACATATATTGATGCTAATGGAGCATGCTGGGCTCCGGATACTCCATCCA
TCATCATGTATCCGGGGGGAAGTGGACCTTCTTATAGTATGAGTAGTTCCACATCCAGTGCAAACAGCGGCAGTTAAATAAATACC
ATCCGGCTATGCCGGCAGGCACTTTTTCGATAGTGCCTGTAAAATATATCCCTTTCGTTTAAAGCAGATAGAAGATCCATTTATAA
GTGTGTTTTTATGTTCAACACTTTACTGATGAAGATATGATTTACAGTCGTTGATAAATAATTATTAATTTATGTGTAATTAATAG
AGTTTTACTGTAATAAGGAAGAAGCTGTCAGGACACTTACTTGCTCTTTTATGAACACTCTTTTATGAAAATTAAAACGGAGAATA
CTGCCAGATGAGTCATCAGTGTTCACTTTCTGAACTGAATGAAAACCTGGTGCCTTTCACTGCCAGGCAGATCAAGTCCTCATTAA
TCTGGTGTGCAGAGGATGTCAGAAATCCAGGCGAGCTGCAAAATGCCTGCAGTTATATTATCGATCCTGACAGTACGGCTTCTGCC
AAAGTGTTCCATGCAGAGCGCTATGGTGGCAGTGGTATTCAGCGTAATGGAGGTGGTGCACGTTGTGGGTTTGATGGTAACTACCA
GGTTAAAGGAATAGGAAGTAATCCGTTGGTTGGTGAAGGTACTGACGAACGTCATTCTAATGGTGCACTCGGCGCTGTTCATGCAA
TATATGAGGCTTTGTGGGGAGAAGTACTGGCTCAAATATTACCTTATAGTGCTGTGCGGGTTCGGGCGGTTTTACTTACAGATCTC
TATACTGAAAAGGCATTTGAGCGCTCCGGTATGAAATCACGAAGAGCCCTGTTGGTACGTGAGCCTGTTGTTCGCCCGGCGCATTT
TGAACGGGCACCATACTTCCAAGTAAAACCGGAGTATTCCAGTCAGTTAATTCACGATGCCTGTCGGGTTAGATCTGTGATCCACA
AGCTGCCAGGATATCTACCTGTACCACCGGAAGAAATTGATGCTGAAGCACGAACTGATCCCCGGATTTATTGCATTGAGGGATTA
TGTGAACTGGCACGTCGTGAGGCCTGGCAAATGGCATTTTGTCGAACACGTTTCCTGAGATTGACAACTTCTCCTTCTAATATTGC
AATGGATGGCAGATTAATGGATTTTAACGGACTCAGTTGTCTGTTTCCGGGAGATTCCCCAGCTGATTTTGGGTATAAACTAAGAT
TAGCTGAACTGGCAAAAGAACCGATGGTACTTATGCAAGGGCTGTCTGATCTCTGCTTGTATATCGGAAAATATATGTTTGACCCT
GACTTCACTCTTGCAGCCCGTTTGAAGGTTGAGGAGATATTTCAGAAAACTTTTCATGAAGCATGTTATTACTGTTATCTAGAACT
GTTGGGTATTCCTGGAGAATTTATAACACAAAAAGAGATACCTGATATATTGAAACAACTGGTTAACAGTTTTGTTGCATTACTCA
ATAAATACTGCGAGAAATCACATGCCCAAGATATTGTCAATCAGGATGGTTCACCATTGCAAAAGTTGGTTGTGACGCTAATCCAT
CATAGGCATAATCAAAAGCAGGCACTGAATAGTAGCATCAAGAATGATGTTTATTTCACCGTTGCACAACAGTGTTTTTCCCAGAC
TATCCACTGGCTGACGCAAGGCAGTACCAGACGTCAGATAAATGCTTCATTACTCCTGAAAGAAATTGAACATCATACCATGAAAA
GGCTGCAACCCAGGGAAGAGCTGAGGAAAGAGAATATGTGCGAAAAAATTGCCATCCTGCTGGATAATCATGGCGATGATCCCCTT
TTTTTACAAGAAGCAATTTCTGATATGAAAAATTTTATGCTTAAGTTTTCCAGAGATGCATTTGGATATCTTGAACCGATAAGAAA
CACAGTGTAATAAATATATAAGGGAAATAGTAATCATGTCTTATATAAGGGAAACCATCAGAGGAAAAGATGAATGGACTGTTTAT
GAACAGATAGGTTTTGCGGTCAGTTGTATGCTCTACAATCGTAATTACAGTCTGTATCCGGTGTTAACCATTCAATACTGGACTGA
ATATGCGATACAGCATAATCAGATTAAATTCCTGTTTGATTCACGAGGTTTTCCACTGGCGTATATAACCTGGGCATATCTTGAGG
CTGATACGGAAGCGCGCCTGCTCAGGGATCCAGAATTCAGGTTGCATCCGTCTGAATGGAATGAAGATGGAAGGATCTGGATCCTG
GATTTCTGTTGTAAACCAGGCTTTGGTCGAAAAGTTATTGACTATCTCATACAGCTTCAGCCATGGGGGGAAGGAGAAGTACGATG
GTTAAGCAGGCGAAAGAAAATTGTGACATACATCCCTGAGCGGCTGCATAAAACGTAGTACCTCTGAAGATACAGAGATAATTGTA
AATTACGGGGTAAATGCATCGCTGATACTATTTTGACAGGACTCTGTATTTTCTGGCTTTGATTATAGCGTTTCGGTTAATCAGTG
AAGGTATTACAGGAAAAGTGTGAGTAAGAGGAGCTATTTTGTTTCGTCAGGATGCTTTAGAAAACAGAAAAATGAAGTGGCAGGGA
CGGGCAATATTACTTCCCGGAATACCACTATGGTTAATCATGCTGGGAAGCATTGTGTTTATTACGGCATTTCTGATGTTCATTAT
TGTTGGTACCTATAGCCGCCGTGTTAATGTCAGTGGTGAGGTCACAACCTGGCCAAGAGCTGTCAATATATATTCAGGTGTACAGG
GATTTGTTGTCAGGCAATTTGTTCATGAAGGGCAGTTGATAAAAAAAGGGGATCCTGTTTATCTGATTGACATCAGTAAAAGTACA
CGTAGTGGTATTGTCACTGATAATCATCGGCGGGATATAGAAAATCAGCTGGTTCGTGTGGACAACATTATTTCCCGTCTGGAAGA
AAGTAAAAAAATAACGTTAGATACCCTGGAAAAACAACGTCTGCAATACACAGATGCGTTTCGTCGCTCATCAGATATTATACAGC
GTGCAGAGGAAGGGATAAAAATAATGAAAAACAATATGGAGAATTACAGAAACTATCAGGCAAAAGGGCTGATTAATAAAGATCAG
TTAACTAACCAGGTGGCATTATATTATCAGCAACAAAACAATCTTCTCAGCCTGAGCGGACAGAACGAACAGAATGCCCTGCAGAT
AACCACTCTGGAGAGTCAGATTCAGACTCAGGCTGCAGATTTTGATAACCGTATCTACCAGATGGAACTGCAACGGTACGAGTTAC
AGAAAGAACTGGTTAACACTGATGTGGAGGGCGAAATTATTATCCGGGCGTTGACTGACGGGAAAGTTGACTCCCTGAGTGTCACT
GTCGGGCAAATGGTCAATACCGGAGACAGCCTTCTGCAGGTTATTCCTGAGAACATTGAAAACTATTATCTTATTCTCTGGGTCCC
AAATGATGCTGTTCCTTATATTTCGGCTGGTGACAAAGTGAATATTCGTTATGAAGCCTTTCCGGCAGAAAAATTTGGGCAGTTCT
CTGCTACGGTTAAAACTATATCCAGGACTCCTGCGTCAACACAGGAAATGTTGACCTATAAGGGTGCACCACAGAATACGCCGGGC
GCCTCTGTTCCCTGGTATAAAGTCATTGCGATGCCTGAAAAGCAGATTATCAGATATGACGAAAAATACCTCCCTCTGGAAAATGG
AATGAAAGCCGAAAGTACACTATTTCTGGAAAAAAGGCGTATTTACCAGTGGATGCTTTCTCCTTTCTATGACATGAAACACAGTG
CAACAGGACCGCTCAATGACTAACGGGAGTTTCAGACAAATTATAAATCAGCTTGATATGCGCTGGCGACGTCGTGTTCCGGTTAT
TCATCAGACGGAGACCGCTGAATGTGGACTGGCCTGCCTGGCAATGATATGCGGTCATTTTGGTAAGAATATTGACCTGATATCTC
TTCGCCGGAAGTTTAATCTCTCGGCCCGTGGAGCAAACCTTGCAGGAATCAATGGAATAGCGGAGCAGCTGGGGATGGTCACCCGG
GCTCTTTCACTGGAGCTGGATGAACTTGGTGCCCTCAAAATGCCGTGTATTCTCCACTGGGATTTCAGTCACTTTGTCGTGCTGGT
CAGCGTAAAGCGTAACCGTTATGTACTGCATGATCCGGCCAGAGGCAGAAGATATCTCGGTCGGGAGGAAATGAGCCGGTATTTTA
CGGGCATTGCACTTGAGGTCTGGCCTGGAAGTGAATTCCTGGCGGAAACCCAGCAGATCCGCATAAGTCTCCGTTCACTGATTAAC
AGTATTTACGGTATTAAAAGAACACTGGCGAAAATTTTCTGTCTGTCAGTTGTAATTGAAGCAATCAATCTGGTAATGCCGGTGGG
GACTCAGCTGGTTATGGATCATGCGATTCCGGCGGGGGACAGAGGGCTGCTGACGCTTATTTCTGCTGGCCTGATGTTCTTTATAT
TGCTCAGGGCCGCGGTGAGTATGCTGCGTGCATGGTCCTCACTGGTTATGAGCACGCTCATCAATATACAGTGGCAGTCGGGTCTG
TTTAACCATCTTCTCAGACTGCCGCTGGCGTTTTTTGAACGCCGTAAATTAGGTGATATCCAGTCGCGTTTTGGCTCCCTTGACAC
TTTGAGGGCCACCTTTACCACCTGTGTGGTTGGGGCAATCATGGACAGTATTATGGTTGTGGGTGTTTTTGTGATGATGCTGTTAT
ATGGAGGATATCTTACCTGGATAGTGCTCGGTTTTACCATGGTTTACGTTCTTATTCGTCTGGTGACATACGGCTATTACCGGCAA
ATATCGGAAGAAACTCTTGTCAGGGGGGCCCGGGCCAGCTCCTATTTTATGGAAAGCCTGTATGGTATTGCCACGGTAAAAATCCA
AGGTATGGCTGGGATCCGGGGAACACACTGGCTTAACCTGAAAATAGATGCGATCAATTCAGGTATTAAGTTAACCAAGATGGATT
TGCTCTTCGGGGGGATAAATACTTTTGTTGCCGCCTGTGATCAGGTGGCGATTTTATGGCTGGGTGCAAGCCTTGTGATCGATAAT
CAGATGACAATAGGGATGTTTGTGGCATTTGGTTCTTTTCGTGGGCAGTTTTCGGATCGGGTTGCTTCGCTGACCAGTTTTCTTCT
TCAACTGAGAATAATGAGTCTGCATAATGAGCGCATTGCAGATATTGCACTACATGAAAAGGAAGAAAAGAAACCGGAAATTGAAA
TCGTTGCTGACATGAGCCCGGTTTCACTGGAAACCACTGATTTAAGCTACCGGTATGACAGCCAGTCAGCACAGGTATTCAGTGGT
CTGAATTTGTCTGTGGCTCCGGGAGAAAGTGTGGCTATAACTGGTGCCTCCGGTGCCGGAAAAACCACATTAATGAAAGTATTATG
TGGACTGTTTGAACCAGATAGTGGAAAAGTACTGGTTAATGGCACGGATATACGTCAACTTGGAATAAATAATTATCACCGTATGA
TAGCCTGTGTTATGCAGGACGACCGGCTATTTTCAGGATCAATTCGTGAAAATATCTGTGGGTTTGCAGAAGAAACAGACGACGAA
TGGATGACAGAATGTGCCAGAGCAAGTCATATTCATGATGTGATAATGAAAATGCCAATGGGGTATGAAACGTTAATAGGTGAACT
GGGGGAAGGTCTTTCCGGCGGTCAAAAACAGCGTATATTCATTGCCCGAGCTTTATACCGGAAACCTGGAATATTATTTATGGATG
AGGCTACAAGTTCTCTTGATACAGAAAGTGAACGTTTCGTGAATGCTGCCATAAAAAAAATGAATATCACCCGGGTGATTATTGCA
CACAGAGAAACTACGTTGAGAACTGTTGACAGGATTATTTCTATTTAAAATGAGGCCCTTTAACTGTGTGGATGGGACGATCGTTG
ACAGCTAGCTCAGTCCTAGGTATAATGCTAGCCTGAAACGGCTGCCGGTTAATAAGGAGGTACAACATGCGAAAACGTATTCTTTT
TATTGGCCCACCGCTGTACGGTTTGTTATACCCATTGATTTCTCTGGCTCAGGCCTTTCGTGTAATCGGACATGATGTAGTAATTA
GTAGTGCTGGCAAATTCGCGAATAAAGCAGCAGAAGCTGGACTGGTTGTTTTTGATGCAGTTCCAGGTTTAGATTCAGAGGCTGGA
TATCGCCATCAGGAAGAGTTGAGGAAAAAAAGTAATATTATTGGTCATTTCTCTTTTTTTAGCGATGAAATGGCAGATAACCTCAT
CGATTTTGCAGGAAAATGGAGGCCAGATTTAATAGTCTATCCCCCGCTTGGTCCGGCAGGCCCATTGGTTGCTGCTAAATATAGAA
TTCCTTCAGTGATGCTGGCTGTTGGATTCGCGCATACATCTGCCCATATTCAGATGTTAAACCGTTCTTTAAGCAATGCTTACAGG
CGGCATGGAGTCAGCGGTCCACTATGTGATTTAGCATGGATTGATGTTGCTCCCCCAAGTATGAGCATTCTTAAAAATGCTGAAGA
ACCGGTTATCTCAATGAGATATATTCCTTATAACGGAGGTGCTGTAAAGGAAACATGGTGGGACAGGGATTCTGATCGAAAACGTT
TACTCATCAGCCTTGGCACTGTAAAACCAATGGTTGATGGTCTGGAGCTGATTTCATGGGTTATGGATTCTGCAAATGAAGTTGAT
GCTGATATCATTTTGCAACTTGCAATAAATGCTCGTACTGGATTACGAAAACTACCATCAAATGTACGTCTGGTTGACTGGATACC
TATGGGTGTATTCCTTAATGGAGCTGATGGATTTATTCATCATGGTGGCGCAGGTAATACCCTGACAGCGTTGTATAGTGGGATAC
CACAGATTGTGTTTGGCGAAGGTGCAGATCGCTCTGTTAATGCAGAAATTGTTGCGATGCGTGGGTGTGGGATTATTCCGGACAAG
CATGGACTGACCAGTGATTTGGTAAATCGCCTGCTTTATGATGATTCACTACGCTTCTGTTCAGATCAGGTAGCCGCTGAAATGGC
TGAACAACCCAGTCCTGCAGAGATCGCAGAGGTTTTGATGAGAAAATTAAAAAACAACGGGAAATAAGAACACGAAGACATGGCCT
TATCTAAAACAGACCATACGTTATCAATACCAGAAAAATATATTGTTATCGGTATAAATAAAAACAACATTGATAAGAGATACATT
CTAATTTTCATTTTTGTAAAATTTCCTGTACCACGTTGATCTACTTATTCCTAAAGAAATCCATTCTCCATCTCTAACTTTCGGCC
TTCCACCACCAGAGCTTTTTTTTCCACGTTGACGCTGAATTTCAGAAGTATGTGTTTGTTTAACATACTCTTCAAAGCCAAGCTCT
GTAAGGTTCTTACTTGTCCACTTAGCCACACTTTTAGCAATTCCCATGACTTCGTTCTCGTCTAAAGGTGCGGAGAACTGCAGGTT
GTAGGCTTTAGCGCGTTCAATGCAGGCTTGTAGCCATTGGTCATACTGCGGCCAGCCTTGGCGGATAGCGCGGTAAGCCCACTTGC
GGGTTTTATCGAAGAGGGTGCAGTTACGGCCTAAACCGTAGTCCGGCAGGATTTCGCGGTCATTGGCTGCGCCAAGGTCGAGGTAA
TCGGCTAACCAGTCAAGGGTATAGAGCTCTGGCTGCCAGACGGTGATCTGCCAGTGCAGGTGGTTCGGATTCTTGCAAATTAGCCC
TGAATACCCCGCATCTGCGCCCAATTTTTTACGCAGCGCATTCTCGATGGCGGCGGCGTATTTAAGGGGGGCAGCTCGACCATCCG
GCGCGGTACGTACCGCCGTATGCAAGGCATACAACAGGTGAGCATGTCCGTTCTCGGGGTTTTTGATGGTGAGTGTGGGCGCAGGT
GCCCCCAGATCGGCCCAATCAATCGCGGCTCCGGCTCTGTCCACGTCAAAGCAAAGCCAGTACATGGCGTGAGGCTGATTAAACTG
GATGTATTTTGCGAGGAGAGCACGCTCTTTACCGGCAATGCGAACACCAAACTGTAAATCATCGGAGAAGTACGGCTTGTGGGGTA
ACCGGTCGTTAAAAAGCGTTAAAGCCTGATTATCCAAGGCTCCCAGCCTTATGGCGGGGCTGTTGTTTTGCACGCTGCATGTGCTA
ATATCCTTTCTAGGTTTCGACCTAGCCCTGAATGTCATGTCCGCTCGCCAAAGTAGAGCATGATTTCGGGGCTTTGTTTTTTCTGC
CACTAAGTTACACCTCAACAACGGTTTTTGTCATCCCCGACAATCCGTTATTCCTGCTTGTTCTCGCACGGCTTTACGCTCATACT
ACTTCTTGTAGATACACTTGTCACTACATCAAGAGGTGAGATGATGGCCACGATTAATATTCGGATCGATGACGAGCTGAAAAGCC
GCTCTTATGCCGCACTGGAAAAGCTGGGCGTAACGCCGTCCGAGGTTCTGCGCCAAACACTGGAATATGTGGCCCAAAGCGGACGT
TTGCCGTTCCAGCAGGTTTTGCTGACCGAGGATGATGCCGATTTGATGGCTATCGTTCGGGATCGTCTGGAAAACCCACAGGCGGG
CGTAAAGGTGTCACTGGATGAGCTATAACCTTGAATTTGATCCCCGAGCCCTGAAGGAATGGCGCAAGCTCGGGGATGATGTCCGT
CTGCAGTTCAAGAAAAAACTCGAGCAGGTTCTACAACACCCGCGGATCGATAAAAATCGCCTGCGAGAGCTGCATGACTGCTACAA
AATCAAGCTCCGTGCATCCGGTTATCGCTTGGTCTATCAGGTTCGCGATCAAACCATTACGGTATTCGTGGTGGCGGTCGGTAAGC
GCGAGCGTTCTGCCGCTTACGATGCGGCCGATAAACGCTTATAAACTCATGCCGTGACCGCGAGAATACCGCTGTTCGTGCCGCTT
GGCTAATTGCTCAAGCGGCGCACTGTTCTGTTTAAGCTCTCGACTTGCTGCGCCAAGCCGGTGACTTGCATGCTCAAGGCCTGACA
AACCCTGTCGTTTTTCGCCAACTCCTGCGAGGTAACCTCGAACATGCGCTGTAAGTTGGCGTAGCTGTCCTGCCACGCTTGCTGCT
GTTGTTCGTAGTGCCTCTGTAAGCTCTCTAATGCGCTCAGAAGCTGCTGCTCCATTTCGGTCATGAATCTCTTCACCCTGATAGAT
AAAACCGCCCAGAATCGATTCTGTGGCGTCTGATGAGGTTATTTGGCGCTGTACTTGATGACCTGACGATGTTGAGCGTTCTTGTA
CTCGTCGATCTTCTTCGCCCCCTGCGGAAGGATCAGGTAATACACGCTCTTGTTCTTGGAATCGTGAATTATCGATACGCCGGCTC
CGGTCTGGCTCTTTAAGTCCTGCAGGATCTGGCTCTGCTCGCTGATTTCGTTCTGGCGTTCGACCACGATAGTCCCGAGATACCAA
GCTACTCCAATCAATATCGCAAACAGGATCCCACTTAATGCCAGGCTGTACAGCCATGTCATTCCGACTAAGCGGTGTATCTGTTT
TAGCTGGCTGTCGTTCTCTTCTTGGATAGCGGTCTGTATGTTCCCTGAGCTTAATTTCAAGGCCTCGGTGATACGTTTCTCGTGCT
TCTCGAATGCGTTCGCGACGCTCGTTGCGGTAGTCTTGGCTTGCTGCTTCGATTTGCTCTCGAACTCCCGCGCTAAATTTAAAATC
TCGCTCATACAGCACTCCTTTTAAGCGAATATTCGGGCCACCTGCCGGATCAGCAATACTGATACTGGATTTGGTTTCCCGTACGA
CCGACAATCCGGCATCGGTAAGGTGGGAAATCACCCCTTTACGATCCGTAATTTCTCCCTGCTCAATCAAGCTGATTAGCCCTTTG
GTAATGGCTTCCGCTGCCTGCTGTTTGTTGCGAGGAAGGTCATTAGAGGGGGTTAATGCTCGGCGATTAGCAGGGTCATTCGGGTC
GCGTAACCCAAGCCGGTCATTGGTGAGGGTTTGCCATGCGTTAACACGAGGCCGGTCAGCCCGATCAAAGTAAGGTTGTAGCCGTT
TTCCGCTCTGCAATTCGATGTTCGGGATAACAAAATTCAATTCAAGACGCCCTTTGTCCTGATGTTGAACCCAGAGGCAGGCATAC
TGGTCTTTATCTAGACCGGTCATCAATGTCTGCTCCCATTCATCCATCAATCGCTGCTTTTCGCCTTCGGGTAAATCACTCTCCTG
AAAAGAGAGCACGCCAGAGGTATAAGTTCGGGCAAATTCGCAGCCATCAATCAGCTCTTTGACGTGCTCAGGGTTACCCCGTAACA
CCGTCGCTTGTTCGCGCTGACGATCAGGGCCCAGAAGGTAATCGACAGGACCACTCCCGCCACCGGCACCACGACCATGAATCTTA
ACGATCACGATGTTGCTCCAGCAGTTCGGCAAGATGTTGGTCAATGCTATTGAGCACCGCTAACAACGACACCCGTTCTTGCGGCG
GTAAGCCATGCTGATTCAAGTAACGGGCTATTTGATTGAGGTTATTACCGATCCCGCTGACCTGACGTAACAAGGTCGGGTCTACG
GTAGGTAGCGGACGACGCCGAGCTGTACGCGATTCGCCTAAGCCAACGGCTCGTAACCACTCGGCCAAATGCTTACGGTCACAGCG
TTCAAGTAGCCGCTGATGCTCCGCTTCGGTGAGTCTGA

In some embodiments, the self-retaining vector is backbone of a native vector of the microorganism. In some embodiments, the self-retaining vector comprises one or more genetic modifications of the native vector of the microorganism. In some embodiments, the microorganisms described herein includes a native self-retaining vector in its wild-type species, which is deleted during counterselection. The genetically-modified self-retaining vector is then introduced in the microorganism during genetic modification. Therefore, in some embodiments, the genetically-modified microorganism described herein comprises a deletion of the native vector and the introduced self-retaining, genetically-modified vector.

In some embodiments, the genetically engineered microorganism described herein retains the self-retaining vector without antibiotic selection.

Examples of bacterial species that can be used as engineered microorganisms include, but are not limited to: Acinetobacter baumannii, Enterobacter cloacae, Escherichia coli, Klebsiella oxytoca, Klebsiella pneumoniae, Proteus mirabilis, Pseudomonas aeruginosa, Salmonella Typhimurium, Salmonella Typhi, Serratia marcescens, Shigella flexneri, and Staphylococcus aureus.

### Methods of Treating Bacterial Infections

Microcins (e.g., MccH47 or MccI47) have been shown to be active to inhibit various bacteria, e.g., gram-negative bacteria. As used herein, the term “gram-negative bacterium” refers to a bacterium that do not retain the crystal violet stain used in the Gram staining method of bacterial differentiation. Gram-negative bacteria include, e.g., proteobacteria, cocci, bacilli, etc. The proteobacteria are a major group of gram-negative bacteria, including Escherichia coli (E. coli), Salmonella, Shigella, and other Enterobacteriaceae, Pseudomonas, Moraxella, Helicobacter, Stenotrophomonas, Bdellovibrio, acetic acid bacteria, Legionella etc. Gram-negative bacteria also include, e.g., the cyanobacteria, spirochaetes, green sulfur, and green non-sulfur bacteria. Medically relevant gram-negative cocci include, e.g., Neisseria gonorrhoeae, Neisseria meningitidis, and Moraxella catarrhalis, Haemophilus influenzae. Medically relevant gram-negative bacilli include a multitude of species.

Some of them cause primarily respiratory problems (Klebsiella pneumoniae, Legionella pneumophila, Pseudomonas aeruginosa), primarily urinary problems (Escherichia coli, Proteus mirabilis, Enterobacter cloacae, Serratia marcescens), and primarily gastrointestinal problems (Helicobacter pylori, Salmonella enteritidis, Salmonella Typhi). Gram-negative bacteria associated with hospital-acquired infections include, e.g., Acinetobacter baumannii, which cause bacteremia, secondary meningitis, and ventilator-associated pneumonia in hospital intensive-care units.

In some embodiments, the composition and the methods as described herein can be used to treat gram-negative bacterial infections. In some embodiments, the bacterial infection is carbapenem-resistant Enterobacteriaceae infection, Klebsiella oxytoca infection, Klebsiella pneumoniae infection, Campylobacter infection, extended spectrum Enterobacteriaceae (e.g., E. coli, Salmonella, Shigella and Yersinia) infections. Thus provided herein are methods for treating a subject comprising administering an effective amount of a composition as described herein to a subject in need of such treatment.

The methods described in the present disclosure are effective for treating bacterial infections in a variety of subjects including humans and animals, such as laboratory animals, e.g., mice, rats, rabbits, or monkeys, or domesticated and farm animals, e.g., cats, dogs, goats, sheep, pigs, cows, horses, and birds, e.g., chickens and turkeys.

Healthcare providers can identify subjects in need of treatment for bacterial infections using their experience and judgment, which can be based on subjective (e.g., based on the healthcare provider's opinion) or objective (e.g., measurable by a test or diagnostic method) information. As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

The present disclosure provides methods of inhibiting or reducing the risk of bacterial infections and for treating bacterial infections. As used herein, the term “reducing the risk” refers to reducing the probability of developing a disorder or condition in a subject, who does not have, but is at risk of, or susceptible to, developing a disorder or condition.

In some embodiments, the compositions, e.g., genetically engineered microorganisms, can be administered to a subject with some other known treatments for bacterial infections. For example, the genetically engineered microorganisms can be used in combination with an antibiotic therapy, such as metronidazole, vancomycin, bacitracin, and/or teicoplatin. In some embodiments, the genetically engineered microorganisms are administered to the subject after the subject have received an antibiotic therapy. In some embodiments, the genetically engineered microorganisms are administered to the subject before the subject has received an antibiotic therapy. In other embodiments, the genetically engineered microorganisms are administered to the subject when the subject is under an antibiotic therapy.

In some embodiments, the genetically engineered microorganisms can be administered to a subject with alkaline phosphatase. These methods involve administering to the subject a composition including the genetically engineered microorganisms and an amount of an alkaline phosphatase effective to increase the number of commensal bacteria in the gastrointestinal tract, wherein alkaline phosphatase decreases the number of pathogenic bacteria in the gastrointestinal tract, or increases the number of commensal bacteria and decreases the number of pathogenic bacteria in the gastrointestinal tract, thereby modulating gastrointestinal tract flora levels in the subject. The alkaline phosphatase composition, and the methods of use is described in WO 2010/025267, which is incorporated by reference in its entirety.

### Methods of Treating Dysbiosis

The compositions and the methods as described herein can be used to treat and/or reduce the risk of dysbiosis and its associated diseases. Thus provided herein are methods for treating a subject comprising administering an effective amount of a composition as described herein to a subject in need of such treatment.

Dysbiosis is a term for a microbial imbalance or maladaptation on or inside the body. As used herein, the term “intestinal dysbiosis” refers to microbial imbalance in intestines. Dysbiosis is most commonly reported as a condition in the gastrointestinal tract, particularly during small intestinal bacterial overgrowth (SIBO) or small intestinal fungal overgrowth (SIFO). It has been reported to be associated with various diseases, such as periodontal disease, inflammatory bowel disease, chronic fatigue syndrome, obesity, cancer, bacterial vaginosis, and colitis.

The methods described in the present disclosure are effective for treating dysbiosis in a variety of subjects including humans and animals, such as laboratory animals, e.g., mice, rats, rabbits, or monkeys, or domesticated and farm animals, e.g., cats, dogs, goats, sheep, pigs, cows, horses, and birds, e.g., chickens and turkeys.

Healthcare providers can identify subjects in need of treatment for dysbiosis using their experience and judgment, which can be based on subjective (e.g., based on the healthcare provider's opinion) or objective (e.g., measurable by a test or diagnostic method) information. As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

The present disclosure provides methods of inhibiting or reducing the risk of dysbiosis and for treating dysbiosis. As used herein, the term “reducing the risk” refers to reducing the probability of developing a disorder or condition in a subject, who does not have, but is at risk of, or susceptible to, developing a disorder or condition.

In some embodiments, the genetically engineered microorganisms can be administered to a subject with some other known treatments for dysbiosis.

### Methods of Administration

The therapeutic methods disclosed herein (including prophylactic treatments) generally include administration of a therapeutically effective amount of a composition comprising the genetically engineered microorganisms to a subject in need thereof. Such treatment will be suitably administered to subjects, particularly humans, suffering from, having, susceptible to, or at risk for a disease, disorder, or symptom of bacterial infection and/or dysbiosis. Determination of those subjects who are “at risk” can be made by any objective or subjective determination by a diagnostic test or opinion of a health care provider.

A subject is effectively treated when a clinically beneficial result ensues. This may mean, for example, a resolution of the symptoms associated with bacterial infection and/or dysbiosis, a decrease in the severity of the symptoms associated with bacterial infection and/or dysbiosis, or a slowing of the progression of symptoms associated with bacterial infection and/or dysbiosis.

The compositions can also include a pharmaceutically acceptable carrier. The term “pharmaceutically acceptable” refers to molecular entities and compositions that do not produce an adverse, allergic or other untoward reaction when administered to a subject. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Compositions comprising the genetically engineered microorganisms can be administered to a subject through many different routes, e.g., by endoscopy, by enteroscopy, by colonoscopy, by a nasoduodenal catheter, by enema, or by oral administration. In the case of oral administration, the composition can be delivered in a capsule or pill form, e.g., for intestinal delivery. In some embodiments, the composition is in a capsule form, e.g., packaged in gelatin capsules.

The present disclosure also provides a food composition comprising the genetically engineered microorganisms. In some embodiments, the food composition comprises carbohydrates such as, but not limited to, starches such as are contained in rice flour, flour, tapioca flour, tapioca starch, and whole wheat flour, modified starches or mixtures thereof.

In some embodiments, the compositions including the genetically engineered microorganisms are in the form of a liquid, and thus can be used as a beverage. In some embodiments, the beverage composition comprising the genetically engineered microorganisms is naturally sweetened. Suitable natural sweeteners include, but are not limited to, sugars and sugar sources such as sucrose, lactose, glucose, fructose, maltose, galactose, corn syrup (including high fructose corn syrup), sugar alcohols, maltodextrins, high maltose corn syrup, starch, glycerin, brown sugar and mixtures thereof.

In some embodiments, the food or beverage compositions include milk or milk-derived product, e.g., yogurt. In some embodiments, a stabilizer may be combined with the milk-derived product. Combining a stabilizer with the milk-derived product may thicken the milk-derived product. In some embodiments, a stabilizer can be combined with the milk-derived product following completion of microorganism culture. The stabilizer can be selected from, as examples, gums, salts, emulsifiers, and their mixtures. Gums can be selected from, as examples, locust bean gum, xanthan gum, guar gum, gum arabic, and carageenan. In some embodiments, salts include, but are not limited to, sodium chloride and potassium chloride.

### Dosage

The compositions can be formulated in a unit dosage form, each dosage containing, for example, from about 0.005 mg to about 2000 mg of the genetically engineered microorganisms. The dosage scheduling can be approximately once per week, twice per week, three times per week, or four times per week. In some embodiments, the compositions can be administered to a subject every day, every other day, every three days, every four days, every five days, every six days, or once per week. A person skilled in the art can refine the dosage scheduling as needed.

The phrase “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. When referring to these pre-formulation compositions as homogeneous, the active ingredient is typically dispersed evenly throughout the composition so that the composition can be readily subdivided into equally effective unit dosage forms.

The compositions can be formulated in a unit dosage form, each dosage containing, for example, from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the genetically engineered microorganisms.

### Kits

The present disclosure also provides kits of the genetically engineered microorganisms. In some embodiments, the kit includes a sterile container which contains a therapeutic or prophylactic composition having the genetically engineered microorganisms. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The kit can also include instructions, e.g., information about the use of the composition for treating a bacterial infection. The kit can further contain precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The invention is further described in the following examples, which do not limit the scope of the invention described in the claims.

### EXAMPLE 1: Microcin MccI47 Selectively Inhibits Enteric Bacteria and Reduces Carbapenem-Resistant Klebsiella pneumoniae Colonization In Vivo when Administered Via an Engineered Live Biotherapeutic

The human gut microbiome is a densely populated ecosystem, where the bacterial symbionts are in constant competition for survival (Foster K R et al. The evolution of the host microbiome as an ecosystem on a leash. Nature. 2017; 548(7665):43-51). A major strategy employed by bacteria to outcompete their neighbors is the production of highly specific antimicrobial peptides, so called bacteriocins, that target closely related species or strains (Granato E T et al., The Evolution and Ecology of Bacterial Warfare. Current Biology. 2019; 29(11):R521-R37). While small molecule antimicrobials, such as penicillin (Fleming A. On the Antibacterial Action of Cultures of a Penicillium, with Special Reference to their Use in the Isolation of B. influenzae. Br J Exp Pathol. 1929; 10(3):226-36), have been exploited for decades as traditional antibiotics, antimicrobial peptides as therapeutic agents have just recently gained widespread interest as potential treatments of MDR pathogens (Fjell C D et al. Designing antimicrobial peptides: form follows function. Nature Reviews Drug Discovery. 2012; 11(1):37-51; doi: 10.1038/nrd3591; and Jenssen H et al., Peptide antimicrobial agents. Clin Microbiol Rev. 2006; 19(3):491-511). Siderophore antimicrobial peptides (sAMPs), including class IIb microcins, are ribosomally synthesized and post-translationally modified peptides (RiPPs) consisting of an antimicrobial peptide (AMP) linked to an iron-chelating molecule, a siderophore (e.g., enterobactin) (Telhig S et al., Bacteriocins to Thwart Bacterial Resistance in Gram Negative Bacteria. Frontiers in Microbiology. 2020; 11(2807)). Several studies have pointed to class IIb microcins as possible players capable of modulating the colonization dynamics of pathogenic Enterobacteriaceae in the GI tract (Sassone-Corsi M et al. Microcins mediate competition among Enterobacteriaceae in the inflamed gut. Nature. 2016; 540(7632):280-3; Gargiullo L et al., Gut Microbiota Modulation for Multidrug-Resistant Organism Decolonization: Present and Future Perspectives. Front Microbiol. 2019; 10:1704; Palmer J D et al., A Class IIb Microcin with Potent Activity Against Multidrug Resistant Enterobacteriaceae. ACS Infectious Diseases. 2020; 6(4):672-9; and Palmer J D et al, Bucci V. Engineered Probiotic for the Inhibition of Salmonella via Tetrathionate-Induced Production of Microcin H47. ACS Infect Dis. 2018; 4(1):39-45), however, to date only three of them (MccM, MccH47, MccE492) have been characterized in detail (Vassiliadis G et al., Isolation and characterization of two members of the siderophore-microcin family, microcins M and H47. Antimicrob Agents Chemother. 2010; 54(1):288-97; Azpiroz M F et al., Involvement of enterobactin synthesis pathway in production of microcin H47. Antimicrob Agents Chemother. 2004; 48(4):1235-41; Lavińa M et al., Microcin H47, a chromosome-encoded microcin antibiotic of Escherichia coli. Journal of Bacteriology. 1990; 172(11):6585-8; doi: 10.1128/jb.172.11.6585-6588.1990; and Orellana C, Lagos R. The activity of microcin E492 from Klebsiella pneumoniae is regulated by a microcin antagonist. FEMS Microbiology Letters. 1996; 136(3):297-303). MccI47 from E. coli strains H47 and CA46 has been identified based on sequence homology to known sAMPs, but it has never been overexpressed heterologously, purified, and no data demonstrating antimicrobial activity have been presented before (Poey M E et al. Comparative analysis of chromosome-encoded microcins. Antimicrobial agents and chemotherapy. 2006; 50(4):1411-8). In this study we first leverage our recently established pipeline for sAMPs purification to provide the first ever characterization of MccI47 as a potent antimicrobial against multiple Enterobacteriaceae. We then perform rational genetic manipulation of the probiotic E. coli Nissle 1917 (EcN) by engineering one of its two native plasmids to produce MccI47 without the need of any selection and demonstrate the ability of this new EcN strain in reducing CR K. pneumoniae abundance in vivo.

The gastrointestinal (GI) tract is the reservoir for multidrug resistant (MDR) pathogens, specifically carbapenem-resistant (CR) Klebsiella pneumoniae and other Enterobacteriaceae, which often lead to the spread of antimicrobial resistance genes, severe extraintestinal infections, and lethal outcomes. Selective GI decolonization has been proposed as a new strategy for preventing transmission to other body sites and minimizing spreading to susceptible individuals.

Here, we purify the to-date uncharacterized class IIb microcin I47 (MccI47) and demonstrate potent inhibition of numerous Enterobacteriaceae, including multidrug-resistant clinical isolates, in vitro at concentrations resembling those of commonly prescribed antibiotics. We then genetically modify the probiotic bacterium Escherichia coli Nissle 1917 (EcN) to produce MccI47 from a stable multicopy plasmid by using MccI47 toxin production in a counterselection mechanism to engineer one of the native EcN plasmids, which renders provisions for inducible expression and plasmid selection unnecessary. We then test the clinical relevance of the MccI47-producing engineered EcN in a murine CR K. pneumoniae colonization model and demonstrate significant MccI47-dependent reduction of CR K. pneumoniae abundance after seven days of daily oral live biotherapeutic administration without disruption of the resident microbiota.

This study provides the first demonstration of MccI47 as a potent antimicrobial against certain Enterobacteriaceae, and its ability to significantly reduce the abundance of CR K. pneumoniae in a preclinical animal model, when delivered from an engineered live biotherapeutic product. This study serves as the foundational step towards the use of engineered live biotherapeutic products aimed at the selective removal of MDR pathogens from the GI tract.

### Results

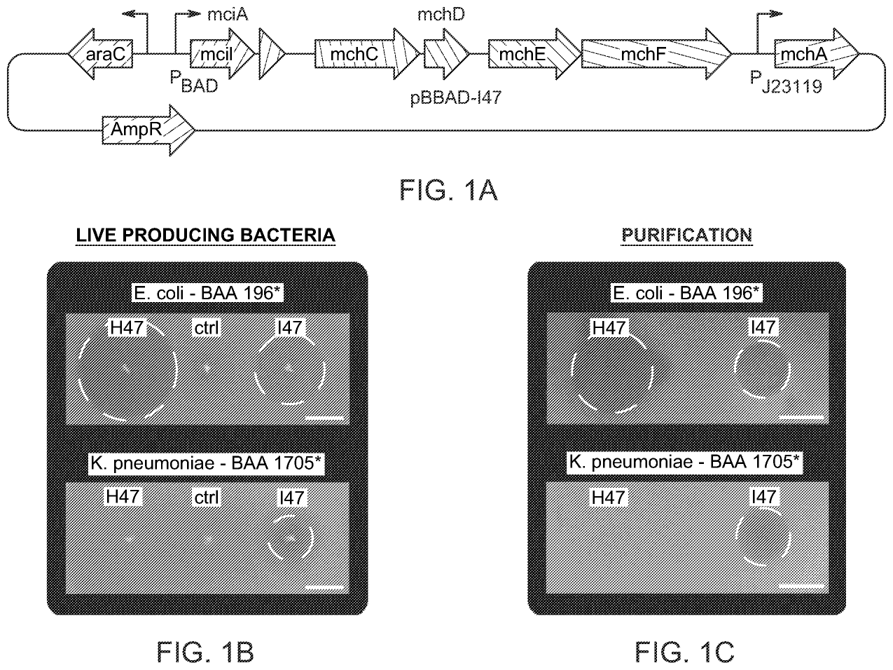

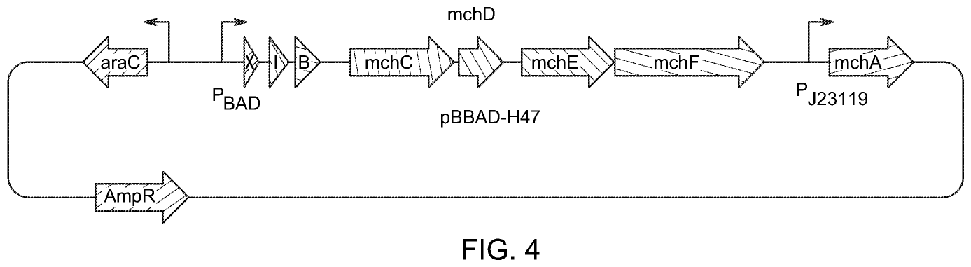

To expand the library of potent antimicrobials against MDR Enterobacteriaceae, we overexpressed MccI47 heterologously in E. coli and tested its inhibitory activity against a collection of enteric bacteria, including MDR clinical isolates. Specifically, we created a plasmid that contained the structural microcin (mciA) and immunity (mciI) gene under arabinose induction, followed by the genes necessary for posttranslational modification with monoglycosylated enterobactin (MGE) mchCDEFA (as in Palmer et al., ACS Infectious Diseases. 2020; 6(4):672-9) (FIG. 1A). Highlighting the differences in activity spectrum and efficacy, we directly compared the potency of MccI47 with MccH47 (FIG. 4), a well-characterized sAMP that has been used in previous proof-of-concept engineered biotherapeutics against Salmonella (Palmer et al., ACS Infectious Diseases. 2020; 6(4):672-9). Using static inhibition assays, we demonstrate that both microcins have strong inhibitory activity against extended-spectrum beta-lactamase (ESBL) producing E. coli (BAA 196). Intriguingly, MccI47 also inhibits the growth of CR K. pneumoniae (BAA 1705), while MccH47 has no effect (FIG. 1B). We purified MccI47 with a maltose-binding protein (MBP)-microcin fusion protein (Palmer et al., ACS Infectious Diseases. 2020; 6(4):672-9) and direct comparison of MccH47 and MccI47 in equal quantities reproduced the results obtained using the live-producing bacteria, highlighting MccI47's efficacy against CR K. pneumoniae (BAA 1705) (FIG. 1C). We next assessed the potency of MccI47 in minimum inhibitory concentration (MIC) assays against a panel of 20 different target strains (Table 1). Overall, MccI47's potency is comparable to that of commonly prescribed broad-spectrum antibiotics, while being highly specific for members of the Enterobacteriaceae family (Hoerr V et al. Characterization and prediction of the mechanism of action of antibiotics through NMR metabolomics. BMC Microbiol. 2016; 16:82). Compared to MccH47, MccI47 is approximately three-times more effective against various E. coli strains (0.2-0.6 μM), four-times more effective against Shigella flexneri (0.06 μM), and seven-times more effective against Salmonella Typhimurium (0.9-1.7 μM). More importantly, MccI47 but not MccH47 demonstrates activity against Enterobacter cloacae (19.5 μM), Serratia marcescens (15.1 μM), and Klebsiella pneumoniae (2.0-5.1 μM). Interestingly, neither of the two toxins showed activity against Klebsiella oxytoca or any tested bacteria outside of the Enterobacteriaceae family. We did not detect any difference in potency in targets carrying MDR genes, which is likely because the mode of entry of MccI47 into the target cells is independent of the resistance mechanisms of commonly used antibiotics (Baquero F et al., Microcins in Enterobacteriaceae: Peptide Antimicrobials in the Eco-Active Intestinal Chemosphere. Frontiers in Microbiology. 2019; 1(2261)). Taken together these data demonstrate the discovery of MccI47 as a new potent AMP with selective activity against several Enterobacteriaceae members, including a strong inhibitory effect against CR K. pneumoniae representatives.

TABLE 1
Results of Minimum Inhibitory Concentration (MIC) Assays
of purified MccI47 against bacterial isolates

| | | | MccI47 | | |
|---|---|---|---|---|---|
| | | | MIC | | MccH47[20] |
| Bacterial species | Strain | Origin | (μg/ml) | MIC (μM) | MIC (μM) |
| Acinetobacter | BAA-1790 | Human clinical | >138.8 | >19.6 | >113 |
| baumannii* | | isolate (sputum) | | | |
| Enterobacter | BAA-2341 | Human clinical | 138.4 | 19.5 | >113 |
| cloacae* | | isolate | | | |
| Escherichia coli | 25922 | Human clinical | 1.6 | 0.2 | 5.3 |
| | | isolate | | | |
| Escherichia coli* | BAA-196 | Human clinical | 4.4 | 0.6 | 1.8 |
| | | isolate | | | |
| Escherichia coli | DH5α | Laboratory | 2.3 | 0.3 | 1.1 |
| Klebsiella oxytoca* | 51983 | Human clinical | >197.3 | >27.8 | >113 |
| | | isolate (blood) | | | |
| Klebsiella oxytoca | 700324 | Human isolate | >197.3 | >27.8 | >113 |
| | | (bioMérieux, Inc.) | | | |
| Klebsiella | BAA-1705 | Human clinical | 36.1 | 5.1 | >113 |
| pneumoniae* | | isolate (urine) | | | |
| Klebsiella | BAA-2146 | Human clinical | 29.4 | 4.2 | >113 |
| pneumoniae* | | isolate | | | |
| Klebsiella | BAA-2342 | Human clinical | 16.4 | 2.3 | >113 |
| pneumoniae* | | isolate | | | |
| Klebsiella | BAA-2524 | Human clinical | 14.7 | 2.0 | >113 |
| pneumoniae* | | isolate | | | |
| Proteus mirabilis | 29906 | Human clinical | >197.3 | >27.8 | 5.3 |
| | | isolate (urogenital) | | | |
| Pseudomonas | PA14 | Human clinical | >138.8 | >19.6 | >113 |
| aeruginosa | | isolate | | | |
| Salmonella | 19585 | Derived from LT2 | 8.0 | 1.1 | 8.6 |
| Typhimurium | | (natural source) | | | |
| Salmonella | 29630 | Derived from LT2 | 6.3 | 0.9 | 6.3 |
| Typhimurium | | (natural source) | | | |
| Salmonella | BAA-190 | Human clinical | 12.6 | 1.7 | 12.7 |
| Typhimurium* | | isolate | | | |
| Serratia marcescens | DB11 | Derived from | 107.0 | 15.1 | >113 |
| | | DB10 (Drosophila) | | | |
| Shigella flexneri | 2457T | Human clinical | 0.4 | 0.06 | 2.4 |
| | | isolate | | | |
| Shigella flexneri | M90T | Human clinical | 0.4 | 0.06 | 4.4 |
| | | isolate | | | |
| Staphylococcus | 27661 | Human isolate | >197.3 | >27.8 | >113 |
| aureus | | | | | |

Data in Table 1 include MDR Enterobacteriaceae in comparison to MccH47[20]. Asterisks indicate MDR strains.

Due to the potent inhibitory effect against K. pneumoniae observed in vitro, we hypothesized that MccI47 could be used as a novel narrow-spectrum antimicrobial to reduce CR K. pneumoniae colonization in the GI tract in a murine model. We therefore developed an engineered EcN strain with MccI47-production from a native, modified multicopy plasmid, allowing for long-term plasmid retention without the need of selection. We chose EcN as vehicle strain because it has been used in its wildtype form as a live probiotic supplement for several human diseases for over a century (Pradhan S et al., Nissle in Human Intestinal Organoids. mBio. 2020; 11(4):e01470-20), carries genomic islands that promote anti-inflammatory responses (Sonnenbom U et al., The non-pathogenic Escherichia coli strain Nissle 1917—features of a versatile probiotic. Microbial Ecology in Health and Disease. 2009; 21(3-4):122-58), and was found to be highly refractory to exogenous plasmid acquisition (Crook N et al. Adaptive Strategies of the Candidate Probiotic E. coli Nissle in the Mammalian Gut. Cell Host Microbe. 2019; 25(4):499-512.e8). EcN is used for the construction of genetically engineered bacteriotherapeutic agents which are being tested in multiple ongoing clinical trials (Charbonneau M R et al., Developing a new class of engineered live bacterial therapeutics to treat human diseases. Nature Communications. 2020; 11(1):1738).

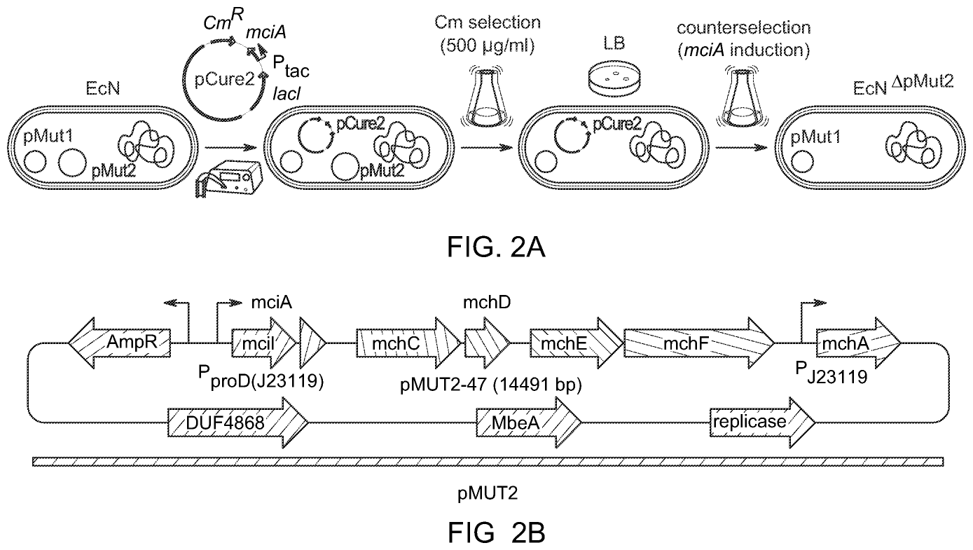

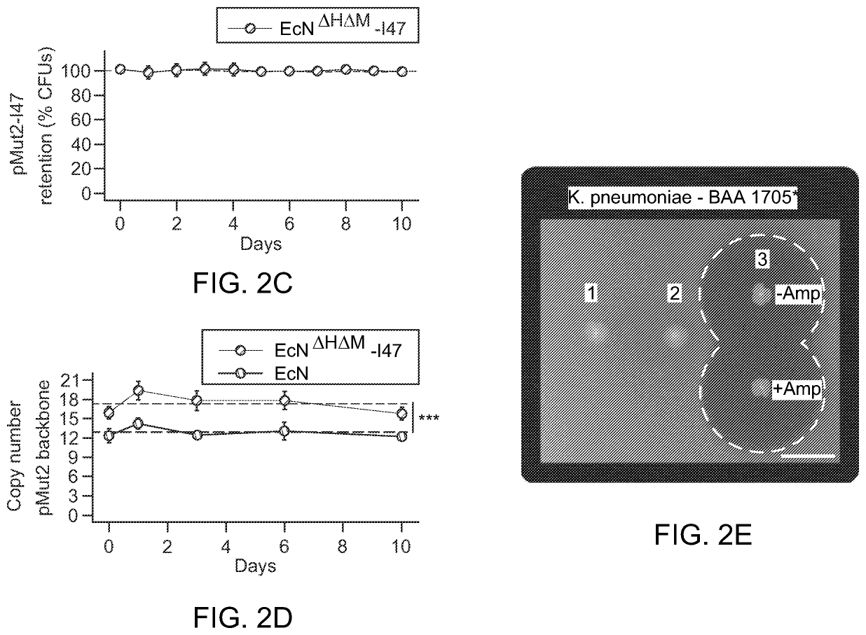

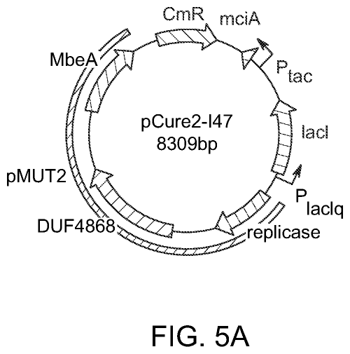

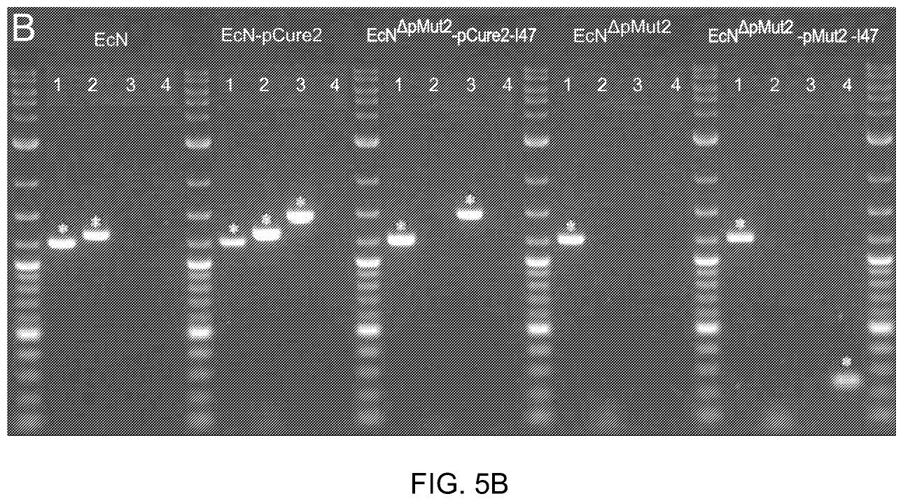

While recent studies relied on chromosomal integration (Isabella V M et al. Development of a synthetic live bacterial therapeutic for the human metabolic disease phenylketonuria. Nat Biotechnol. 2018; 36(9):857-64; and Leventhal D S et al. Immunotherapy with engineered bacteria by targeting the STING pathway for anti-tumor immunity. Nature Communications. 2020; 11(1):2739), we sought to maximize MccI47 output using a multicopy plasmid (Forkus B et al., Antimicrobial Probiotics Reduce Salmonella enterica in Turkey Gastrointestinal Tracts. Scientific Reports. 2017; 7(1):40695; and Kan A et al., Plasmid Vectors for in Vivo Selection-Free Use with the Probiotic E. coli Nissle 1917. ACS Synthetic Biology. 2021; 10(1):94-106) and developed an expression system using the native, self-retaining EcN plasmid pMut2 as backbone for constitutive heterologous protein expression. We first cured EcN from pMut2 by utilizing MccI47 (mciA) in a counterselection approach (FIG. 2A). For the curing plasmid pCure2-I47 (FIG. 5A) we employed pMut2 as backbone for plasmid competition, while harboring a chloramphenicol resistance, and placing mciA without its signal peptide for secretion and the protective immunity gene under isopropyl β-d-1-thiogalactopyranoside (IPTG) induction. Strong antibiotic selection allowed us to first displace pMut2, before inducing mciA expression and thus cell death in cells harboring pCure2-I47 (FIG. 2A).

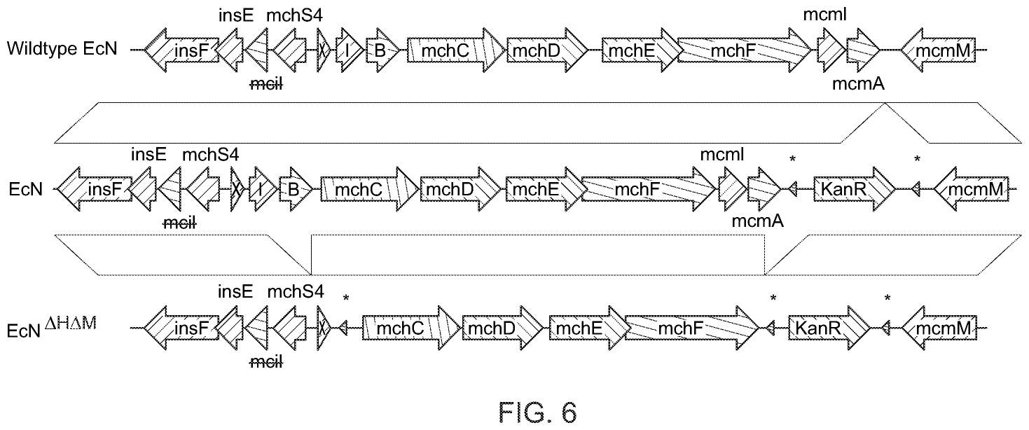

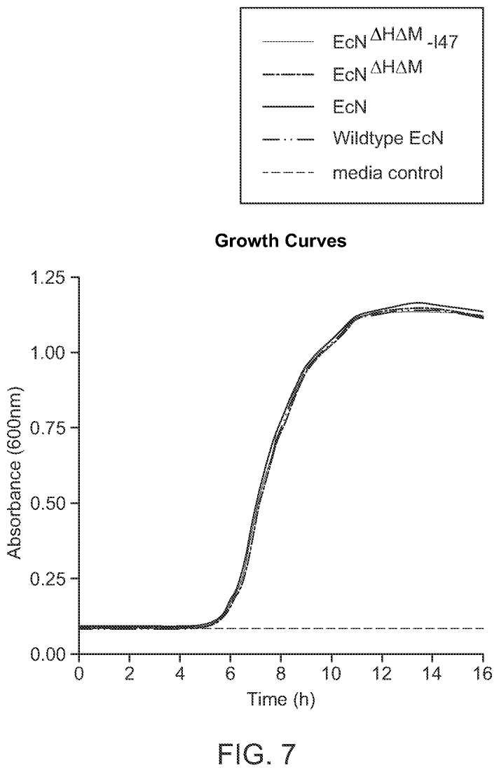

The resulting cells cured of pMut2 and pCure2-I47 (FIG. 5B, Table 2) were then transformed with the MccI47-producing plasmid (pMut2-I47) for in vivo applicability (FIG. 2B). pMut2-I47 contains the same genetic machinery for MccI47 production and export as pBBAD-I47 (FIG. 1A), however, it uses a proD-like (Davis J H et al., construction and characterization of a set of insulated bacterial promoters. Nucleic Acids Research. 2010; 39(3):1131-41) insulated strong constitutive promoter (J23119) for mciA mciI expression for maximum microcin output in vivo. Further, the pMut2 backbone allows plasmid retention without selection pressure. Since wildtype EcN harbors genes for the microcins MccM (mcmIA) and MccH47 (mchIB), we generated an EcN MccH47/MccM knockout strain (EcN^{ΔH ΔM}) to exclude potential interference by these microcins in the downstream experiments (FIG. 6, FIG. 7). Therefore, the following strains were used in the remaining set of experiments: (i) EcN, (ii) EcN^{ΔH ΔM}, and (iii) EcN^{ΔH ΔM}-I47, with the latter harboring pMut2-I47.

Since pMut2-I47 is 2.6-times larger than the native pMut2 (5514 bp), we evaluated pMut2-I47's retention in a serial passage experiment without antibiotic selection for ten consecutive days and approximately 200 generations in total. We did not observe a reduction in colony forming units (CFUs) carrying the plasmid (linear mixed effect modeling, p value>0.05; FIG. 2C) but intriguingly record a higher plasmid copy number for EcN^{ΔH ΔM}-I47 (mean±SD, 17.4±1.5) compared to EcN (12.9±0.8) as estimated by quantitative PCR (linear mixed effect modeling, p value<0.001; FIG. 2D), possibly caused by MccI47 accumulation during the growth phase. This suggests that even though we increased the plasmid size 2.6-fold, pMut2-I47 is retained stably long-term without the need of selection. We then confirmed EcN^{ΔH ΔM}-I47 killing activity using static inhibition assays. EcN^{ΔH ΔM}-I47 produced a zone of inhibition of more than 2.3 cm in diameter against CR K. pneumoniae (BAA 1705) irrespective of the addition of ampicillin for antibiotic plasmid retention (FIG. 2E). Simultaneously, the two respective controls EcN (1) and EcN^{ΔH ΔM} (2) were unable to produce any halo of inhibition. Taken together, these data provide the evidence for a novel EcN strain engineered to constitutively produce MccI47 from a self-retaining plasmid in vitro, which is suitable for application in a murine model to challenge CR K. pneumoniae colonization.

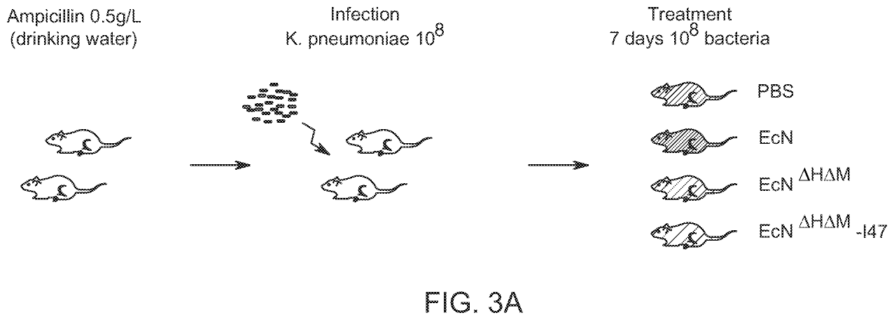

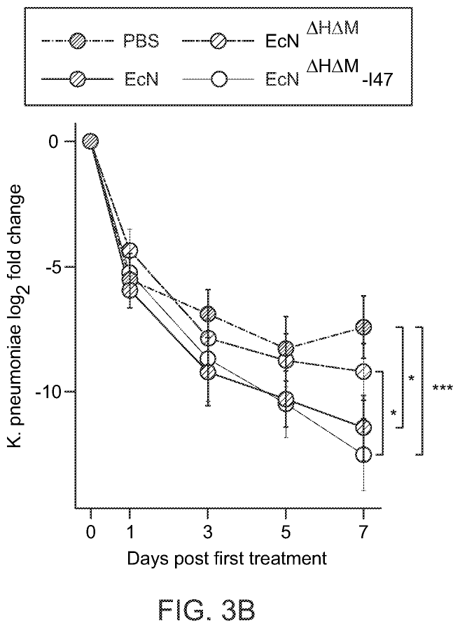

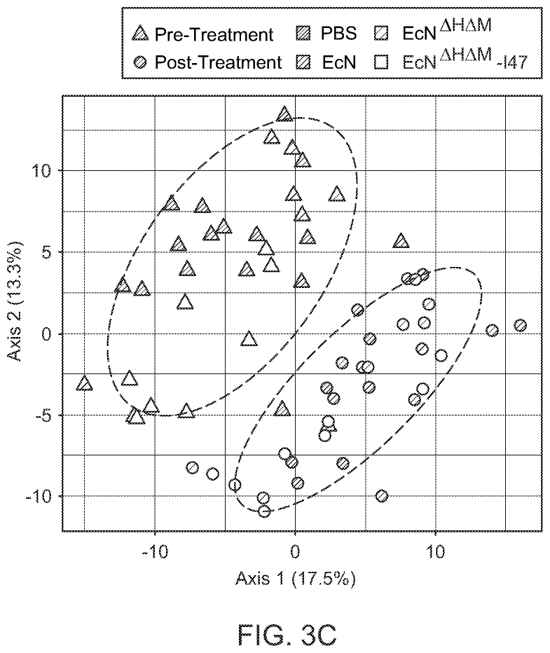

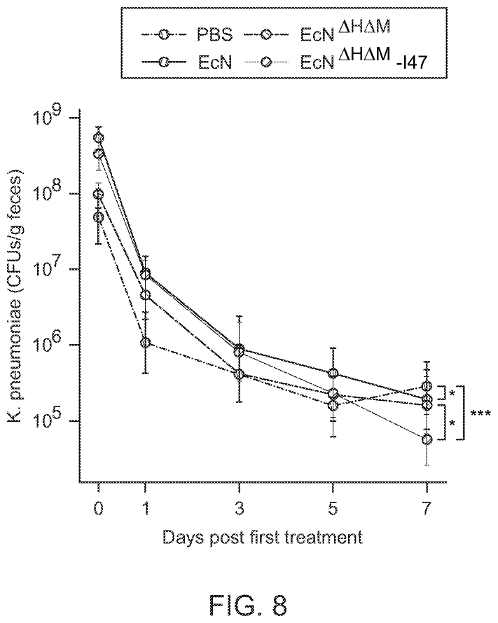

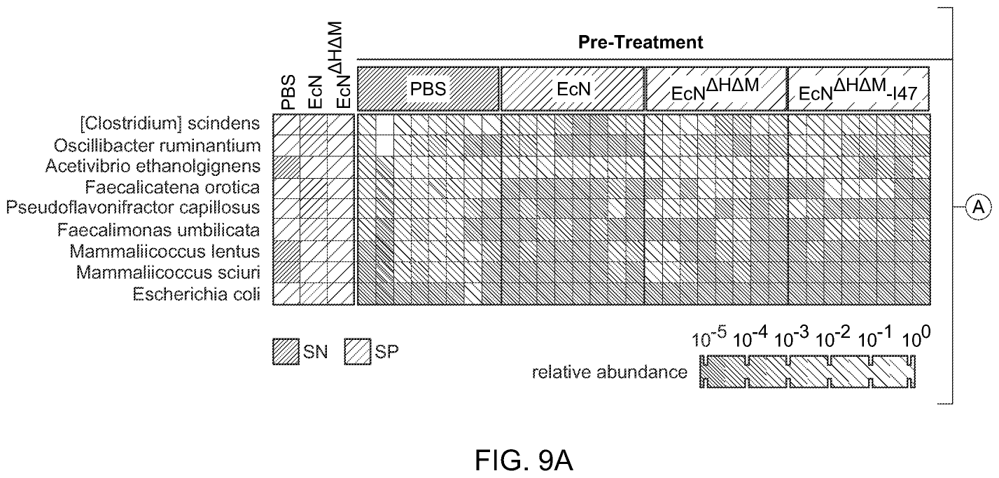

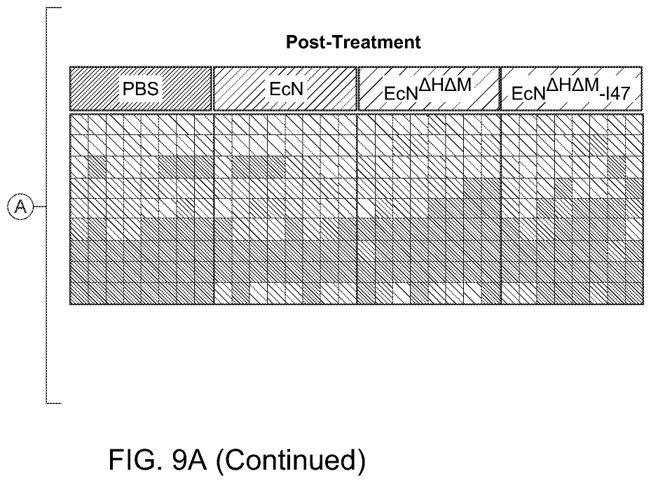

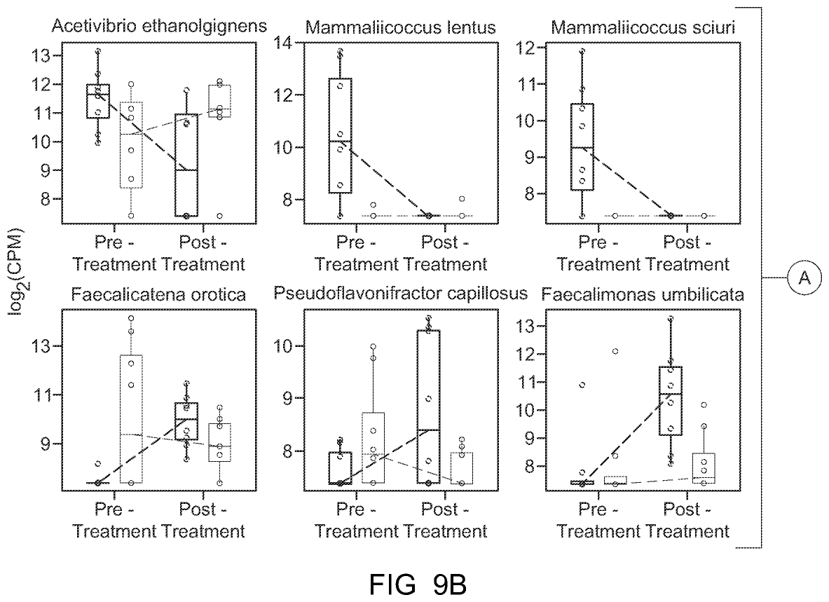

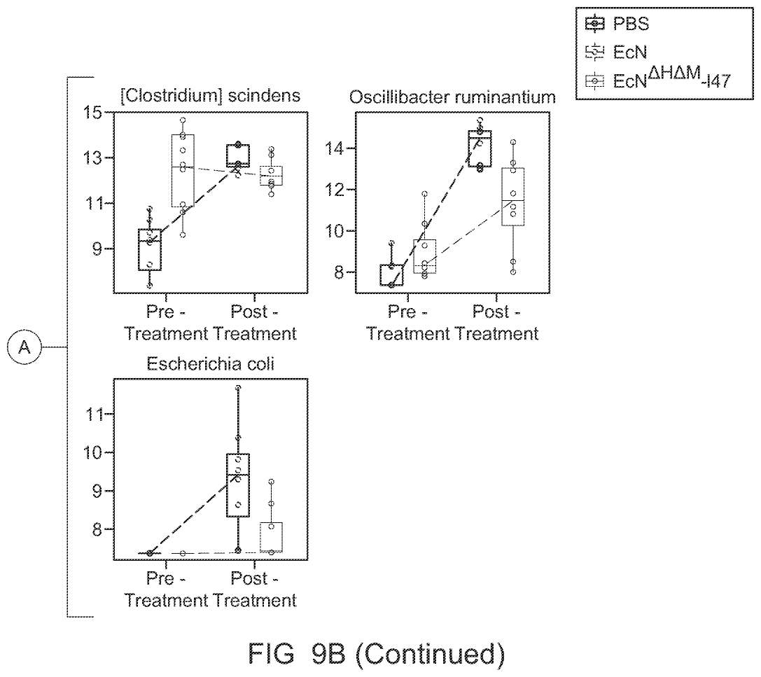

We then evaluated MccI47's ability to reduce gastrointestinal carriage of CR K. pneumoniae by administration of EcN^{ΔH ΔM}-I47 in vivo and performed a decolonization experiment in a specific-pathogen-free mouse model, as described in previous references (Young™ et al., Animal Model To Study Klebsiella pneumoniae Gastrointestinal Colonization and Host-to-Host Transmission. Infect Immun. 2020; 88(11):e00071-20; Buffie C G et al. Precision microbiome reconstitution restores bile acid mediated resistance to Clostridium difficile. Nature. 2015; 517(7533):205-8; and Caballero S et al. Cooperating Commensals Restore Colonization Resistance to Vancomycin-Resistant Enterococcus faecium. Cell Host Microbe. 2017; 21(5):592-602.e4). Briefly, we treated C57BL/6J mice with ampicillin for seven days to disrupt the resident microbiome, before challenging them with 10⁸ cells of CR K. pneumoniae (BAA 1705), for which we had demonstrated MccI47-mediated inhibitory effects in FIGS. 1B-1C, Table 1, and FIG. 2D. One week after CR K. pneumoniae application, each mouse was orally administered PBS or 10⁸ cells of either EcN, EcN^{ΔH ΔM} or EcN^{ΔH ΔM}-I47 daily for seven days (FIG. 3A). Fecal samples were collected throughout the experiment and plated to quantify absolute abundances of CR K. pneumoniae. The decolonization efficacy was tested for differences between the treatment groups by running linear mixed effect modeling as described in detail in the Methods section. Daily administration of EcN^{ΔH ΔM}-I47 causes a significantly higher decay in CR K. pneumoniae abundance (measured by fecal shedding) compared to treatment with PBS alone (p=0.0005) as well as treatment with the control strain EcN^{ΔH ΔM} (p=0.042) (FIG. 3B, FIG. 8). Moreover, treatment with the wildtype EcN also causes a significant decline in CR K. pneumoniae abundance compared to PBS (p=0.009) but not EcN^{ΔH ΔM} (p=0.26) or EcN^{ΔH ΔM}-I47 (p=0.37), supporting the previously described role of class IIb microcins in niche competition. Importantly, when compared to PBS, EcN^{ΔH ΔM}-I47 (time:treatment interaction coefficient=−0.22) exhibits a stronger decolonization effect than EcN (time:treatment interaction coefficient=−0.16). To determine whether the treatments resulted in alterations of the resident microbiota, we analyzed the relative bacterial abundances before and after treatment through 16S rRNA profiling. Principal coordinates analysis of pre and post treatment groups revealed significant community changes for all samples (PERMANOVA, p<0.0001) likely caused by the time passed since the antibiotic treatment and the recovery of the resident microbiota (FIG. 3C). However, we could not find any live biotherapeutic product-dependent changes in the community caused by the different EcN strains (PERMANOVA, p=0.81). Differential analysis using LimmaVoom resulted in only three significantly different bacterial species, when comparing EcN^{ΔH ΔM}-I47 with the PBS group (Acetivibrio ethanolgignens, Mammaliicoccus lentus, Mammaliicoccus sciuri), which may be due to the use of sensitive statistical testing, since none of them displays consistent signal over most samples across the treatment groups (FIGS. 9A-9B). Therefore, we can conclude that in contrast to commonly prescribed antibiotics, treatment with the engineered strain has minimal effects on the GI microbiota, with the addition that our engineered biotherapeutic strain EcN^{ΔH ΔM}-I47 selectively targets CR K. pneumoniae. Taken together these data demonstrate that the oral delivery of an engineered probiotic E. coli strain overproducing MccI47 while passing through the GI tract is a viable approach to reduce CR K. pneumoniae colonization in vivo.

### Methods

Bacterial strains and plasmids. This study used the Escherichia coli strains NEB100 (New England Biolabs, Ipswich, MA), Nissle 1917 as well as all strains listed in Table 1. Strains in Table 1 were purchased from ATCC (Manassas, VA). All plasmids in this study have been transformed into cells using electroporation with the Bio-Rad Micropulser™ (Bio-Rad Laboratories, Hercules, CA) and were created using Gibson Assembly (Gibson D G et al., Enzymatic assembly of DNA molecules up to several hundred kilobases. Nature Methods. 2009; 6(5):343-5) using the Gibson Assembly Master Mix (New England Biolabs, Ipswich, MA) and custom DNA oligonucleotides purchased from Integrated DNA Technologies (Coralville, IA). For pBBAD-H47 and pBBAD-I47, four fragments were amplified by PCR and assembled: (1) linearized pUC19, (2) araC and P_{BAD} from pTARA (Wycuff D R, Matthews K S. Generation of an AraC-araBAD promoter-regulated T7 expression system. Anal Biochem. 2000; 277(1):67-73) (Addgene #39491), (3) the microcin and immunity genes for MccH47 (mchXIB) or MccI47 (mciIA) originating from pEX2000 (Gaggero C et al., Genetic analysis of microcin H47 antibiotic system. J Bacteriol. 1993; 175(17):5420-7) as well as (4) the genes mchCDEFA originating from pPP2000. Plasmid pHMT-H47 has been described and used in Palmer J D et al., ACS Infectious Diseases. 2020; 6(4):672-9. For plasmid pHMT-I47 a total of seven fragments was assembled: (1) linearized pUC19, (2) chloramphenicol resistance cassette from pTARA (Addgene #39491), (3) lac and tac promoter from pMAL-c5X (New England Biolabs, Ipswich, MA), (4) MBP, amplified using primers to add a 6× Histidine N-terminal tag, from pMAL-c5X, (5) mciA from pEX2000, (6) mciI from pEX2000, and (7) mchCDEFA from pPP2000. To cure the native pMut2 plasmid from E. coli Nissle 1917, pCure2-I47 was assembled from four fragment: (1) linearized pMut2, (2) chloramphenicol resistance cassette from pTARA (Addgene #39491), (3) mciA from pEX2000, (4) lac and tac promoter from pMAL-c5X (New England Biolabs, Ipswich, MA). The modified replacement plasmid for pMut2, pMut2-I47, was created from four fragments: (1) linearized pMut2, (2) ampicillin resistance cassette from pUC19, (3) insulated promoter proD, where the promoter region was replaced with the strong constitutive promoter J23119 (BBa J23119), (4) mciIA to mchCDEFA from pBBAD-I47. All plasmid sequences and maps have been deposited as .dna files at the Gitlab website. Chromosomal modifications were obtained through lambda Red recombination using pKD46 and FLP-FRT recombination using pCP20 as described by Datsenko and Wanner (Datsenko K A, Wanner B L. One-step inactivation of chromosomal genes in Escherichia coli K-12 using PCR products. Proceedings of the National Academy of Sciences. 2000; 97(12):6640-5). Briefly, a kanamycin resistance cassette flanked by flippase recognition target (FRT) sites was amplified by PCR from pKD4 and then transformed into the respective electrocompetent EcN strain harboring plasmid pKD46. For strain EcN^{ΔH ΔM} the kanamycin resistance was removed after the knockout of mchIB by inducing the flippase from pKD20, before it was reintroduced for the mcmIA knockout. All modifications were confirmed using Sanger sequencing (FIG. 6).

Plasmid curing and assessment of retention. To cure the native plasmid pMut2 from E. coli Nissle 1917, the inducible plasmid pCure2-I47 was created for counterselection. After transformation of pCure2-I47 into E. coli Nissle 1917, plasmid presence was confirmed by selective plating on chloramphenicol (25 μg/ml) and PCR. Positive clones were individually grown over night at 37° C. under strong antibiotic selection (500 μg/ml) in liquid LB medium to skew plasmid competition towards pCure2-I47 and to displace pMut2. Resulting cultures were plated on LB agar and the loss of pMut2 was confirmed by PCR (Table 2).

TABLE 2
Primers used for plasmid verification in FIG. 5B
and quantitative PCR in FIG. 2D

| | | | Fragment |
|---|---|---|---|
| Primer name | Sequence | Target | size |
| pMut1_MobA_F | GTGCCCTGTTATCCAGGCTTATGG | pMut1 | 1208 bp |
| pMut1_ORF4_R | AGGTTGAAGGTCTCAGAGAATGAGAC | | |
| pMut2_ORF2_F | ATGTTAATCTGCTATTTGAATAGTCGAGTACGC | pMut2 | 1296 bp |
| pMut2_R | GCTCGTCATCGATCCGAATATTAATCG | | |
| pMut2_ORF2_F | ATGTTAATCTGCTATTTGAATAGTCGAGTACGC | pCure2-I47 | 1493 bp |
| pCure2_lacI_R | CGACATCGTATAACGTTACTGGTTTCAC | | |
| pMut2_ORF2_F | ATGTTAATCTGCTATTTGAATAGTCGAGTACGC | pMut2-I47 | 244 bp |
| pMut2_AmpR_R | TCTACACGACGGGGAGTCAGG | | |
| qPCR_gyrB_F | CATGGAGCGTCGTTATCCGA | gyrB | 147 bp |
| qPCR_qyrB_R | CTGCCGTGCTGTTCTTTGTC | | |
| qPCR_pMut2_F | CAAAGCCCCGAAATCATGCTC | PmUT2 | 186 bp |
| qPCR_pMut2_R | CGGAGAAGTACGGCTTGTGG | | |

Resulting clones were again individually grown over night at 37° C. in LB with the addition of 0.5 mM IPTG. Since pCure2-I47 expresses the MccI47 toxin (mciA) without the immunity peptide (mciI) under IPTG induction, cells harboring pCure2-I47 are killed in response to the addition of IPTG and therefore forced to lose the plasmid to survive. Resulting cultures were plated on LB agar and loss of pMut2 and pCure2-I47 was confirmed by PCR (FIG. 5B). Subsequently, pMut2-I47 was transformed into cured cells and retention without antibiotic selection was assessed by a serial passage experiment. Five independent replicates of EcN^{ΔH ΔM}-I47 were initially grown under ampicillin selection (100 μg/ml) overnight at 37° C. in liquid LB medium (to day 0) and then subcultured 10⁻⁶ daily for ten days (until day 10), allowing for the growth of approximately 20 bacterial generations per passage. Each biological replicate was plated daily on LB agar using three technical replicates with and without ampicillin (100 μg/ml) to quantify the percentage of CFUs retaining the plasmid over time. To test for the effect of time on the number of CFUs of EcN^{ΔH ΔM}-I47 retaining pMut2-I47 we ran linear mixed effect modeling in ‘R’ using the lmer from the lmerTest package. Specifically, we fit models predicting the percentage of CFUs having the pMut2-I47 plasmid as a function of time and using replicate ID as random effect. To assess the average plasmid's copy number per bacterium, cells contained in 1 ml from each daily passed culture were harvested and frozen at −80° C. until qPCR. To compare pMut2-I47 copy number with the copy number of the native pMut2, EcN was also subjected to the same serial passage assay and related procedures as control. Plasmid copy numbers have been assessed using qPCR by comparing the signal from the plasmid pMut2 to that from the chromosomal gene DNA gyrase subunit B (Table 2) (Lee C et al., Absolute and relative QPCR quantification of plasmid copy number in Escherichia coli. J Biotechnol. 2006; 123(3):273-80). The frozen bacterial pellet was resuspended in 1 ml of sterile PBS, incubated for 5 min at 95° C. and used as template in the qPCR reaction in a final 10⁻⁴ dilution with the iTaq Universal SYBR Green Supermix (Bio-Rad Laboratories, Hercules, CA). The qPCR was run in a StepOnePlus Real-Time PCR System (Applied Biosystems, Waltham, MA) and plasmid copy number was calculated using the ΔΔCt, assuming an efficiency of 100%. To test for differences in the number of copies of pMut2-I47 (carried by EcN^{ΔH ΔM}-I47) versus the number of copies of pMut2 (carried by EcN) and the effect of time, we ran linear mixed effect modeling in ‘R’ using the lmer from the lmerTest package. Here we fit models where the copy number was predicted as a function of the strain (i.e., EcN^{ΔH ΔM}-I47 vs. EcN), time and the interaction of the two and using replicate ID as random effect.

MccI47 purification. The MBP-MccI47 fusion protein was expressed from pHMT-I47 and purified as previously described. Briefly, overnight cultures of the E. coli NEB100 strain harboring pHMT-I47 were grown in LB under antibiotic selection (100 μg/ml ampicillin, 25 μg/ml chloramphenicol). The culture was diluted 1:50 in prewarmed LB medium and incubated at 200 rpm and 37° C. After 90 min 2,2-dipyridyl was added to a final concentration of 0.2 mM. After two more hours (approximate optical density at 600 nm OD₆₀₀=0.2), MccI47 production was induced with 0.5 mM IPTG for five hours. The cells were then harvested by centrifugation, resuspended in column buffer (200 mM NaCl, 20 mM Tris-HCl, pH 7.5) and frozen until purification at −20° C. The cells were slowly thawed in cold water, sonicated, and centrifuged at 4° C. with 15,000 xg for 10 min to remove the debris. The lysate was then passed through an amylose resin (New England Biolabs, Ipswich, MA) column and finally the MBP-MccI47 fusion proteins were eluted into 30 ml using 10 mM maltose in the column buffer. The MBP-MccI47-containing solution was concentrated using MilliporeSigma (Burlington, MA) MWCO 10,000 filters and then digested with 10 μl Tobacco etch virus nuclear-inclusion-a endopeptidase (TEV) (New England Biolabs, Ipswich, MA) overnight at 4° C. The next day the solution was brought to room temperature, another 5 μl TEV were added, and the digestion was incubated for additional two hours, resulting in free MccI47 in the solution. The histidine-tagged TEV and MBP were extracted from the solution using a Ni-NTA agarose resin (Qiagen, Hilden, Germany) as previously described.

Plate inhibition assays. Solid media inhibition assays were performed as described previously. Briefly, individual bacterial colonies of the microcin-producing strains (MccH47 or MccI47) were picked with a sterile pipet tip and stabbed into the solid LB agar. Iron-limited conditions during the growth phase were created by supplementing the media with 0.2 mM 2,2-dipyridyl. For the inducible constructs pBBAD-MccH47 and pBBAD-MccI47 the media was supplemented with 100 μg/mL ampicillin for plasmid retention and 0.4% L-arabinose for microcin production. For the self-retaining plasmid pMut2-MccI47 no inducing agent was necessary. Here, were indicated, ampicillin was locally added to the media by drying a drop of sterile water containing 500 μg ampicillin before adding the colony. All plates were incubated for 48 h and the bacteria were inactivated with chloroform vapors and 10 min of ultraviolet light. For the overlay, target bacteria were diluted from an overnight culture (E. coli BAA 196-1:200; K. pneumoniae BAA 1705-1:1000) in 3 ml LB with 0.2 mM 2,2-dipyridyl and 100 μg/mL ampicillin. Then molten agar was added to the media to a final concentration of 0.75%. 3.5 ml of the soft agar medium was then evenly spread on the plate, cooled and the plate was incubated for 16 h at 37° C. before imaging.

Minimum inhibitory concentration (MIC) assays. The MIC assays were carried out in sterile 96-well round bottom microplates (CELLTREAT Scientific Products, Pepperell, MA) and were set up with the following media. The first row was filled with 20 μl 2× LB with 0.4 mM 2,2′-dipyridyl and 20 μl of purified MccI47 in amylose resin elution buffer (200 mM NaCl, 20 mM Tris-HCl, 10 mM maltose, pH 7.5), resulting in the highest MccI47 concentration for the assay in 1× LB, 0.2 mM 2,2′-dipyridyl, 0.5× amylose resin elution buffer. All other wells were loaded with 20 μl 1× LB, 0.2 mM 2,2′-dipyridyl, 0.5× amylose resin elution buffer and two-fold serial dilution were performed across the plate. Target bacteria were grown over night in standard LB at 200 rpm at 37° C., and added to a final dilution of 104 into the individual wells. The plates were incubated in the dark at room temperature with gentle agitation and MICs were determined as the lowest concentration for which no growth was observed after 24 h. All reported MIC values represent the median of at least three biological replicates from independent MccI47 purifications.

Animal study. The mouse experiment was carried out under an institutionally approved Institutional Animal Care and Use Committee (IACUC) protocol. Per arm 16 C57BL/6J mice (four male, twelve female) at seven weeks of age were housed in same-sex pairs under specific pathogen-free conditions. Each mouse was individually marked, so they could be traced throughout the entire experiment. To achieve robust gastrointestinal CR Klebsiella pneumoniae (strain BAA 1705) engraftment, all mice were treated with 0.5 g/l ampicillin in the drinking water for seven days. We confirmed that CR K. pneumoniae (strain BAA 1705) was not already present in the mice by selective plating of undiluted feces suspension at both time of arrival as well as on the morning right before infection. For infection, mice were deprived of food and water for four hours and then given 10⁸ cells of CR K. pneumoniae (strain BAA 1705) in 20 μl phosphate buffered saline (PBS) by pipet. After one week of engraftment, the four arms were given daily for seven days either (i) PBS, or 10⁸ cells days of either (ii) EcN, (iii) EcN with a knockout of the genes mchIB (MccH47) and mcmIA (MccM) to prevent interference of the other class IIb microcins in the EcN genome (EcN^{ΔH ΔM}), or (iv) EcN with the same gene knockouts and constitutive MccI47-production from a modified version of the native plasmid pMut2 (EcN^{ΔH ΔM}-I47). For administration the mice were again deprived of food and water for four hours, then given PBS alone or the respective cells orally by pipet in 20 μl PBS, and transferred to a clean cage after the first treatment. Fecal samples were taken before the first administration (day 0), day 1, 13, day 5, and day 7 after the first treatment to assess the colonization of CR K. pneumoniae (strain BAA 1705). For sampling, mice were placed into sterile isolation containers and at least two fecal samples (approximately 0.03 g each) were collected into sterile two 1.5 ml microcentrifuge tubes. One sample was snapfrozen and stored at −80° C. until DNA extraction, the other one was weighed, resuspended in sterile PBS and kept on ice until plating. To determine the bacterial shedding in CFU/g feces, the samples were shortly spun in a minicentrifuge to pellet larger particles and plated in 10-fold serial dilutions from the supernatant on respective antibiotic plates and incubated at 37° C. for 16 h, which resulted in a detection limit at 10² CFU/g feces. To compare the effect of treatment on the CR K. pneumoniae reduction rate, we ran linear mixed effect modeling of the form, log₁₀(CFU counts)˜Time (Days)+Treatment+Time:Treatment+1|MouseID, using MouseID as random effect. The model was fitted to the data using the lmer function from the lmerTest (Kuznetsova A, Brockhoff P B, Christensen R H B. lmerTest Package: Tests in Linear Mixed Effects Models. Journal of Statistical Software. 2017; 82(13):1-26) package in ‘R’. The interaction term “Time:Treatment” was inspected to determine significant differences in CR K. pneumoniae reduction rate across treatments. Significance and magnitude of interaction coefficients were inspected to determine differences among the treatment type. Data and ‘R’ code to perform statistics have been deposited at the Gitlab website.

Relative bacterial abundance analyses. DNA was extracted from frozen fecal pellets with the DNeasy Powersoil Pro Kit by Qiagen (Hilden, Germany) according to the manufacturer's protocol. The bacterial 16S rRNA gene (variable regions V3 to V4) was subjected to PCR amplification using the using the universal 341F and 806R barcoded primers for Illumina sequencing. Using the SequalPrep Normalization kit, the products were pooled into sequencing libraries in equimolar amounts and sequenced on the Illumina MiSeq platform using v3 chemistry for 2×300 bp reads. The forward and reverse amplicon sequencing reads were dereplicated and sequences were inferred using dada2 (Callahan B J et al, DADA2: High-resolution sample inference from Illumina amplicon data. Nature Methods. 2016; 13(7):581-3). To test for treatment specific differences compared to EcN^{ΔH ΔM}-I47, for every species, we ran linear mixed modeling of the form, Species (normalized counts)-Time (Day 0|Day 7)+Treatment+Time:Treatment+1|MouseID with MouseID as random effect using Limma/voom as in Wipperman M F et al. Gastrointestinal microbiota composition predicts peripheral inflammatory state during treatment of human tuberculosis. Nat Commun. 2021; 12(1):1141. Species were determined to be significantly different (Benjamini Hochberg adjusted p-value<0.05) for the interaction term “Time:Treatment” from the model. All ‘R’ code has been deposited at the Gitlab website. Raw microbiome data have been deposited on the European Sequencing Archive (ENA) accession number PRJEB48537.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically engineered microorganism, wherein the microorganism comprises
a microcin operon, and
a promoter for the microcin operon, preferably wherein the promoter is a constitutive promoter,
wherein the microcin operon comprises one or more microcin genes, and wherein either or both of the microcin operon and the constitutive promoter are heterologous to the microorganism;
wherein the microcin operon and the promoter are in a self-retaining vector.

2. The genetically engineered microorganism of claim 1, wherein the genetically engineered microorganism is a bacterium.

3. The genetically engineered microorganism of claim 1, wherein the genetically engineered microorganism is Escherichia coli.

4. The genetically engineered microorganism of claim 3, wherein the E. coli is E. coli Nissle 1917 (EcN).

5. The genetically engineered microorganism of any one of claims 1-4, wherein the self-retaining vector comprises one or more genetic modifications of a native vector of the microorganism.

6. The genetically engineered microorganism of claim 5, wherein the microorganism comprises a deletion of the native vector and the introduced self-retaining, genetically-modified vector.

7. The genetically engineered microorganism of any one of claims 1-6, wherein the self-retaining vector is a multicopy vector.

8. The genetically engineered microorganism of any one of claims 1-7, wherein the genetically engineered microorganism retains the self-retaining vector without antibiotic selection.

9. The genetically engineered microorganism of any one of claims 1-8, wherein the self-retaining vector is a pMUT1 or pMUT2 vector.

10. The genetically engineered microorganism of any one of claims 1-9, wherein the microcin operon comprises one or more Microcin H47 (MccH47) genes.

11. The genetically engineered microorganism of claim 10, wherein the microcin operon comprises one or more of mchB, mchC, mchD, mchE, mchF, mchA, mchX and mchI.

12. The genetically engineered microorganism of claim 10 or 11, wherein the microorganism comprises mchA.

13. The genetically engineered microorganism of any one of claims 1-9, wherein the microcins operon comprises one or more Microcin I47 (MccI47) genes.

14. The genetically engineered microorganism of claim 13, wherein the microcin operon comprises microcin genes mci, mciA, mchC, mchD, mchE, mchF and mchA.

15. The genetically engineered microorganism of any one of claims 1-14, wherein the microorganism comprises a deletion of the native Microcin H47 or Microcin M genes.

16. The genetically engineered microorganism of any one of claims 1-15, wherein the constitutive promoter is a J23119 promoter.

17. A self-retaining multicopy plasmid vector comprising:
a microcin operon comprising a set of microcin genes, and
a constitutive promoter, wherein the constitutive promoter allows for continual transcription and expression of at least one microcin gene.

18. The vector of claim 17, wherein the set of microcin genes comprises one or more Microcin H47 (MccH47) genes.

19. The vector of claim 18, wherein the set of microcin genes comprises one or more of mchA, mchB, mchC, mchD, mchE, mchF, mchX and mchI.

20. The vector of claim 17, wherein the microcins operon comprises one or more Microcin I47 (MccI47) genes.

21. The vector of claim 20, wherein the microcin operon comprises microcin genes mciI, mciA, mchC, mchD, mchE, mchF and mchA.

22. The vector of any one of claims 16 to 21, wherein the constitutive promoter is a J23119 promoter.

23. The vector of any one of claims 17 to 22, wherein the vector is a plasmid.

24. The vector of any one of claims 17-23, wherein the vector is a pMUT1 or pMUT2 plasmid.

25. The self-retaining multicopy plasmid vector of claim 17, comprising:
the microcin operon comprising mciI, mciA, mchC, mchD, mchE, and mchF;
a first constitutive promoter, wherein the first constitutive promoter allows for continual transcription and expression of the operon;
mchA, and
a second constitutive promoter, wherein the second constitutive promoter allows for continual transcription and expression of the mchA;

26. The self-retaining multicopy plasmid vector of claim 17, comprising:
the microcin operon comprising mchB, mchC, mchD, mchE, mchF mchX and mchI;
a first constitutive promoter, wherein the first constitutive promoter allows for continual transcription and expression of the operon;
mchA,
and a second constitutive promoter, wherein the second constitutive promoter allows for continual transcription and expression of the mchA.

27. The vector of claim 25 or 26, wherein the constitutive promoter is a J23119 promoter.

28. The vector of any one of claims 25-27, wherein the vector is a plasmid.

29. A composition for treating a bacterial infection, wherein the composition comprises the genetically engineered microorganism of any one of claims 1-16.

30. The composition of claim 29, wherein the composition is packaged in a capsule for intestinal delivery.

31. The composition of claim 29 or 30, wherein the bacterial infection is a gram-negative bacterial infection.

32. The composition of claim 31, wherein the bacterial infection is carbapenem-resistant Enterobacteriaceae infection, E. coli infection, Salmonella infection, and/or Shigella infection.

33. The composition of claim 32, wherein the carbapenem-resistant Enterobacteriaceae infection is a Klebsiella pneumoniae infection.

34. A method of treating intestinal dysbiosis, the method comprising:
identifying a subject as having intestinal dysbiosis; and
administering to the subject a therapeutically effective amount of a composition comprising the genetically engineered microorganism of any one of claims 1-16 or the composition of any one of claims 29-33.

35. The method of claim 34, wherein the subject is a human and the composition is administered by endoscopy, enteroscopy, colonoscopy, a nasoduodenal catheter, enema, or by oral administration.

36. The method of claim 35, wherein the composition is orally administered, optionally in a capsule.

37. A method of treating a bacterial infection, the method comprising:
identifying a subject as having a bacterial infection; and
administering to the subject a therapeutically effective amount of a composition comprising the genetically engineered microorganism of any one of claims 1-16 or the composition of any one of claims 29-33.

38. The method of claim 37, wherein the subject is a human and the composition is administered by endoscopy, enteroscopy, colonoscopy, a nasoduodenal catheter, enema, or by oral administration.

39. The method of claim 38, wherein the composition is orally administered, optionally in a capsule.

40. The composition of any one of claims 37-39, wherein the bacterial infection is a gram-negative bacterial infection.

41. The composition of claim 40, wherein the bacterial infection is carbapenem-resistant Enterobacteriaceae infection, E. coli infection, Salmonella infection, and/or Shigella infection.

42. The composition of claim 41, wherein the carbapenem-resistant Enterobacteriaceae infection is a Klebsiella pneumoniae infection.

43. A method of reducing a risk of a bacterial infection, the method comprising:
identifying a subject as having a risk of a bacterial infection; and
administering to the subject a composition comprising the genetically engineered microorganism of any one of claims 1-16 or the composition of any one of claims 29-33.

44. The method of claim 43, wherein the subject is being administered one or more antibiotics.


SEQUENCE LISTING (17 sequences)

SEQ ID NO: 1 - DNA, 1119 bp, Escherichia coli
  source 1..1119: mol_type = genomic DNA
atgcgaaaacgtattctttttattggcccaccgctgtacggtttgttatacccattgatttctctggctcaggcctttcgtgtaatcggacatgatgtagtaattagtagtgctggcaaattcgcgaataaagcagcagaagctggactggttgtttttgatgcagttccaggtttagattcagaggctggatatcgccatcaggaagagttgaggaaaaaaagtaatattattggtcatttctctttttttagcgatgaaatggcagataacctcatcgattttgcaggaaaatggaggccagatttaatagtctatcccccgcttggtccggcaggcccattggttgctgctaaatatagaattccttcagtgatgctggctgttggattcgcgcatacatctgcccatattcagatgttaaaccgttctttaagcaatgcttacaggcggcatggagtcagcggtccactatgtgatttagcatggattgatgttgctcccccaagtatgagcattcttaaaaatgctgaagaaccggttatctcaatgagatatattccttataacggaggtgctgtaaaggaaacatggtgggacagggattctgatcgaaaacgtttactcatcagccttggcactgtaaaaccaatggttgatggtctggagctgatttcatgggttatggattctgcaaatgaagttgatgctgatatcattttgcaacttgcaataaatgctcgtactggattacgaaaactaccatcaaatgtacgtctggttgactggatacctatgggtgtattccttaatggagctgatggatttattcatcatggtggcgcaggtaataccctgacagcgttgtatagtgggataccacagattgtgtttggcgaaggtgcagatcgctctgttaatgcagaaattgttgcgatgcgtgggtgtgggattattccggacaagcatggactgaccagtgatttggtaaatcgcctgctttatgatgattcactacgcttctgttcagatcaggtagccgctgaaatggctgaacaacccagtcctgcagagatcgcagaggttttgatgagaaaattaaaaaacaacgggaaataa

SEQ ID NO: 2 - DNA, 1551 bp, Escherichia coli
  source 1..1551: mol_type = genomic DNA
atgagtcatcagtgttcactttctgaactgaatgaaaacctggtgcctttcactgccaggcagatcaagtcctcattaatctggtgtgcagaggatgtcagaaatccaggcgagctgcaaaatgcctgcagttatattatcgatcctgacagtacggcttctgccaaagtgttccatgcagagcgctatggtggcagtggtattcagcgtaatggaggtggtgcacgttgtgggtttgatggtaactaccaggttaaaggaataggaagtaatccgttggttggtgaaggtactgacgaacgtcattctaatggtgcactcggcgctgttcatgcaatatatgaggctttgtggggagaagtactggctcaaatattaccttatagtgctgtgcgggttcgggcggttttacttacagatctctatactgaaaaggcatttgagcgctccggtatgaaatcacgaagagccctgttggtacgtgagcctgttgttcgcccggcgcattttgaacgggcaccatacttccaagtaaaaccggagtattccagtcagttaattcacgatgcctgtcgggttagatctgtgatccacaagctgccaggatatctacctgtaccaccggaagaaattgatgctgaagcacgaactgatccccggatttattgcattgagggattatgtgaactggcacgtcgtgaggcctggcaaatggcattttgtcgaacacgtttcctgagattgacaacttctccttctaatattgcaatggatggcagattaatggattttaacggactcagttgctcgtttccgggagattccccagctgattttgggtataaactaagattagctgaactggcaaaagaaccgatggtacttatgcaagggctgtctgatctctgcttgtatatcggaaaatatatgtttgaccctgacttcactcttgcagcccgtttgaaggttgaggagatatttcagaaaacttttcatgaagcatgttattactgttatctagaactgttgggtattcctggagaatttataacacaaaaagagatacctgatatattgaaacaactggttaacagttttgttgcattactcaataaatactgcgagaaatcacatgcccaagatattgtcaatcaggatggttcaccattgcaaaagttggttgtgacgctaatccatcataggcataatcaaaagcaggcactgaatagtagcatcaagaatgatgtttatttcaccgttgcacaacagtgtttttcccagactatccactggctgacgcaaggcagtaccagacgtcagataaatgcttcattactcctgaaagaaattgaacatcataccatgaaaaggctgcaacccagggaagagctgaggaaagagaatatgtgcgaaaaaattgccatcctgctggataatcatggcgatgatcccctttttttacaagaagcaatttctgatatgaaaaattttatgcttaagttttccagagatgcatttggatatcttgaaccgataagaaacacagtgtaa

SEQ ID NO: 3 - DNA, 453 bp, Escherichia coli
  source 1..453: mol_type = genomic DNA
atgtcttatataagggaaaccatcagaggaaaagatgaatggactgtttatgaacagataggttttgcggtcagttgtatgctctacaatcgtaattacagtctgtatccggtgttaaccattcaatactggactgaatatgcgatacagcataatcagattaaattcctgtttgattcacgaggttttccactggcgtatataacctgggcatatcttgaggctgatacggaagcgcgcctgctcagggatccagaattcaggttgcatccgtctgaatggaatgaagatggaaggatctggatcctggatttctgttgtaaaccaggctttggtcgaaaagttattgactatctcatacagcttcagccatggggggaaggagaagtacgatggttaagcaggcgaaagaaaattgtgacatacatccctgagcggctgcataaaacgtag

SEQ ID NO: 4 - DNA, 228 bp, Escherichia coli
  source 1..228: mol_type = genomic DNA
atgcgagaaataacagaatcacagttaagatatatttccggggcgggaggtgcgccagcgacttcagctaatgccgcaggtgctgcagctattgttggagctctcgccggaatacctggtggtccacttggggttgtagttggagccgtatctgccggtttgacaacagcaattggctcgaccgtgggaagtggtagtgccagttcttctgctggtggcggtagctaa

SEQ ID NO: 5 - DNA, 1275 bp, Escherichia coli
  source 1..1275: mol_type = genomic DNA
ttgtttcgtcaggatgctttagaaaacagaaaaatgaagtggcagggacgggcaatattacttcccggaataccactatggttaatcatgctgggaagcattgtgtttattacggcatttctgatgttcattattgttggtacctatagccgccgtgttaatgtcagtggtgaggtcacaacctggccaagagctgtcaatatatattcaggtgtacagggatttgttgtcaggcaatttgttcatgaagggcagttgataaaaaaaggggatcctgtttatctgattgacatcagtaaaagtacacgtagtggtattgtcactgataatcatcggcgggatatagaaaatcagctggttcgtgtggacaacattatttcccgtctggaagaaagtaaaaaaataacgttagataccctggaaaaacaacgtctgcaatacacagatgcgtttcgtcgctcatcagatattatacagcgtgcagaggaagggataaaaataatgaaaaacaatatggagaattacagaaactatcaggcaaaagggctgattaataaagatcagttaactaaccaggtggcattatattatcagcaacaaaacaatcttctcagcctgagcggacagaacgaacagaatgccctgcagataaccactctggagagtcagattcagactcaggctgcagattttgataaccgtatctaccagatggaactgcaacggtacgagttacagaaagaactggttaacactgatgtggagggcgaaattattatccgggcgttgactgacgggaaagttgactccctgagtgtcactgtcgggcaaatggtcaataccggagacagccttctgcaggttattcctgagaacattgaaaactattatcttattctctgggtcccaaatgatgctgttccttatatttcggctggtgacaaagtgaatattcgttatgaagcctttccggcagaaaaatttgggcagttctctgctacggttaaaactatatccaggactcctgcgtcaacacaggaaatgttgacctataagggtgcaccacagaatacgccgggcgcctctgttccctggtataaagtcattgcgatgcctgaaaagcagattatcagatatgacgaaaaatacctccctctggaaaatggaatgaaagccgaaagtacactatttctggaaaaaaggcgtatttaccagtggatgctttctcctttctatgacatgaaacacagtgcaacaggaccgctcaatgactaa

SEQ ID NO: 6 - DNA, 2097 bp, Escherichia coli
  source 1..2097: mol_type = genomic DNA
atgactaacgggagtttcagacaaattataaatcagcttgatatgcgctggcgacgtcgtgttccggttattcatcagacggagaccgctgaatgtggactggcctgcctggcaatgatatgcggtcattttggtaagaatattgacctgatatctcttcgccggaagtttaatctctcggcccgtggagcaaaccttgcaggaatcaatggaatagcggagcagctggggatggtcacccgggctctttcactggagctggatgaacttggtgccctcaaaatgccgtgtattctccactgggatttcagtcactttgtcgtgctggtcagcgtaaagcgtaaccgttatgtactgcatgatccggccagaggcagaagatatctcggtcgggaggaaatgagccggtattttacgggcattgcacttgaggtctggcctggaagtgaattcctggcggaaacccagcagatccgcataagtctccgttcactgattaacagtatttacggtattaaaagaacactggcgaaaattttctgtctgtcagttgtaattgaagcaatcaatctggtaatgccggtggggactcagctggttatggatcatgcgattccggcgggggacagagggctgctgacgcttatttctgctggcctgatgttctttatattgctcagggccgcggtgagtatgctgcgtgcatggtcctcactggttatgagcacgctcatcaatatacagtggcagtcgggtctgtttaaccatcttctcagactgccgctggcgttttttgaacgccgtaaattaggtgatatccagtcgcgttttggctcccttgacactttgagggccacctttaccacctgtgtggttggggcaatcatggacagtattatggttgtgggggtttttgtgatgatgctgttatatggaggatatcttacctggatagtgctcggttttaccatggtttacgttcttattcgtctggtgacatacggctattaccggcaaatatcggaagaaactcttgtcaggggggcccgggccagctcctattttatggaaagcctgtatggtattgccacggtaaaaatccaaggtatggctgggatccggggaacacactggcttaacctgaaaatagatgcgatcaattcaggtattaagttaaccaagatggatttgctcttcggggggataaatacttttgttgccgcctgtgatcaggtggcgattttatggctgggtgcaagccttgtgatcgataatcagatgacaatagggatgtttgtggcatttggttcttttcgtgggcagttttcggatcgggttgcttcgctgaccagttttcttcttcaactgagaataatgagtctgcataatgagcgcattgcagatattgcactacatgaaaaggaagaaaagaaaccggaaattgaaatcgttgctgacatgagcccggtttcactggaaaccactgatttaagctaccggtatgacagccagtcagcacaggtattcagtggtctgaatttgtctgtggctccgggagaaagtgtggctataactggtgcctccggtgccggaaaaaccacattaatgaaagtattatgtggactgtttgaaccagatagtggaaaagtactggttaatggcacggatatacgtcaacttggaataaataattatcaccgtatgatagcctgtgttatgcaggacgaccggctattttcaggatcaattcgtgaaaatatctgtgggtttgcagaagaaacagacgacgaatggatgacagaatgtgccagagcaagtcatattcatgatgtgataatgaaaatgccaatggggtatgaaacgttaataggtgaactgggggaaggtctttccggcggtcaaaaacagcgtatattcattgcccgagctttataccggaaacctggaatattatttatggatgaggctacaagttctcttgatacagaaagtgaacgtttcgtgaatgctgccataaaaaaaatgaatatcacccgggtgattattgcacacagagaaactacgttgagaactgttgacaggattatttctatttaa

SEQ ID NO: 7 - DNA, 210 bp, Escherichia coli
  source 1..210: mol_type = genomic DNA
atgagttataaaaaactgtaccaattgacggctatatttagtttacctcttactatcttattggtttcactttcatcccttcggattgttggcgaagggaattcttatgttgacgtttttctaagctttataatatttcttggttttattgagctgattcatgggattcgaaagattttggtctggtcaggctggaaaaacggaagttaa

SEQ ID NO: 8 - DNA, 120 bp, Escherichia coli
  source 1..120: mol_type = genomic DNA
atggaatttgctacaaacagggttactgtaaatgacagtcggtcagcactgtcatcaactttgctgttgtctttgatcatgagcgccactctactggaatattctttatcgatgacctga

SEQ ID NO: 9 - DNA, 1266 bp, Escherichia coli
  source 1..1266: mol_type = genomic DNA
atgaaaaactatcttttccagactcccgaagatatttgtgtacagttaaaaaaaatgacacatcctgtcacaataagaacaacagatattgctaatttctggcactatcttgagtcagcaactcttccggtgatcacaaaaagcaccactacagaaaatcgggaggttacatttctgtggcgctcagagaaagcagtgcaaggcgtatatcttcgcctgaatcgtgttacagataaaaaagatgtcaaaaaaggactaatgactcatatcccttcgacagatatctggatgctgacactggtgttaccagcttcatatcggggctcatactcatttatagaaattcccacagatatgacacaaaaagacatatttcaactaggaagtcgcttctctccattacccggtaaatctgatccatttaacaaaacagcagaaataaatatacgaggattcggagaatcagtcctttctcttgatatggctcctgaacaaaaggaatgggatgatacttcccataaatgtacaggtattctttcaacattacattcctttgttgcaggatatcaacgccggattcgtttatattttccccagaatccaacatcagtacctcttggattacttgtgttacctgatgctgaaatatggtttgaccggatggatattacccgggcattagatatggccattaccactggtcatattgcgccaatggcaattatggggatagacaatattaatgaatctgatcgtatgaatatactgggaggcaataaagaacttatctttgatatagcggaaaatctgataccccagttatacagagactacccgaatatcgtatgggctggtcgttctaatactatactggccggtcagagcctcggtggagtgacagcactgatggcagctatatatgcgtcgacaacatttggtacaatcattagccactcaccttcaatgtggtggaaccctgaccagggcagcccgattttgtttactgagaatgatatctcctgggtaagtgagcagatactttcagcgcctccgaaagatgtaaatatccaacttggagtcggttctttagaaggtacaaccgtctcacatgttcagcggttgcatcagtcgttaatcgcagcaggtttggaaagtaacctcactgtctatgccggtggtcatgattatgcctggtggcgcggagcaattattgatgcattagcaaattataattgcaggaagatatcagataataactttgtgtaa

SEQ ID NO: 10 - DNA, 246 bp, Escherichia coli
  source 1..246: mol_type = genomic DNA
atgaattgtgataataatcacagaaatgaagaattcattgttacctttgataaaggcaacaagcaagacaattcaagacgaaaacacgataattttcctatagaggtagaatcctccgtagagctggagacacactgtatcacaaataataagtcggcttccggtatagtaacacatgactatgatgccgattatatttgtggttgtggtgaaattatgtgtcctggttgcggtcatgacctataa

SEQ ID NO: 11 - DNA, 234 bp, Escherichia coli
  source 1..234: mol_type = genomic DNA
atgagagaaatatcagataacatgcttgattccgtgaaaggagggatgaatcttaatggattacctgcttctactaatgtaatagatctacgtggaaaagatatgggaacatatattgatgctaatggagcatgctgggctccggatactccatccatcatcatgtatccggggggaagtggaccttcttatagtatgagtagttccacatccagtgcaaacagcggcagttaa

SEQ ID NO: 12 - DNA, 435 bp, Escherichia coli
  source 1..435: mol_type = genomic DNA
atgtatcttacgaaaaagattataataagtatgatgtttatattaccatctgctgcattttcatcagatccacctccccttcaacaatcgttagaaaaaacaacctatttttctataggtatgaatgggtttataggctatcagagcgaaggggaaaaattatacacacacattcttacattagataatcccgaagagatatttaaaaatataataaaaaatagaaagtcaactaaggagtctaaaatttatgctgcttgtgggctatattatttaaacgtagaaaatatagagtcattgtttaatgaaaatgataaacaagaatatgtgtctgtcttaagaggggatattttaacaaaaataaaactgaatgatattctgaattctgtgataataaatggttgcaacaccaaattaatatctgaacataaatga

SEQ ID NO: 13 - DNA, 34 bp, Synthetic construct
  source 1..34: mol_type = other DNA
ttgacagctagctcagtcctaggtataatgctag

SEQ ID NO: 14 - DNA, 156 bp, Synthetic construct
  source 1..156: mol_type = other DNA
acagctaacaccacgtcgtccctatctgctgccctaggtctatgagtggttgctggataacttgacagctagctcagtcctaggtataatgctagcagggagaccacaacggtttccctctacaaataattttgtttaactttagaggaggtaata

SEQ ID NO: 15 - DNA, 14486 bp, Synthetic construct
  source 1..14486: mol_type = other DNA
ttttgatctctttggtgcgcttttccatgagaatccgctgagaaagtttcgcacccaaagtgcgaattttcgcagtggatgcaaggggtttcggggggcggcgagccccctgaaacagtcacagacggcacctcgaagaggggacgctgtgtgtactggcttagtacagcatctatcgtacatcgaggtcgcatcacgcacaaacaaaaagccccgcaaaagcagggctgttatctgatataggttgttttgtctcacacggcagcggaagaggaatccgaagtggtactggtagtagtattggatgctgctgacgatattttccgctttgacccaaggcttaaataatcaatgcctgtaatcaacgatctcaatacgccttcggataccatagcgataaacgtatcttgctggttatggcttgcgatgcaaatcgtagcatcaccttttttatactttaaaacacctgctaaatatccattttcatctagaacactcttaagatgttcatttgttattgtttgtagcgtttgctttgtttcgcttcgagcatacgccttagctagcttccgagaaaaagcatccgcatcatgactatctttatttactcgctcaataaatttgcttaagtcaacaaatcccttaaaacgagtggacatattgttaacaaaatcagtggcagcattttttatccatgctttatagccaaaaaaacgctcgaaaacattttggtcgtagataaataccgtatcgccagcaaaaacaagagatgccttaccatcaatagaaatcatatcttgatctactcgagatagttcttttttgctaaacccataaaaattatttttctttcttgataagttttgaactggatattgcttcttgtatatggttatacaattgtcgtcattattcttactcaaaacgaaaatgattgagtcaacttttgatattagatccacactgtcaaaatcaacaattgggatattttcattgccatcaccaccaaacccagcatcaaacacaatgccagtcagaaacttcatttgctcattatcatactgctcagcatcatttaaaaatgaaatggtgttggtgcggtcacttagtgtattaacatctgatactatcacaacccgattttctaaatcagatatagttttctttatcacattatcaataatggactgttttagctcactgtcatttttaaggatggcaattttatagctaaaagagtccttagcacccgctttacctttatttttaaagttaaagtaagtgtgcaatgtaacatcgttaatatcacaatcaaaatgcttatacagttctaaaagctcttgtgctttttcttcattatgctccaaagcatcaagatctaaggcatcgtcactcatcatcattcctctatgatttttttcgcgaacgttaaataatcatcatgatttataactctgataaaatcattttcttttattaaatctttagataaaactatcaactcaccgtcttgcgttttttcccttccattagctaccacactgtaagtaatcttataggcagaaacattaaataatgacaatgttgggttgcagtgaattctttttgttttgatgtgcaaaaaaccgacgataatcaaaacaaacaaaaaattaactatatttgatggtttgcttaaatcagtaaagaccaacggcattatgtacgttgataaaaaagaaagatactcaccggattcttttttacatgaaacgacctttaactttcttgacaccgcaccggagtctaagtttttcaaaacccatcgataccaaatgtatgtataagaacaagttaaaatcaaagccccgcagatcactgacctcaatacagaaaatgttaatctgctatttgaatagtcgagtacgcattgaaattttccatccgcgccaggtcatgagattatcaaaaaggatcttcaccaagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacagctaacaccacgtcgtccctatctgctgccctaggtctatgagtggttgctggataacttgacagctagctcagtcctaggtataatgctagcagggagaccacaacggtttccctctacaaataattttgtttaactttagaggaggtaataatgtatcttacgaaaaagattataataagtatgatgtttatattaccatctgctgcattttcatcagatccacctccccttcaacaatcgttagaaaaaacaacctatttttctataggtatgaatgggtttataggctatcagagcgaaggggaaaaattatacacacacattcttacattagataatcccgaagagatatttaaaaatataataaaaaatagaaagtcaactaaggagtctaaaatttatgctgcttgtgggctatattatttaaacgtagaaaatatagagtcattgtttaatgaaaatgataaacaagaatatgtgtctgtcttaagaggggatattttaacaaaaataaaactgaatgatattctgaattctgtgataataaatggttgcaacaccaaattaatatctgaacataaatgagcaggaggtttttaaaatgagagaaatatcagataacatgcttgattccgtgaaaggagggatgaatcttaatggattacctgcttctactaatgtaatagatctacgtggaaaagatatgggaacatatattgatgctaatggagcatgctgggctccggatactccatccatcatcatgtatccggggggaagtggaccttcttatagtatgagtagttccacatccagtgcaaacagcggcagttaaataaataccatccggctatgccggcaggcactttttcgatagtgcctgtaaaatatatccctttcgtttaaagcagatagaagatccatttataagtgtgtttttatgttcaacactttactgatgaagatatgatttacagtcgttgataaataattattaatttatgtgtaattaatagagttttactgtaataaggaagaagctgtcaggacacttacttgctcttttatgaacactcttttatgaaaattaaaacggagaatactgccagatgagtcatcagtgttcactttctgaactgaatgaaaacctggtgcctttcactgccaggcagatcaagtcctcattaatctggtgtgcagaggatgtcagaaatccaggcgagctgcaaaatgcctgcagttatattatcgatcctgacagtacggcttctgccaaagtgttccatgcagagcgctatggtggcagtggtattcagcgtaatggaggtggtgcacgttgtgggtttgatggtaactaccaggttaaaggaataggaagtaatccgttggttggtgaaggtactgacgaacgtcattctaatggtgcactcggcgctgttcatgcaatatatgaggctttgtggggagaagtactggctcaaatattaccttatagtgctgtgcgggttcgggcggttttacttacagatctctatactgaaaaggcatttgagcgctccggtatgaaatcacgaagagccctgttggtacgtgagcctgttgttcgcccggcgcattttgaacgggcaccatacttccaagtaaaaccggagtattccagtcagttaattcacgatgcctgtcgggttagatctgtgatccacaagctgccaggatatctacctgtaccaccggaagaaattgatgctgaagcacgaactgatccccggatttattgcattgagggattatgtgaactggcacgtcgtgaggcctggcaaatggcattttgtcgaacacgtttcctgagattgacaacttctccttctaatattgcaatggatggcagattaatggattttaacggactcagttgtctgtttccgggagattccccagctgattttgggtataaactaagattagctgaactggcaaaagaaccgatggtacttatgcaagggctgtctgatctctgcttgtatatcggaaaatatatgtttgaccctgacttcactcttgcagcccgtttgaaggttgaggagatatttcagaaaacttttcatgaagcatgttattactgttatctagaactgttgggtattcctggagaatttataacacaaaaagagatacctgatatattgaaacaactggttaacagttttgttgcattactcaataaatactgcgagaaatcacatgcccaagatattgtcaatcaggatggttcaccattgcaaaagttggttgtgacgctaatccatcataggcataatcaaaagcaggcactgaatagtagcatcaagaatgatgtttatttcaccgttgcacaacagtgtttttcccagactatccactggctgacgcaaggcagtaccagacgtcagataaatgcttcattactcctgaaagaaattgaacatcataccatgaaaaggctgcaacccagggaagagctgaggaaagagaatatgtgcgaaaaaattgccatcctgctggataatcatggcgatgatcccctttttttacaagaagcaatttctgatatgaaaaattttatgcttaagttttccagagatgcatttggatatcttgaaccgataagaaacacagtgtaataaatatataagggaaatagtaatcatgtcttatataagggaaaccatcagaggaaaagatgaatggactgtttatgaacagataggttttgcggtcagttgtatgctctacaatcgtaattacagtctgtatccggtgttaaccattcaatactggactgaatatgcgatacagcataatcagattaaattcctgtttgattcacgaggttttccactggcgtatataacctgggcatatcttgaggctgatacggaagcgcgcctgctcagggatccagaattcaggttgcatccgtctgaatggaatgaagatggaaggatctggatcctggatttctgttgtaaaccaggctttggtcgaaaagttattgactatctcatacagcttcagccatggggggaaggagaagtacgatggttaagcaggcgaaagaaaattgtgacatacatccctgagcggctgcataaaacgtagtacctctgaagatacagagataattgtaaattacggggtaaatgcatcgctgatactattttgacaggactctgtattttctggctttgattatagcgtttcggttaatcagtgaaggtattacaggaaaagtgtgagtaagaggagctattttgtttcgtcaggatgctttagaaaacagaaaaatgaagtggcagggacgggcaatattacttcccggaataccactatggttaatcatgctgggaagcattgtgtttattacggcatttctgatgttcattattgttggtacctatagccgccgtgttaatgtcagtggtgaggtcacaacctggccaagagctgtcaatatatattcaggtgtacagggatttgttgtcaggcaatttgttcatgaagggcagttgataaaaaaaggggatcctgtttatctgattgacatcagtaaaagtacacgtagtggtattgtcactgataatcatcggcgggatatagaaaatcagctggttcgtgtggacaacattatttcccgtctggaagaaagtaaaaaaataacgttagataccctggaaaaacaacgtctgcaatacacagatgcgtttcgtcgctcatcagatattatacagcgtgcagaggaagggataaaaataatgaaaaacaatatggagaattacagaaactatcaggcaaaagggctgattaataaagatcagttaactaaccaggtggcattatattatcagcaacaaaacaatcttctcagcctgagcggacagaacgaacagaatgccctgcagataaccactctggagagtcagattcagactcaggctgcagattttgataaccgtatctaccagatggaactgcaacggtacgagttacagaaagaactggttaacactgatgtggagggcgaaattattatccgggcgttgactgacgggaaagttgactccctgagtgtcactgtcgggcaaatggtcaataccggagacagccttctgcaggttattcctgagaacattgaaaactattatcttattctctgggtcccaaatgatgctgttccttatatttcggctggtgacaaagtgaatattcgttatgaagcctttccggcagaaaaatttgggcagttctctgctacggttaaaactatatccaggactcctgcgtcaacacaggaaatgttgacctataagggtgcaccacagaatacgccgggcgcctctgttccctggtataaagtcattgcgatgcctgaaaagcagattatcagatatgacgaaaaatacctccctctggaaaatggaatgaaagccgaaagtacactatttctggaaaaaaggcgtatttaccagtggatgctttctcctttctatgacatgaaacacagtgcaacaggaccgctcaatgactaacgggagtttcagacaaattataaatcagcttgatatgcgctggcgacgtcgtgttccggttattcatcagacggagaccgctgaatgtggactggcctgcctggcaatgatatgcggtcattttggtaagaatattgacctgatatctcttcgccggaagtttaatctctcggcccgtggagcaaaccttgcaggaatcaatggaatagcggagcagctggggatggtcacccgggctctttcactggagctggatgaacttggtgccctcaaaatgccgtgtattctccactgggatttcagtcactttgtcgtgctggtcagcgtaaagcgtaaccgttatgtactgcatgatccggccagaggcagaagatatctcggtcgggaggaaatgagccggtattttacgggcattgcacttgaggtctggcctggaagtgaattcctggcggaaacccagcagatccgcataagtctccgttcactgattaacagtatttacggtattaaaagaacactggcgaaaattttctgtctgtcagttgtaattgaagcaatcaatctggtaatgccggtggggactcagctggttatggatcatgcgattccggcgggggacagagggctgctgacgcttatttctgctggcctgatgttctttatattgctcagggccgcggtgagtatgctgcgtgcatggtcctcactggttatgagcacgctcatcaatatacagtggcagtcgggtctgtttaaccatcttctcagactgccgctggcgttttttgaacgccgtaaattaggtgatatccagtcgcgttttggctcccttgacactttgagggccacctttaccacctgtgtggttggggcaatcatggacagtattatggttgtgggtgtttttgtgatgatgctgttatatggaggatatcttacctggatagtgctcggttttaccatggtttacgttcttattcgtctggtgacatacggctattaccggcaaatatcggaagaaactcttgtcaggggggcccgggccagctcctattttatggaaagcctgtatggtattgccacggtaaaaatccaaggtatggctgggatccggggaacacactggcttaacctgaaaatagatgcgatcaattcaggtattaagttaaccaagatggatttgctcttcggggggataaatacttttgttgccgcctgtgatcaggtggcgattttatggctgggtgcaagccttgtgatcgataatcagatgacaatagggatgtttgtggcatttggttcttttcgtgggcagttttcggatcgggttgcttcgctgaccagttttcttcttcaactgagaataatgagtctgcataatgagcgcattgcagatattgcactacatgaaaaggaagaaaagaaaccggaaattgaaatcgttgctgacatgagcccggtttcactggaaaccactgatttaagctaccggtatgacagccagtcagcacaggtattcagtggtctgaatttgtctgtggctccgggagaaagtgtggctataactggtgcctccggtgccggaaaaaccacattaatgaaagtattatgtggactgtttgaaccagatagtggaaaagtactggttaatggcacggatatacgtcaacttggaataaataattatcaccgtatgatagcctgtgttatgcaggacgaccggctattttcaggatcaattcgtgaaaatatctgtgggtttgcagaagaaacagacgacgaatggatgacagaatgtgccagagcaagtcatattcatgatgtgataatgaaaatgccaatggggtatgaaacgttaataggtgaactgggggaaggtctttccggcggtcaaaaacagcgtatattcattgcccgagctttataccggaaacctggaatattatttatggatgaggctacaagttctcttgatacagaaagtgaacgtttcgtgaatgctgccataaaaaaaatgaatatcacccgggtgattattgcacacagagaaactacgttgagaactgttgacaggattatttctatttaaaatgaggccctttaactgtgtggatgggacgatcgttgacagctagctcagtcctaggtataatgctagcctgaaacggctgccggttaataaggaggtacaacatgcgaaaacgtattctttttattggcccaccgctgtacggtttgttatacccattgatttctctggctcaggcctttcgtgtaatcggacatgatgtagtaattagtagtgctggcaaattcgcgaataaagcagcagaagctggactggttgtttttgatgcagttccaggtttagattcagaggctggatatcgccatcaggaagagttgaggaaaaaaagtaatattattggtcatttctctttttttagcgatgaaatggcagataacctcatcgattttgcaggaaaatggaggccagatttaatagtctatcccccgcttggtccggcaggcccattggttgctgctaaatatagaattccttcagtgatgctggctgttggattcgcgcatacatctgcccatattcagatgttaaaccgttctttaagcaatgcttacaggcggcatggagtcagcggtccactatgtgatttagcatggattgatgttgctcccccaagtatgagcattcttaaaaatgctgaagaaccggttatctcaatgagatatattccttataacggaggtgctgtaaaggaaacatggtgggacagggattctgatcgaaaacgtttactcatcagccttggcactgtaaaaccaatggttgatggtctggagctgatttcatgggttatggattctgcaaatgaagttgatgctgatatcattttgcaacttgcaataaatgctcgtactggattacgaaaactaccatcaaatgtacgtctggttgactggatacctatgggtgtattccttaatggagctgatggatttattcatcatggtggcgcaggtaataccctgacagcgttgtatagtgggataccacagattgtgtttggcgaaggtgcagatcgctctgttaatgcagaaattgttgcgatgcgtgggtgtgggattattccggacaagcatggactgaccagtgatttggtaaatcgcctgctttatgatgattcactacgcttctgttcagatcaggtagccgctgaaatggctgaacaacccagtcctgcagagatcgcagaggttttgatgagaaaattaaaaaacaacgggaaataagaacacgaagacatggccttatctaaaacagaccatacgttatcaataccagaaaaatatattgttatcggtataaataaaaacaacattgataagagatacattctaattttcatttttgtaaaatttcctgtaccacgttgatctacttattcctaaagaaatccattctccatctctaactttcggccttccaccaccagagcttttttttccacgttgacgctgaatttcagaagtatgtgtttgtttaacatactcttcaaagccaagctctgtaaggttcttacttgtccacttagccacacttttagcaattcccatgacttcgttctcgtctaaaggtgcggagaactgcaggttgtaggctttagcgcgttcaatgcaggcttgtagccattggtcatactgcggccagccttggcggatagcgcggtaagcccacttgcgggttttatcgaagagggtgcagttacggcctaaaccgtagtccggcaggatttcgcggtcattggctgcgccaaggtcgaggtaatcggctaaccagtcaagggtatagagctctggctgccagacggtgatctgccagtgcaggtggttcggattcttgcaaattagccctgaataccccgcatctgcgcccaattttttacgcagcgcattctcgatggcggcggcgtatttaaggggggcagctcgaccatccggcgcggtacgtaccgccgtatgcaaggcatacaacaggtgagcatgtccgttctcggggtttttgatggtgagtgtgggcgcaggtgcccccagatcggcccaatcaatcgcggctccggctctgtccacgtcaaagcaaagccagtacatggcgtgaggctgattaaactggatgtattttgcgaggagagcacgctctttaccggcaatgcgaacaccaaactgtaaatcatcggagaagtacggcttgtggggtaaccggtcgttaaaaagcgttaaagcctgattatccaaggctcccagccttatggcggggctgttgttttgcacgctgcatgtgctaatatcctttctaggtttcgacctagccctgaatgtcatgtccgctcgccaaagtagagcatgatttcggggctttgttttttctgccactaagttacacctcaacaacggtttttgtcatccccgacaatccgttattcctgcttgttctcgcacggctttacgctcatactacttcttgtagatacacttgtcactacatcaagaggtgagatgatggccacgattaatattcggatcgatgacgagctgaaaagccgctcttatgccgcactggaaaagctgggcgtaacgccgtccgaggttctgcgccaaacactggaatatgtggcccaaagcggacgtttgccgttccagcaggttttgctgaccgaggatgatgccgatttgatggctatcgttcgggatcgtctggaaaacccacaggcgggcgtaaaggtgtcactggatgagctataaccttgaatttgatccccgagccctgaaggaatggcgcaagctcggggatgatgtccgtctgcagttcaagaaaaaactcgagcaggttctacaacacccgcggatcgataaaaatcgcctgcgagagctgcatgactgctacaaaatcaagctccgtgcatccggttatcgcttggtctatcaggttcgcgatcaaaccattacggtattcgtggtggcggtcggtaagcgcgagcgttctgccgcttacgatgcggccgataaacgcttataaactcatgccgtgaccgcgagaataccgctgttcgtgccgcttggctaattgctcaagcggcgcactgttctgtttaagctctcgacttgctgcgccaagccggtgacttgcatgctcaaggcctgacaaaccctgtcgtttttcgccaactcctgcgaggtaacctcgaacatgcgctgtaagttggcgtagctgtcctgccacgcttgctgctgttgttcgtagtgcctctgtaagctctctaatgcgctcagaagctgctgctccatttcggtcatgaatctcttcaccctgatagataaaaccgcccagaatcgattctgtggcgtctgatgaggttatttggcgctgtacttgatgacctgacgatgttgagcgttcttgtactcgtcgatcttcttcgccccctgcggaaggatcaggtaatacacgctcttgttcttggaatcgtgaattatcgatacgccggctccggtctggctctttaagtcctgcaggatctggctctgctcgctgatttcgttctggcgttcgaccacgatagtcccgagataccaagctactccaatcaatatcgcaaacaggatcccacttaatgccaggctgtacagccatgtcattccgactaagcggtgtatctgttttagctggctgtcgttctcttcttggatagcggtctgtatgttccctgagcttaatttcaaggcctcggtgatacgtttctcgtgcttctcgaatgcgttcgcgacgctcgttgcggtagtcttggcttgctgcttcgatttgctctcgaactcccgcgctaaatttaaaatctcgctcatacagcactccttttaagcgaatattcgggccacctgccggatcagcaatactgatactggatttggtttcccgtacgaccgacaatccggcatcggtaaggtgggaaatcacccctttacgatccgtaatttctccctgctcaatcaagctgattagccctttggtaatggcttccgctgcctgctgtttgttgcgaggaaggtcattagagggggttaatgctcggcgattagcagggtcattcgggtcgcgtaacccaagccggtcattggtgagggtttgccatgcgttaacacgaggccggtcagcccgatcaaagtaaggttgtagccgttttccgctctgcaattcgatgttcgggataacaaaattcaattcaagacgccctttgtcctgatgttgaacccagaggcaggcatactggtctttatctagaccggtcatcaatgtctgctcccattcatccatcaatcgctgcttttcgccttcgggtaaatcactctcctgaaaagagagcacgccagaggtataagttcgggcaaattcgcagccatcaatcagctctttgacgtgctcagggttaccccgtaacaccgtcgcttgttcgcgctgacgatcagggcccagaaggtaatcgacaggaccactcccgccaccggcaccacgaccatgaatcttaacgatcacgatgttgctccagcagttcggcaagatgttggtcaatgctattgagcaccgctaacaacgacacccgttcttgcggcggtaagccatgctgattcaagtaacgggctatttgattgaggttattaccgatcccgctgacctgacgtaacaaggtcgggtctacggtaggtagcggacgacgccgagctgtacgcgattcgcctaagccaacggctcgtaaccactcggccaaatgcttacggtcacagcgttcaagtagccgctgatgctccgcttcggtgagtctga

SEQ ID NO: 16 - protein, 75 aa, Escherichia coli
MREITESQLRYISGAGGAPATSANAAGAAAIVGALAGIPGGPLGVVVGAVSAGLTTAIGSTVGSGSASSSAGGGS

SEQ ID NO: 17 - protein, 77 aa, Escherichia coli
MREISDNMLDSVKGGMNLNGLPASTNVIDLRGKDMGTYIDANGACWAPDTPSIIMYPGGSGPSYSMSSSTSSANSGS